SEQUENCE LISTING (37 sequences)

SEQ ID NO: 1 - protein, 157 aa, Artificial Sequence
  CD8 specific DARPin
DLGKKLLEAARAGQDDEVRILMTNGADVNALDQAGSTPLHLAAWHGHLEIVEVLLKYGADVNASDIIGQTPLHLAALNGHLEIVEVLLKNGADVNARDRLGETPLHLAAFDGHLEIVEVLLKYDADVNAQDKFGKTPFDLAIDNGNEDIAEVLQKAA

SEQ ID NO: 2 - protein, 157 aa, Artificial Sequence
  CD8 specific DARPin
DLGKKLLEAARAGQDDEVRILMTNGADVNAQDLQGNTPLHLAAWHGHLEIVEVLLKYGADVNARDVKGNTPLHLAANVGHLEIVEVLLKYGADVNATDNWGHTPLHLAAFWGHLEIVEVLLKYGADVNAQDKFGKTPFDLAIDNGNEDIAEVLQKAA

SEQ ID NO: 3 - protein, 157 aa, Artificial Sequence
  CD8 specific DARPin
DLGKKLLEAARAGQDDEVRILMTNGADVNAEDTQGNTPLHLAAWHGHLEIVEVLLKYGVDVNASDIIGQTPLHLAALNGHLEIVEVLLKYGADVNAFDRFGDTPLHLAAWTGHLKIVEVLLKHGADVNAQDKFGKTPFDLAIDNGNEDIARSAAESS

SEQ ID NO: 4 - protein, 157 aa, Artificial Sequence
  CD8 specific DARPin
DLGKKLLEAVRAGKDDEVRILMANGADVNAEDTQGNTPLHLVAWHGHLEIVEVLLKYGADVNASDIIGQTPLHLAALNGHLEIVEVLLKYGADVNAWDRHGHTPLHLAAYFGHLEIVEVLLKNGADVNAQDKFGKTPFDLAIDNGNEDIAEVLQKAA

SEQ ID NO: 5 - protein, 157 aa, Artificial Sequence
  CD8 specific DARPin
DLGKKLLEAARAGQDDEVRILMANGADVNAIDRFGTTPLHLAAWHGHLEIVEVLLKNGADVNTQDSQGMTPLHLAANIGHLEIVEVLLKYGADVNALDRWGLTPLHLAAWFGHLEIVEVLLKNGADVNAQDKFGKTPFDLAIDNGNEDIAEVLQKAA

SEQ ID NO: 6 - protein, 157 aa, Artificial Sequence
  CD8 specific DARPin
DLGKKLLEAARAGQNDEVRILMANGVDVNAKDVNGSTPLHLAAWHGHLEIVEVLLKYGADVNASDIIGQTPLHLAALNGHLEIVEVLLKNGADVNALDHYGLTPLHLAAWDGHLEIVEVLLKYGADVNAQDKFGKTPFDLAIDNGNEDIAEVLQKAA

SEQ ID NO: 7 - protein, 157 aa, Artificial Sequence
  CD8 specific DARPin
DLGKKLLEAARAGQDDEVRILMANGADVNAEDTQGNTPLHLAAWHGHLEIVEVLLKYGADVNASDIIGQTPLHLAALNGHLEIVEVLLKYGADVNAWDRHGHTPLHLAAAFGHQEIVEVLLKNGADVNAQDKFGKTPFDLAIDNGNEDIAEVLQKAA

SEQ ID NO: 8 - protein, 157 aa, Artificial Sequence
  CD8 specific DARPin
DLGKKLLEAARAGQDDEVRILMANGTDVNAHDKLGQTPLHLAAWHGHLEIVEVLLKYGADVNASDIIGQTPLHLAAVSGHLEIVEVLLKNGADVNAHDRHGETPLHLAAWDDHLEIVEVLLKYGADVNAQDKFGKTPFDLAIDNGNEDIAEVLPESS

SEQ ID NO: 9 - protein, 157 aa, Artificial Sequence
  CD8 specific DARPin
DLGKKLLEAARAGQDDEVRILMTNGADVNASDADGTTPLHLAAWNGHLEIVEVLLKYGADVNARDVTGNTPLHLAAQVGHLEIVEVLLKYGADVNAHDRWGLTPLHLAAHQGHLEIVEVLLKYGADVNAQDKFGKTPFDLAIDNGNEDIAEVLQKAA

SEQ ID NO: 10 - protein, 157 aa, Artificial Sequence
  CD8 specific DARPin
DLGKKLLEASRAGQDDEVRILMANGADVNANDSFGSTPLHLAAWHGHLEIVEVLLKHGADINAQDTHGHTPLHLAANTGHLEIVEVLLKNGADVNAVDSFGHTPLHLAAFWGHLEIVEVLLKHGADVNAQDKFGKTPFDLAIDNGNEDIAEVLQKAA

SEQ ID NO: 11 - protein, 157 aa, Artificial Sequence
  CD8 specific DARPin
DLGKKLLEAARAGQDDEVRILMANGADVNAIDRFGTTPLHLAAWHGHLEIVEVLLKNGADVNARDVKGNTPLHLTANVGHLEIVEVLLKYGADVNATDNWGHTPLHLATFWGHLEIVEVLLKYGADVNAQDKFGKTPFDLAIDNGNEDIAEVLQKAA

SEQ ID NO: 12 - protein, 157 aa, Artificial Sequence
  CD8 specific DARPin
DLGKKLLEAARAGQDDEVRILMANGADVNAIDRFGTTPLHLAAWHGHLEIVEVLLKYGADVNARDVKGNTPLHLTANVGHLEIVEVLLKYGADVNATDNWGHTPLHLAAFWGHLEIVEVLLKYGADVNAQDKFGKTPFDLAIDNGNEDIAEVLQKAA

SEQ ID NO: 13 - protein, 157 aa, Artificial Sequence
  CD8 specific DARPin
DLGKKLLEAARAGQDDEVRILMANGADVNARDKVGSTPLHLAAWHGHLEIVEVLLKYGADVNASDIIGQTPLHLAATNGHLEIVEVLLKYGADVNARDRHGITPLHLAAWLGHLEIVEVLLKNGADVNAQDKFGKTPFDLAIDNGNEDIAEVLQKAA

SEQ ID NO: 14 - protein, 157 aa, Artificial Sequence
  CD8 specific DARPin
DLGKKLLEAARAGQDDEVRILMANGADVNASDSVGNTPLHLAAWHGHLEIVDVLLKYGADVNASDVSGQTPMHLAALQGHLEIVEVLLKYGADVNTHDRWGLTPLHLAAHQGHLEIVEVLLKHGADVNAQDKFGKTPFDLAIDNGNEDIAEVLQKAA

SEQ ID NO: 15 - protein, 157 aa, Artificial Sequence
  CD8 specific DARPin
DLGKKLLEAARAGQDDEVRILMANGADVNAHDYVGATPLHLAAWHGHLEIVEVLLKYGADVNAQDQAGFTPLHLAAIDGHLEIVEVLLKYGADVNAQDRNGVTPLHLAAWMGHLEIVEVLLKYGADVNAQDKFGKTPFDLAIDNGNEDIAEVLQKAA

SEQ ID NO: 16 - protein, 157 aa, Artificial Sequence
  CD8 specific DARPin
DLGKKLLEAARAGQDDEVRILMANGVDVNAKDVNGSTPLHLAAWHGHLEIVEVLLKHGADVNARDVKGNTPLHLAANVGHLEIVEVLLKYGADVNATDNWGHTPLHLAAFWGHLEIVEVLLKYGADVNAQDKFGKTPFDLAIDNGNEDIAEVLQKAA

SEQ ID NO: 17 - protein, 157 aa, Artificial Sequence
  CD8 specific DARPin
DLGKKLLEAARAGQDDEVRILMANGADVNAVDKVGNTPLHLVAWHGHLEIVEVLLKYSADVNATDTIDKTPLHLAADNGHLEIVEVLLKHGADVNALDRHGFTPLHLAAFMGHLEIVEVLLKYDADVNAQDKFGKTPFDLAIDNGNEDIAEVLQKAA

SEQ ID NO: 18 - protein, 157 aa, Artificial Sequence
  CD8 specific DARPin
DLGKKLLEAARAGQDDEVRILMANGADVNAVDLVGSTPLHLAAWIGHLEIVEVLLKHGVDVNAIDITGSTPLHLAAVIGHLEIVEVLLKYGADVNASDRHGVTPLHLAAFQGHLEIVEVLLKHGADVNAQDKFGKTPFDLAIDNGNEDIAEVLQKAA

SEQ ID NO: 19 - protein, 157 aa, Artificial Sequence
  CD8 specific DARPin
DLGKKLLEAARAGQDDEVRILMANGADVNAEDTQGNTPLHLAAWHGHLEIVEVLLKYGADVNASDIIGQTPLHLAALNGHLEIVEVLLKNGADVNARDRLGETPLHLAVFDGHLEIVEVLLKYGADVNAQDKFGKTPFDLAIDNGNEDIAEVLQKAA

SEQ ID NO: 20 - protein, 157 aa, Artificial Sequence
  CD8 specific DARPin
DLGKKPLEAARAGQDDEVRILMANGADVNAEDTQGNTPLHLAAWHGHLEIVEVLLKYGADVNASDIIGQTPLHLAALNGHLEIVEVLLKHGADVNAHDRHGYTPLHLAAFLGHLEIVEVLLKYGADVNAQDKFGKTPFDLAIDNGNEDIAEVLQKAA

SEQ ID NO: 21 - protein, 157 aa, Artificial Sequence
  CD8 specific DARPin
DLGKKLLEAARAGQDDEVRILMTNGVDVNAQDQNGSTPLHLAAWDGHLEIVEVLLKYGADVNARDLLGQTPLHLAAINGHLEIVEVLLKHGADVNASDRYGLTPLHLATWIGHLEIVEVLLKHGADVNAQDKFGKTPFDLAIDNGNEDIAEVLQKAA

SEQ ID NO: 22 - protein, 157 aa, Artificial Sequence
  CD8 specific DARPin
DLGKKLLEAARAGQDDEVRILMANGADVNAEDTQGNTPLHLAAWHGHLEIVEVLLKYGADVNASDIIGQTPLHLAALNGHLEIVEVLLKNGADVNAEDRWGVTPLHLAAWDGHLEIVEVLLKHGADVNAQDKFGKTPFDLAIDNGNEDIAEVLQKAA

SEQ ID NO: 23 - protein, 157 aa, Artificial Sequence
  CD8 specific DARPin
DLGKKLLEATRAGQDDEVRILMTNGADVNALDQAGSTPLHLAAWSGHLEIVEVLLKYGTDVNARDVKGNTPLHLAANVGHLEIVEVLLKYGADVNATDNWGHTPLHLAAFWDHLEIVEVLLKYGADVNAQDKFGKTPFDLAIDNGNEDIAEVLQKAA

SEQ ID NO: 24 - protein, 157 aa, Artificial Sequence
  CD8 specific DARPin
DLGKKLLEAARAGQDDEVRILMANGADVNASDSVGNTPLHLAAWHGHLEIVEVLLKYSADVNASDVSGQTPLHLAALQGHLEIVEVLLKCGADVNAHDRWGLTPLHLAAHQGHLEIVEVLLKYGADVNAQDKFGKTPFDLAIDNGNEDIAEVLQKAA

SEQ ID NO: 25 - protein, 157 aa, Artificial Sequence
  CD8 specific DARPin
DLGKKLLEASRAGQDDEVRILMANGADVNAQDRYGTTPLHLAAWHGHLEIVEVLLKHGADVNANDVKGNTPLHLAANVGHLEIVEVLLKYGADVNAADNWGHTPLHLAAFWGHLEIVEVLLKYGADVNAQDKFGKTPFDLAIDNGNEDIAEVLQKAA

SEQ ID NO: 26 - protein, 157 aa, Artificial Sequence
  CD8 specific DARPin
DLGKKLLEAARAGQDDEVRILIANGADVNASDSVGNTPLHLAAWHGHLEIVEVLLKYGADVNASDVSGQTPLHLAALQGHLEIVEVLLKYGADVNAHDRWGLTPLHLAAHQGHLEIVEVLLKYGADVNAQDKFGKTPFDLAIDNGNEDIAEVLQKAA

SEQ ID NO: 27 - protein, 157 aa, Artificial Sequence
  CD8 specific DARPin
DLGKKLLEVARAGQDDEVRILMANGADVNARDDAGSTPLHLAAWHGHLEIVEVLLKYGADVNAKDIAGYTPLHLAAVQGHLEIVEVLLKYGADVNAKDRHGVTPLHLAAFQSHLEIVEVLLKHGADVNAQDKFGKTPFDLAIDNGNEDIAEVLQKAA

SEQ ID NO: 28 - protein, 157 aa, Artificial Sequence
  CD8 specific DARPin
DLGKKLLEAARAGQDDEVRILMANGADVNAEDTQGNTPLHLAAWHGHLEIVEVLLKYGADVNASDIIGQTPLHLAALNGHLEIVEVLLKYGADVNAVDRYGDTPLHLAAWDGHLEIVEVLLKHGADVNAQDKFGKTPFDLAIDNGNEDIAEVLQKAA

SEQ ID NO: 29 - protein, 186 aa, Artificial Sequence
  H6-HA-63H6-Cys
MRGSHHHHHHGSYPYDVPDYAAAQPADLGKKLLEAARAGQDDEVRILMANGADVNASDSVGNTPLHLAAWHGHLEIVDVLLKYGADVNASDVSGQTPMHLAALQGHLEIVEVLLKYGADVNTHDRWGLTPLHLAAHQGHLEIVEVLLKHGADVNAQDKFGKTPFDLAIDNGNEDIAEVLQKAAGGC

SEQ ID NO: 30 - protein, 196 aa, Artificial Sequence
  H6-HA-63H6-E10
MRGSHHHHHHGSYPYDVPDYAAAQPADLGKKLLEAARAGQDDEVRILMANGADVNASDSVGNTPLHLAAWHGHLEIVDVLLKYGADVNASDVSGQTPMHLAALQGHLEIVEVLLKYGADVNTHDRWGLTPLHLAAHQGHLEIVEVLLKHGADVNAQDKFGKTPFDLAIDNGNEDIAEVLQKAAGGSEEEEEEEEEE

SEQ ID NO: 31 - protein, 206 aa, Artificial Sequence
  H6-HA-63H6-E20
MRGSHHHHHHGSYPYDVPDYAAAQPADLGKKLLEAARAGQDDEVRILMANGADVNASDSVGNTPLHLAAWHGHLEIVDVLLKYGADVNASDVSGQTPMHLAALQGHLEIVEVLLKYGADVNTHDRWGLTPLHLAAHQGHLEIVEVLLKHGADVNAQDKFGKTPFDLAIDNGNEDIAEVLQKAAGGSEEEEEEEEEEEEEEEEEEEE

SEQ ID NO: 32 - protein, 33 aa, Artificial Sequence
  Consensus sequence
  MISC_FEATURE (2)..(2): any amino acid, preferably an amino acid selected from the group consisting of A, C, D, G, N, P, S, T, and V
  MISC_FEATURE (3)..(3): any amino acid
  MISC_FEATURE (5)..(5): any amino acid
  MISC_FEATURE (6)..(6): any amino acid
  MISC_FEATURE (7)..(7): any amino acid, preferably an amino acid selected from the group consisting of A, C, D, G, N, P, S, T, and V, more preferably G
  MISC_FEATURE (8)..(8): any amino acid
  MISC_FEATURE (11)..(11): any amino acid, preferably an amino acid selected from the group consisting of A, C, F, G, H, I, K, L, M, R, T, V, W, Y, more preferably L
  MISC_FEATURE (14)..(14): any amino acid, preferably an amino acid selected from the group consisting of A, C, D, G, N, P, S, T, and V, more preferably A or V
  MISC_FEATURE (15)..(15): any amino acid, preferably an amino acid selected from the group consisting of A, C, D, G, N, P, S, T, and V, more preferably A
  MISC_FEATURE (16)..(16): any amino acid
  MISC_FEATURE (17)..(17): any amino acid
  MISC_FEATURE (18)..(18): any amino acid, preferably an amino acid selected from the group consisting of A, C, D, G, N, P, S, T, and V, more preferably G
  MISC_FEATURE (20)..(20): any amino acid, preferably L
  MISC_FEATURE (21)..(21): any amino acid, preferably an amino acid selected from the group consisting of D, E, H, K, and R, more preferably E
  MISC_FEATURE (24)..(24): any amino acid, preferably D or E
  MISC_FEATURE (29)..(29): any amino acid
  MISC_FEATURE (30)..(30): any amino acid, preferably an amino acid selected from the group consisting of A, C, D, G, N, P, S, T, and V, more preferably selected from the group consisting of A, G, and S, more preferably G
  MISC_FEATURE (31)..(31): any amino acid, preferably an amino acid selected from the group consisting of A, C, D, G, N, P, S, T, and V, more preferably A
  MISC_FEATURE (33)..(33): any amino acid, preferably an amino acid selected from the group consisting of I, L, and V, more preferably V
NXXDXXXXTPXHLXXXXXHXXIVXVLLKXXXDX

SEQ ID NO: 33 - protein, 33 aa, Artificial Sequence
  Consensus sequence
  MISC_FEATURE (2)..(2): any amino acid, preferably an amino acid selected from the group consisting of A, C, D, G, N, P, S, T, and V
  MISC_FEATURE (3)..(3): any amino acid
  MISC_FEATURE (5)..(5): any amino acid
  MISC_FEATURE (6)..(6): any amino acid
  MISC_FEATURE (8)..(8): any amino acid
  MISC_FEATURE (14)..(14): A or V
  MISC_FEATURE (15)..(15): any amino acid, preferably an amino acid selected from the group consisting of A, C, D, G, N, P, S, T, and V, more preferably A
  MISC_FEATURE (16)..(16): any amino acid
  MISC_FEATURE (17)..(17): any amino acid
  MISC_FEATURE (20)..(20): any amino acid, preferably L
  MISC_FEATURE (21)..(21): any amino acid, preferably an amino acid selected from the group consisting of D, E, H, K, and R, more preferably E
  MISC_FEATURE (24)..(24): any amino acid, preferably D or E
  MISC_FEATURE (29)..(29): any amino acid
NXXDXXGXTPLHLXXXXGHXXIVXVLLKXGADV

SEQ ID NO: 34 - protein, 33 aa, Artificial Sequence
  Consensus sequence
  MISC_FEATURE (2)..(2): any amino acid, preferably an amino acid selected from the group consisting of A, C, D, G, N, P, S, T, and V
  MISC_FEATURE (3)..(3): any amino acid
  MISC_FEATURE (5)..(5): any amino acid
  MISC_FEATURE (6)..(6): any amino acid
  MISC_FEATURE (8)..(8): any amino acid
  MISC_FEATURE (14)..(14): A or V
  MISC_FEATURE (16)..(16): any amino acid
  MISC_FEATURE (17)..(17): any amino acid
  MISC_FEATURE (24)..(24): D or E
  MISC_FEATURE (29)..(29): any amino acid
NXXDXXGXTPLHLXAXXGHLEIVXVLLKXGADV

SEQ ID NO: 35 - protein, 33 aa, Artificial Sequence
  Consensus sequence
  MISC_FEATURE (3)..(3): any amino acid
  MISC_FEATURE (5)..(5): any amino acid
  MISC_FEATURE (6)..(6): any amino acid
  MISC_FEATURE (8)..(8): any amino acid
  MISC_FEATURE (14)..(14): A or V
  MISC_FEATURE (24)..(24): D or E
  MISC_FEATURE (29)..(29): any amino acid, preferably an amino acid selected from the group consisting of Y, H, and N
NAXDXXGXTPLHLXAWHGHLEIVXVLLKXGADV

SEQ ID NO: 36 - protein, 33 aa, Artificial Sequence
  Consensus sequence
  MISC_FEATURE (3)..(3): any amino acid
  MISC_FEATURE (5)..(5): any amino acid
  MISC_FEATURE (6)..(6): any amino acid
  MISC_FEATURE (8)..(8): any amino acid
  MISC_FEATURE (16)..(16): any amino acid
  MISC_FEATURE (17)..(17): any amino acid
  MISC_FEATURE (29)..(29): any amino acid, preferably an amino acid selected from the group consisting of Y, H, and N
NAXDXXGXTPLHLAAXXGHLEIVEVLLKXGADV

SEQ ID NO: 37 - protein, 33 aa, Artificial Sequence
  Consensus sequence
  MISC_FEATURE (2)..(2): any amino acid, preferably an amino acid selected from the group consisting of A, C, D, G, N, P, S, T, and V
  MISC_FEATURE (3)..(3): any amino acid
  MISC_FEATURE (5)..(5): any amino acid
  MISC_FEATURE (6)..(6): any amino acid
  MISC_FEATURE (8)..(8): any amino acid
  MISC_FEATURE (16)..(16): any amino acid
  MISC_FEATURE (17)..(17): any amino acid
  MISC_FEATURE (29)..(29): any amino acid, preferably an amino acid selected from the group consisting of Y, H, and N
NXXDXXGXTPLHLAAXXGHLEIVEVLLKXGADV

(19) United States
(12) Patent Application Publication
(10) Pub. No.: US 2023/0049655 A1
(43) Pub. Date: Feb. 16, 2023

(54) IN VITRO AND IN VIVO GENE DELIVERY TO IMMUNE EFFECTOR CELLS USING NANOPARTICLES FUNCTIONALIZED WITH DESIGNED ANKYRIN REPEAT PROTEINS (DARPINS)

(71) Applicant: BIONTECH CELL & GENE THERAPIES GMBH, Mainz (DE)

(72) Inventors: Annika FRANK, Frankfurt am Main (DE); Jessica HARTMANN, Bad Soden (DE); Christian BUCHHOLZ, Frankfurt am Main (DE); Benjamin RENGSTL, Mainz (DE); Arne Marten BILLMEIER, Mainz (DE); Ugur SAHIN, Mainz (DE); Hans-Ulrich SCHMOLDT, Mainz (DE); Philipp HELLER, Mainz (DE); Bonny Gaby LUI, Mainz (DE); Anne SCHLEGEL, Mainz (DE)

(21) Appl. No.: 17/781,479

(22) PCT Filed: Dec. 22, 2020
(86) PCT No.: PCT/EP2020/087627

§ 371 Date: Jun. 1, 2022

(30) Foreign Application Priority Data
Dec. 27, 2019 (EP) PCT/EP2019/087110

(51) Int. Cl. A61K 35/17; A61P 35/00; C07K 14/47; C07K 14/705; C12N 15/90; C12N 15/88; B82Y 5/00; C12N 5/0783; C12N 9/22

(52) U.S. Cl. CPC A61K 35/17; A61P 35/00; C07K 14/47; C07K 14/70517; C12N 15/907; C12N 15/88; B82Y 5/00; C12N 5/0636; C12N 9/22

(57) ABSTRACT

The present disclosure generally relates to therapies involving immune effector cells such as T cells engineered to express antigen receptors such as T cell receptors (TCRs) or chimeric antigen receptors (CARs). It is demonstrated herein that such antigen receptor-engineered immune effector cells may be generated in vitro/ex vivo as well as in vitro by delivering nucleic acid encoding an antigen receptor for genetic modification to cells using particles comprising the nucleic acid and a targeting molecule for targeting the immune effector cells, wherein the targeting molecule is a designed ankyrin repeat protein (DARPin). In particular, DARPins are described herein which are high-affinity binders for CDS binding to the CDS receptor on human and non-human primate (NHP) cells. Nanoparticles functionalized with CD8− targeting DARPins (CDS-DARPin) can deliver genes exclusively and specifically to human CD8⁺ T cells in vitro and in vivo.

### TECHNICAL FIELD

The present disclosure generally relates to therapies involving immune effector cells such as T cells engineered to express antigen receptors such as T cell receptors (TCRs) or chimeric antigen receptors (CARs). In one embodiment, the immune effector cells are genetically modified to express the antigen receptor. Such genetic modification may be effected ex vivo or in vitro and subsequently the immune effector cells may be administered to a subject in need of treatment, or may be effected in vivo in a subject in need of treatment. These methods are, in particular, useful for the treatment of cancers characterized by diseased cells expressing an antigen the antigen receptor is directed to. It is demonstrated herein that such antigen receptor-engineered immune effector cells may be generated in vitro/ex vivo as well as in vitro by delivering nucleic acid encoding an antigen receptor for genetic modification to cells using particles comprising the nucleic acid and a targeting molecule for targeting the immune effector cells, wherein the targeting molecule is a designed ankyrin repeat protein (DARPin). In particular, DARPins are described herein which are high-affinity binders for CD8 binding to the CD8 receptor on human and non-human primate (NHP) cells. Nanoparticles functionalized with CD8-targeting DARPins (CD8-DARPin) can deliver genes exclusively and specifically to human CD8⁺ T cells in vitro and in vivo. The antigen receptor-engineered immune effector cells may be provided to a subject by administering the antigen receptor-engineered immune effector cells or by generating the antigen receptor-engineered immune effector cells in the subject. In one embodiment, the antigen receptor-engineered immune effector cells are generated in the subject treated. Furthermore, target antigen for the antigen receptor may be provided to a subject by administering to the subject an antigen targeted by the antigen receptor, a polynucleotide encoding the antigen, or cells expressing the antigen. The antigen to which the antigen receptor is targeted may comprise a naturally occurring antigen or a variant thereof, or a fragment of the naturally occurring antigen or variant thereof. In one particularly preferred embodiment, the polynucleotide encoding the antigen is RNA. The methods and agents described herein are, in particular, useful for the treatment of diseases characterized by diseased cells expressing an antigen the antigen receptor or antigen receptor-engineered immune effector cells are directed to.

### BACKGROUND

The immune system plays an important role in cancer, autoimmunity, allergy as well as in pathogen-associated diseases. T cells and NK cells are important mediators of anti-tumor immune responses. CD8⁺ T cells and NK cells can directly lyse tumor cells. CD4⁺ T cells, on the other hand, can mediate the influx of different immune subsets including CD8⁺ T cells and NK cells into the tumor. CD4⁺ T cells are able to license dendritic cells (DCs) for the priming of anti-tumor CD8⁺ T cell responses and can act directly on tumor cells via IFNγ mediated MHC upregulation and growth inhibition. CD8⁺ as well as CD4⁺ tumor specific T-cell responses can be induced via vaccination or by adoptive transfer of T cells.

Adoptive cell transfer (ACT) based immunotherapy can be broadly defined as a form of passive immunization with previously sensitized T cells that are transferred to non-immune recipients or to the autologous host after ex vivo expansion from low precursor frequencies to clinically relevant cell numbers. Cell types that have been used for ACT experiments are lymphokine-activated killer (LAK) cells (Mule, J. J. et al. (1984) Science 225, 1487-1489; Rosenberg, S. A. et al. (1985) N. Engl. J. Med. 313, 1485-1492), tumor-infiltrating lymphocytes (TILs) (Rosenberg, S. A. et al. (1994) J. Natl. Cancer Inst. 86, 1159-1166), donor lymphocytes after hematopoietic stem cell transplantation (HSCT) as well as tumor-specific T cell lines or clones (Dudley, M. E. et al. (2001) J. Immunother. 24, 363-373; Yee, C. et al. (2002) Proc. Natl. Acad. Sci. U.S.A. 99, 16168-16173). An alternative approach is the adoptive transfer of autologous T cells reprogrammed to express a tumor-reactive immunoreceptor of defined specificity during short-time ex vivo culture followed by reinfusion into the patient (Kershaw M. H. et al. (2013) Nature Reviews Cancer 13 (8):525-41). This strategy makes ACT applicable to a variety of common malignancies even if tumor-reactive T cells are absent in the patient. For example, adoptive transfer of chimeric antigen receptor modified T cells (CAR T cells) is investigated in an extensive number of clinical trials worldwide (FIG. 1, left side). Chimeric antigen receptors (CARs) are a type of antigen-targeted receptor composed of intracellular T cell signaling domains fused to extracellular antigen-binding moieties, most commonly single-chain variable fragments (scFvs) from monoclonal antibodies. CARs directly recognize cell surface antigens, independent of MHC-mediated presentation, permitting the use of a single receptor construct specific for any given antigen in all patients. In general, CARs fuse antigen-recognition domains to the CD3ζ activation chain of the T cell receptor (TCR) complex and comprise secondary costimulatory signals in tandem with CD3ζ, including intracellular domains from CD28 or a variety of TNF receptor family molecules such as 4-1BB (CD137) and OX40 (CD134). CARs dramatically improved antitumor efficacy, showing remarkable clinical efficacy especially in patients suffering from hematological malignancies (Hartmann, J. et al. EMBO Mol. Med. 9, 1183-1197 (2017)). Recently, two CAR T-cell therapies have received approval for the treatment of B-cell acute lymphoblastic leukaemia (Kymriah®) and diffuse large B-cell lymphoma (Yescarta®) by the FDA and EMA (Zheng, P. et al. Drug. Discov. Today 6, 1175-1182 (2018)). For solid tumors adoptive transfer of T cells, however, has shown limited efficacy so far and requires improvement (Newick, K. et al. Annu, Rev. Med. 68, 139-152 (2017)).

Recently, receptor-targeted lentiviral vectors (LVs) were shown to enable selective gene transfer into particular types of lymphocytes in vivo. The LVs are pseudotyped by single chain variable fragments (scFv) directed against receptors on the desired target cell and another component of the envelope protein mediates membrane fusion with the target cell upon binding. This technique reduces the generation of CAR T cells to a single in vivo transduction process. Unfortunately, the production of such LVs is still a laborious and cost-intensive process, although apheresis and ex vivo handling of patient's own T cells can be avoided. Another strategy is to create artificial delivery systems (non-viral vectors) based on lipids or polymers which can mimic LV functions (FIG. 1, right side). Such nanoparticles (NP) need to be functionalized via display/attachment of targeting ligands onto their surface to mediate receptor-specific binding. The targeting ligand could be derived from a parental antibody, e.g. a scFv. In contrast to LVs, binding of the targeting ligand to its receptor needs to induce receptor-mediated endocytosis and trafficking to allow for NP uptake. In fact, receptor-targeted LVs are designed to not mediate endocytosis to avoid endosomal trafficking and lysis. On the other hand, there are NP variants (mostly polymer- or lipid-based), so called polyplexes (PLX, FIG. 2 upper panel) or lipid NPs (LNPs, FIG. 2 lower panel), that bear the potential of escape from endosomes. To mimic retroviral vectors completely and hence to allow for genome engineering via NP-mediated gene delivery, the cargo needs to consist of gene editing tools like CRISPR/Cas9 (or related) or transposon systems like sleeping beauty or piggy bag. Nevertheless, also delivery of mRNA is an option to induce transient expression of therapeutic receptors like CARs or T-cell receptors (TCR). In fact, first studies could recently show that NP are able to generate CAR T cells in vivo. Again the initial efficiency of this process is very low and only CD19 can be targeted so far as circulating B cells display target cells stimulating and expanding the low amount of in vivo generated CAR T cells. Also recently, we developed a CAR vaccine concept (CARVac) that is based on nanoparticle-mediated delivery of mRNA for in vivo display of CAR antigen on professional antigen presenting cells to induce in vivo expansion of CAR T cells (FIG. 3). This technology shall not only enable efficient treatment of non-hematological tumors with CAR T cells but also overcome the hurdle of low efficiencies of in vivo generation of CAR T cells, as CARVac can expand low CAR T-cell numbers up to a therapeutically sufficient level. Moreover, the whole concept can be transferred to other immunoreceptors like TCRs.

Taken together, this approach might facilitate a new way of genetic engineering of patient's own T cells and possibly shifting the whole concept from personalized medicine to an off-the shelf therapy in the future.

Despite of its success, the overall in vivo transduction efficiency of the CD8-specific scFv-LV was rather low. Thus, there is a need for strategies to improve gene delivery to immune effector cells, in particular gene delivery to immune effector cells in vivo. Such gene delivery may be useful in cytotoxic T cell targeting, which will be especially important with regard to the further development of in vivo CAR T cell generation.

We here describe novel high-affinity binders which are designed ankyrin repeat protein (DARPin)-based molecules for targeting surface antigens on immune effector cells. In particular, we describe high-affinity binders for CD8 consisting of DARPins, which were selected to bind to the CD8 receptor of human and non-human primate (NHP) cells. These binders were identified by ribosome display screening of DARPin libraries using recombinant human CD8 followed by receptor binding analysis on primary lymphocytes, Different NPs were then functionalized by different coupling strategies with CD8-targeting DARPins (CD8-DARPin) which delivered genes exclusively and specifically to human CD8⁺ T cells in vitro and in vivo. Functionalizing particles carrying a cargo for genetic modification of immune effector cells with the binders described herein results in the specific delivery of the cargo to and modification of the immune effector cells.

### SUMMARY

The present invention generally embraces the treatment of diseases by targeting cells such as diseased cells expressing an antigen such as a tumor antigen. The target cells may express the antigen on the cell surface for recognition by a chimeric antigen receptor (CAR) or in the context of MHC for recognition by a T cell receptor (TCR). The methods provide for the selective eradication of such cells expressing an antigen, thereby minimizing adverse effects to normal cells not expressing the antigen. Immune effector cells genetically modified to express a chimeric antigen receptor (CAR) or a T cell receptor (TCR) targeting the cells through binding to the antigen (or a procession product thereof) are provided to a subject such as by administration of genetically modified immune effector cells to the subject or generation of genetically modified immune effector cells in the subject. Genetic modification is achieved using particles comprising nucleic acid encoding an antigen receptor for genetic modification and a targeting molecule for targeting the immune effector cells, wherein the targeting molecule is a designed ankyrin repeat protein (DARPin). The particles may deliver the nucleic acid to cells in vitro/ex vivo as well as in viva A vaccine antigen (which may be the disease-associated antigen or a variant thereof (e.g. a peptide or protein comprising an epitope of the disease-associated antigen), nucleic acid coding therefor, or cells expressing the antigen may be administered to provide (optionally following expression of the nucleic acid by appropriate target cells) antigen for immune effector cell stimulation, priming and/or expansion. Immune effector cells stimulated, primed and/or expanded in the patient are able to recognize and eradicate diseased cells expressing an antigen. In one embodiment, the immune effector cells are CD8⁺ T cells. In one embodiment, the targeting molecules described herein bind to the CD8 receptor on CD8⁺ T cells. In one embodiment, the immune effector cells are directed against a tumor or cancer. In one embodiment, the target cell population or target tissue is tumor cells or tumor tissue, in particular of a solid tumor. In one embodiment, the target antigen is a tumor antigen.

The methods and agents described herein are, in particular, useful for the treatment of diseases characterized by diseased cells expressing an antigen the immune effector cells are directed to. In one embodiment, the immune effector cells by means of a chimeric antigen receptor (CAR) have a binding specificity for vaccine antigen and disease-associated antigen when present on antigen presenting cells and diseased cells, respectively. In one embodiment, the immune effector cells by means of a T cell receptor (TCR) having a binding specificity for a procession product of vaccine antigen and disease-associated antigen when presented on antigen presenting cells and diseased cells, respectively. CARs are molecules that combine specificity for a desired antigen (e.g., tumor antigen) which preferably is antibody-based with a T cell receptor-activating intracellular domain to generate a chimeric protein that exhibits a specific cellular immune activity (e.g., a specific anti-tumor cellular immune activity). Preferably, a cell can be genetically modified to stably express an antigen receptor on its surface, conferring novel antigen specificity that may be MHC independent. In one embodiment, immune effector cells either from a subject to be treated or from a different subject are administered to the subject to be treated. The administered immune effector cells may be genetically modified ex vivo prior to administration or genetically modified in vivo in the subject following administration to express an antigen receptor described herein. In one embodiment, the immune effector cells are endogenous in a subject to be treated (thus, are not administered to the subject to be treated) and are genetically modified in vivo in the subject to express an antigen receptor described herein. Accordingly, immune effector cells may be genetically modified, ex vivo or in vivo, to express an antigen receptor. Thus, such genetic modification with antigen receptor may be effected in vitro and subsequently the immune effector cells administered to a subject in need of treatment or may be effected in vivo in a subject in need of treatment. In one aspect, the present invention generally embraces the treatment of diseases by targeting cells expressing an antigen such as diseased cells, in particular cancer cells expressing a tumor antigen. The target cells may express the antigen on the cell surface or may present a procession product of the antigen. In one embodiment, the antigen is a tumor-associated antigen and the disease is cancer. Such treatment provides for the selective eradication of cells that express an antigen, thereby minimizing adverse effects to normal cells not expressing the antigen. In one embodiment, vaccine antigen, polynucleotide coding therefor or cells expressing vaccine antigen are administered to provide (optionally following expression of the polynucleotide by appropriate target cells) antigen for stimulation, priming and/or expansion of immune effector cells genetically modified to express an antigen receptor, wherein the immune effector cells are targeted to the antigen or a procession product thereof and the immune response is an immune response to a target cell population or target tissue expressing the antigen. In one embodiment, the polynucleotide encoding the vaccine antigen is RNA. Immune effector cells such as T cells stimulated, primed and/or expanded in the patient are able to recognize cells expressing an antigen resulting in the eradication of diseased cells. In one embodiment, vaccine antigen-encoding RNA is targeted to secondary lymphoid organs.

In one aspect, the invention relates to a method for preparing immune effector cells genetically modified to express an antigen receptor, comprising contacting the immune effector cells with particles comprising a nucleic acid encoding the antigen receptor and a targeting molecule for targeting the immune effector cells, wherein the targeting molecule is an ankyrin repeat protein.

In one embodiment, contacting the immune effector cells with the particles delivers the nucleic acid to the immune effector cells.

In one embodiment, the immune effector cells to be genetically modified are present in vivo or in vitro. In one embodiment, the immune effector cells to be genetically modified are present in vivo. In one embodiment, the immune effector cells to be genetically modified are present in vivo in a subject and the method comprises administering the particles to the subject.

In a further aspect, the invention relates to a method for treating a subject comprising:

(i) preparing in vitro immune effector cells genetically modified to express an antigen receptor using a method comprising contacting the immune effector cells with particles comprising a nucleic acid encoding the antigen receptor and a targeting molecule for targeting the immune effector cells, wherein the targeting molecule is an ankyrin repeat protein, and
(ii) administering the immune effector cells genetically modified to express an antigen receptor to the subject.

In one embodiment, contacting the immune effector cells with the particles delivers the nucleic acid to the immune effector cells.

In a further aspect, the invention relates to a method for treating a subject comprising:

administering to the subject particles comprising a nucleic acid encoding an antigen receptor and a targeting molecule for targeting immune effector cells, wherein the targeting molecule is an ankyrin repeat protein.

In one embodiment, the particles deliver the nucleic acid to immune effector cells in the subject.

In one embodiment, delivering the nucleic acid to immune effector cells generates immune effector cells genetically modified to express an antigen receptor in the subject.

In one embodiment, the method described herein is a method of inducing an immune response in the subject. In one embodiment, the immune response is a T cell-mediated immune response. In one embodiment, the immune response is an immune response to a target cell population or target tissue expressing an antigen. In one embodiment, the target cell population or target tissue is cancer cells or cancer tissue. In one embodiment, the cancer cells or cancer tissue is solid cancer.

In a further aspect, the invention relates to a method for treating a subject having a disease, disorder or condition associated with expression or elevated expression of an antigen comprising:

(i) preparing in vitro immune effector cells genetically modified to express an antigen receptor targeting the antigen associated with the disease, disorder or condition or cells expressing the antigen associated with the disease, disorder or condition using a method comprising contacting the immune effector cells with particles comprising a nucleic acid encoding the antigen receptor and a targeting molecule for targeting the immune effector cells, wherein the targeting molecule is an ankyrin repeat protein, and
(ii) administering the immune effector cells genetically modified to express an antigen receptor to the subject.

In one embodiment, contacting the immune effector cells with the particles delivers the nucleic acid to the immune effector cells.

In a further aspect, the invention relates to a method for treating a subject having a disease, disorder or condition associated with expression or elevated expression of an antigen comprising:

administering to the subject particles comprising a nucleic acid encoding an antigen receptor targeting the antigen associated with the disease, disorder or condition or cells expressing the antigen associated with the disease, disorder or condition and a targeting molecule for targeting immune effector cells, wherein the targeting molecule is an ankyrin repeat protein.

In one embodiment, the particles deliver the nucleic acid to immune effector cells in the subject.

In one embodiment, delivering the nucleic acid to immune effector cells generates immune effector cells genetically modified to express an antigen receptor in the subject.

In one embodiment, the disease, disorder or condition is cancer and the antigen associated with the disease, disorder or condition is a tumor antigen. In one embodiment, the disease, disorder or condition is solid cancer.

In one embodiment, the method described herein is a method for treating or preventing cancer in a subject. In one embodiment, the cancer is solid cancer. In one embodiment, the cancer is associated with expression or elevated expression of a tumor antigen targeted by the antigen receptor.

In one embodiment, the method described herein further comprises administering to the subject an antigen targeted by the antigen receptor, a polynucleotide encoding the antigen, or a host cell genetically modified to express the antigen. In one embodiment, the polynucleotide is RNA. In one embodiment, the host cell comprises a polynucleotide encoding the antigen.

In one embodiment of all aspects described herein, the antigen receptor is a chimeric antigen receptor (CAR) or T cell receptor (TCR).

In one embodiment of all aspects described herein, the nucleic acid is RNA.

In one embodiment of all aspects described herein, the nucleic acid is DNA.

In one embodiment of all aspects described herein, the genetic modification is transient or stable.

In one embodiment of all aspects described herein, the genetic modification takes place by a virus-based method, transposon-based method, or a gene editing-based method. In one embodiment, the gene editing-based method involves CRISPR-based gene editing.

In one embodiment of all aspects described herein, the particles are non-viral particles. In one embodiment of all aspects described herein, the particles are lipid-based and/or polymer-based particles. In one embodiment of all aspects described herein, the particles are nanoparticles.

In one embodiment of all aspects described herein, the particles are functionalized with the targeting molecule on their surface. In one embodiment of all aspects described herein, the particles are functionalized with the targeting molecule by linking the targeting molecule to at least one particle-forming component.

In one embodiment of all aspects described herein, the immune effector cells are T cells. In one embodiment of all aspects described herein, the immune effector cells are CD8+ T cells.

In one embodiment of all aspects described herein, the targeting molecule targets CD8.

In one embodiment of all aspects described herein, the targeting molecule comprises a repeat module comprising the repeat consensus sequence:

N X₁ X₂ D X₃ X₄ X₅ X₆ T P X₇ H L X₈ X₉ X₁₀ X₁₁ X₁₂ H X₁₃ X₁₄ I V X₁₅ V L L K X₁₆ X₁₇ X₁₈ D X₁₉,
wherein
X₁ is any amino acid, preferably an amino acid selected from the group consisting of A, C, D, G, N, P, S, T, and V,
X₂ is any amino acid,
X₃ is any amino acid,
X₄ is any amino acid,
X₅ is any amino acid, preferably an amino acid selected from the group consisting of A, C, D, G, N, P, S, T, and V, more preferably G,
X₆ is any amino acid,
X₇ is any amino acid, preferably an amino acid selected from the group consisting of A, C, F, G, H, I, K, L, M, R, T, V, W, Y, more preferably L,
X₈ is any amino acid, preferably an amino acid selected from the group consisting of A, C, D, G, N, P, S, T, and V, more preferably A or V,
X₉ is any amino acid, preferably an amino acid selected from the group consisting of A, C, D, G, N, P, S, T, and V, more preferably A,
X₁₀ is any amino acid,
X₁₁ is any amino acid,
X₁₂ is any amino acid, preferably an amino acid selected from the group consisting of A, C, D, G, N, P, S, T, and V, more preferably G,
X₁₃ is any amino acid, preferably L,
X₁₄ is any amino acid, preferably an amino acid selected from the group consisting of D, E, H, K, and R, more preferably E,
X₁₅ is any amino acid, preferably D or E,
X₁₆ is any amino acid,
X₁₇ is any amino acid, preferably an amino acid selected from the group consisting of A, C, D, G, N, P, S, T, and V, more preferably selected from the group consisting of A, G, and S, more preferably G,
X₁₈ is any amino acid, preferably an amino acid selected from the group consisting of A, C, D, G, N, P, S, T, and V, more preferably A,
X₁₉ is any amino acid, preferably an amino acid selected from the group consisting of I, L, and V, more preferably V.

In one embodiment of all aspects described herein, the targeting molecule comprises a repeat module comprising the repeat consensus sequence:

N X₁ X₂ D X₃ X₄ G X₆ T P L H L X₈ X₉ X₁₀ X₁₁ G H X₁₃ X₁₄ I V X₁₅ V L L K X₁₆ G A D V,
wherein
X₁ is any amino acid, preferably an amino acid selected from the group consisting of A, C, D, G, N, P, S, T, and V,
X₂ is any amino acid,
X₃ is any amino acid,
X₄ is any amino acid,
X₆ is any amino acid,

### X₈ is A or V,

X₉ is any amino acid, preferably an amino acid selected from the group consisting of A, C, D, G, N, P, S, T, and V, more preferably A,
X₁₀ is any amino acid,
X₁₁ is any amino acid,
X₁₃ is any amino acid, preferably L,
X₁₄ is any amino acid, preferably an amino acid selected from the group consisting of D, E, H, K, and R, more preferably E,
X₁₅ is any amino acid, preferably D or E,
X₁₆ is any amino acid.

In one embodiment of all aspects described herein, the targeting molecule comprises a repeat module comprising the repeat consensus sequence:

N X₁ X₂ D X₃ X₄ G X₆ T P L H L X₈ A X₁₀ X₁₁ G H L E I V X₁₅ V L L K X₁₆ G A D V,
wherein
X₁ is any amino acid, preferably an amino acid selected from the group consisting of A, C, D, G, N, P, S, T, and V,
X₂ is any amino acid,
X₃ is any amino acid,
X₄ is any amino acid,
X₆ is any amino acid,

### X₈ is A or V,

X₁₀ is any amino acid,
X₁₁ is any amino acid,

### X₁₅ is D or E,

X₁₆ is any amino acid.

In one embodiment of all aspects described herein, the targeting molecule comprises at least 2 repeat modules each comprising the repeat consensus sequence, which may be identical or different.

In one embodiment of all aspects described herein, the targeting molecule comprises between 2 and 20 repeat modules each comprising the repeat consensus sequence, which may be identical or different.

In one embodiment of all aspects described herein, the targeting molecule comprises 3 repeat modules each comprising the repeat consensus sequence, which may be identical or different.

In one embodiment of all aspects described herein, the targeting molecule comprises 3 repeat modules,

wherein
the first repeat module of the targeting molecule comprises the consensus sequence
N A X₂ D X₃ X₄ G X₆ T P L H L X₈ A W H G H L E I V X₁₅ V L L K X₁₆ G A D V,
the second repeat module of the targeting molecule comprise the consensus sequence
N A X₂ D X₃ X₄ G X₆ T P L H L A A X₁₀ X₁₁ G H L E I V E V L L K X₁₆ G A D V, and
the third repeat module of the targeting molecule comprise the consensus sequence
N X₁ X₂ D X₃ X₄ G X₆ T P L H L A A X₁₀ X₁₁ G H L E I V E V L L K X₁₆ G A D V,
wherein
X₁ is any amino acid, preferably an amino acid selected from the group consisting of A, C, D, G, N, P, S, T, and V,
X₂ is any amino acid,
X₃ is any amino acid,
X₄ is any amino acid,
X₆ is any amino acid,

### X₈ is A or V,

X₁₀ is any amino acid,
X₁₁ is any amino acid,

### X₁₅ is D or E,

X₁₆ is any amino acid, preferably an amino acid selected from the group consisting of Y, H, and N.

In one embodiment of all aspects described herein, the targeting molecule comprises at least one repeat module each comprising a sequence selected from the group of repeat modules 1, 2 and 3 of SEQ ID Nos: 1 to 28 as shown in FIG. 5.

In one embodiment of all aspects described herein, the targeting molecule comprises 3 repeat modules, wherein repeat module 1 is selected from the group of repeat modules 1 of SEQ ID Nos: 1 to 28 as shown in FIG. 5, repeat module 2 is selected from the group of repeat modules 2 of SEQ ID Nos: 1 to 28 as shown in FIG. 5, and repeat module 3 is selected from the group of repeat modules 3 of SEQ ID Nos: 1 to 28 as shown in FIG. 5.

In one embodiment of all aspects described herein, the targeting molecule comprises 3 repeat modules, wherein repeat module 1, repeat module 2, and repeat module 3 are the repeat module 1, repeat module 2, and repeat module 3 of a sequence selected from the group consisting of SEQ ID Nos: 1 to 28 as shown in FIG. 5.

In one embodiment of all aspects described herein, the repeat modules are present in a repeat domain.

In one embodiment of all aspects described herein, the repeat domain further comprises an N- and/or a C-terminal capping module.

In one embodiment of all aspects described herein, the targeting molecule comprises a sequence selected from the group consisting of SEQ ID Nos: 1 to 28, or positions 29 to 127 thereof.

In a further aspect, the invention relates to a molecule comprising an ankyrin repeat protein targeting immune effector cells.

In one embodiment, the immune effector cells are T cells. In one embodiment, the immune effector cells are CD8+ T cells.

In one embodiment, the molecule targets CD8.

In one embodiment, the ankyrin repeat protein comprises a repeat module comprising the repeat consensus sequence:

N X₁ X₂ D X₃ X₄ X₅ X₆ T P X₇ H X₈ X₉ X₁₀ X₁₁ X₁₂ H X₁₃ X₁₄ I V X₁₅ V L L K X₁₆ X₁₇ X₁₈ D X₁₉, wherein
X₁ is any amino acid, preferably an amino acid selected from the group consisting of A, C, D, G, N, P, S, T, and V,
X₂ is any amino acid,
X₃ is any amino acid,
X₄ is any amino acid,
X₅ is any amino acid, preferably an amino acid selected from the group consisting of A, C, D, G, N, P, S, T, and V, more preferably G,
X₆ is any amino acid,
X₇ is any amino acid, preferably an amino acid selected from the group consisting of A, C, F, G, H, I, K, L, M, R, T, V, W, Y, more preferably L,
X₈ is any amino acid, preferably an amino acid selected from the group consisting of A, C, D, G, N, P, S, T, and V, more preferably A or V,
X₉ is any amino acid, preferably an amino acid selected from the group consisting of A, C, D, G, N, P, S, T, and V, more preferably A,
X₁₀ is any amino acid,
X₁₁ is any amino acid,
X₁₂ is any amino acid, preferably an amino acid selected from the group consisting of A, C, D, G, N, P, S, T, and V, more preferably G,
X₁₃ is any amino acid, preferably L,
X₁₄ is any amino acid, preferably an amino acid selected from the group consisting of D, E, H, K, and R, more preferably E,
X₁₅ is any amino acid, preferably D or E,
X₁₆ is any amino acid,
X₁₇ is any amino acid, preferably an amino acid selected from the group consisting of A, C, D, G, N, P, S, T, and V, more preferably selected from the group consisting of A, G, and S, more preferably G,
X₁₈ is any amino acid, preferably an amino acid selected from the group consisting of A, C, D, G, N, P, S, T, and V, more preferably A,
X₁₉ is any amino acid, preferably an amino acid selected from the group consisting of I, L, and V, more preferably V.

In one embodiment, the ankyrin repeat protein comprises a repeat module comprising the repeat consensus sequence:

N X₁ X₂ D X₃ X₄ G X₆ T P L H L X₈ X₉ X₁₀ X₁₁ G H X₁₃ X₁₄ I V X₁₅ V L L K X₁₆ G A D V,
wherein
X₁ is any amino acid, preferably an amino acid selected from the group consisting of A, C, D, G, N, P, S, T, and V,
X₂ is any amino acid,
X₃ is any amino acid,
X₄ is any amino acid,
X₆ is any amino acid,

### X₈ is A or V,

X₉ is any amino acid, preferably an amino acid selected from the group consisting of A, C, D, G, N, P, S, T, and V, more preferably A,
X₁₀ is any amino acid,
X₁₁ is any amino acid,
X₁₃ is any amino acid, preferably L,
X₁₄ is any amino acid, preferably an amino acid selected from the group consisting of D, E, H, K, and R, more preferably E,
X₁₅ is any amino acid, preferably D or E,
X₁₆ is any amino acid.

In one embodiment, the ankyrin repeat protein comprises a repeat module comprising the repeat consensus sequence:

N X₁ X₂ D X₃ X₄ G X₆ T P L H L X₈ A X₁₀ X₁₁ G H L E I V X₁₅ V L L K X₁₆ G A D V,
wherein
X₁ is any amino acid, preferably an amino acid selected from the group consisting of A, C, D, G, N, P, S, T, and V,
X₂ is any amino acid,
X₃ is any amino acid,
X₄ is any amino acid,
X₆ is any amino acid,

### X₈ is A or V,

X₁₀ is any amino acid,
X₁₁ is any amino acid,

### X₁₅ is D or E,

X₁₆ is any amino acid.

In one embodiment, the ankyrin repeat protein comprises at least 2 repeat modules each comprising the repeat consensus sequence, which may be identical or different.

In one embodiment, the ankyrin repeat protein comprises between 2 and 20 repeat modules each comprising the repeat consensus sequence, which may be identical or different.

In one embodiment, the ankyrin repeat protein comprises 3 repeat modules each comprising the repeat consensus sequence, which may be identical or different.

In one embodiment, the ankyrin repeat protein comprises 3 repeat modules, wherein

the first repeat module of the targeting molecule comprises the consensus sequence
N A X₂ D X₃ X₄ G X₆ T P L H L X₈ A W H G H L E I V X₁₅ V L L K X₁₆ G A D V,
the second repeat module of the targeting molecule comprise the consensus sequence
N A X₂ D X₃ X₄ G X₆ T P L H L A A X₁₀ X₁₁ G H L E I V E V L L K X₁₆ G A D V, and
the third repeat module of the targeting molecule comprise the consensus sequence
N X₁ X₂ D X₃ X₄ G X₆ T P L H L A A X₁₀ X₁₁ G H L E I V E V L L K X₁₆ G A D V,
wherein
X₁ is any amino acid, preferably an amino acid selected from the group consisting of A, C, D, G, N, P, S, T, and V,
X₂ is any amino acid,
X₃ is any amino acid,
X₄ is any amino acid,
X₆ is any amino acid,

### X₈ is A or V,

X₁₀ is any amino acid,
X₁₁ is any amino acid,

### X₁₅ is D or E,

X₁₆ is any amino acid, preferably an amino acid selected from the group consisting of Y, H, and N.

In one embodiment, the ankyrin repeat protein comprises at least one repeat module each comprising a sequence selected from the group of repeat modules 1, 2 and 3 of SEQ ID Nos: 1 to 28 as shown in FIG. 5.

In one embodiment, the ankyrin repeat protein comprises 3 repeat modules, wherein repeat module 1 is selected from the group of repeat modules 1 of SEQ ID Nos: 1 to 28 as shown in FIG. 5, repeat module 2 is selected from the group of repeat modules 2 of SEQ ID Nos: 1 to 28 as shown in FIG. 5, and repeat module 3 is selected from the group of repeat modules 3 of SEQ ID Nos: 1 to 28 as shown in FIG. 5.

In one embodiment, the ankyrin repeat protein comprises 3 repeat modules, wherein repeat module 1, repeat module 2, and repeat module 3 are the repeat module 1, repeat module 2, and repeat module 3 of a sequence selected from the group consisting of SEQ ID Nos: 1 to 28 as shown in FIG. 5.

In one embodiment, the repeat modules are present in a repeat domain.

In one embodiment, the repeat domain further comprises an N- and/or a C-terminal capping module.

In one embodiment, the ankyrin repeat protein comprises a sequence selected from the group consisting of SEQ ID Nos: 1 to 28, or positions 29 to 127 thereof.

In one embodiment, the molecule further comprises another peptide or protein component, optionally in fusion with the ankyrin repeat protein.

In one embodiment, the molecule is a polypeptide compound.

In one embodiment, the molecule further comprises a lipid or lipid-like component or another non-peptide component.

In a further aspect, the invention relates to a nucleic acid encoding the molecule described herein.

In a further aspect, the invention relates to a host cell comprising the nucleic acid described herein, which optionally expresses the molecule.

In a further aspect, the invention relates to a particle comprising the molecule described herein.

In one embodiment, the particle further comprises a nucleic acid encoding an antigen receptor. In one embodiment, the antigen receptor is a chimeric antigen receptor (CAR) or T cell receptor (TCR). In one embodiment, the antigen is associated with a disease, disorder or condition. In one embodiment, the antigen is a tumor-associated antigen.

In one embodiment, the nucleic acid is RNA. In one embodiment, the nucleic acid is DNA.

In one embodiment, the particle is a non-viral particle. In one embodiment, the particle is a lipid-based and/or polymer-based particle. In one embodiment, the particle is a nanoparticle.

In one embodiment, the particle is functionalized with the ankyrin repeat protein on its surface. In one embodiment, the particle is functionalized with the ankyrin repeat protein by linking the ankyrin repeat protein to at least one particle-forming component.

In a further aspect, the invention relates to a composition comprising the molecule described herein, the particle described herein, or a plurality thereof.

In a further aspect, the invention relates to a pharmaceutical composition comprising the molecule described herein, the particle described herein, or a plurality thereof.

In a further aspect, the invention relates to a kit comprising the molecule described herein, the nucleic acid described herein, the host cell described herein, the particle described herein, the composition described herein, or the pharmaceutical composition described herein.

In one embodiment, the kit further comprises instructions for using the kit in the method described herein.

In a further aspect, the invention relates to the particle described herein, or a plurality thereof for use in the method described herein.

In a further aspect, the invention relates to the agents and compositions described herein, e.g., targeting molecules, particles, nucleic acid encoding an antigen receptor, and/or antigen, polynucleotide encoding an antigen, or host cell genetically modified to express an antigen, for therapeutic use, in particular for use in the methods described herein.

Other features and advantages of the instant invention will be apparent from the following detailed description and claims.

### BRIEF DESCRIPTION OF THE DRAWINGS

FIG. 1: Comparison of classical and in vivo CAR T-cell therapy FIG. 2: Gene delivery devices: PLXs and LNPs

FIG. 3: Vaccine concept (CARVac)

FIG. 4: CD8-specific binding of DARPins

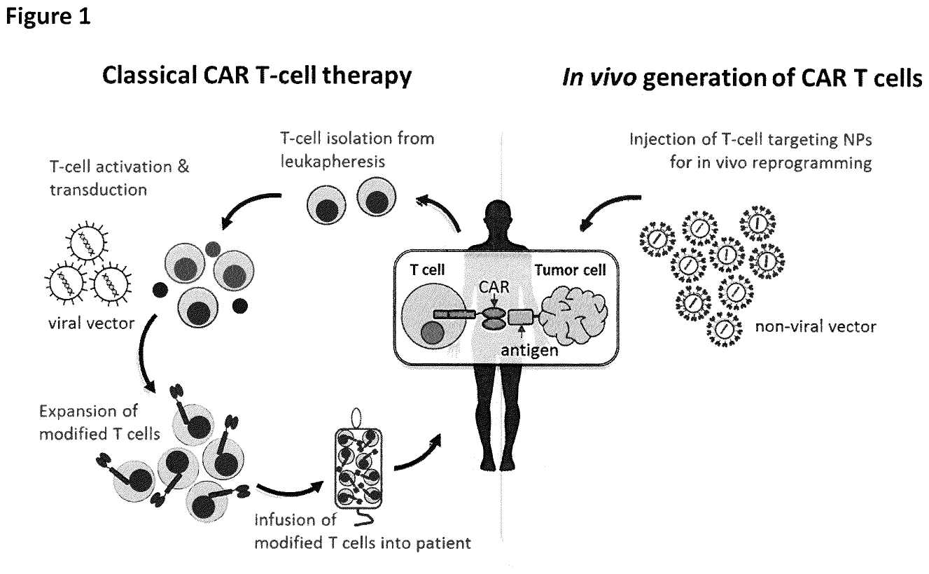

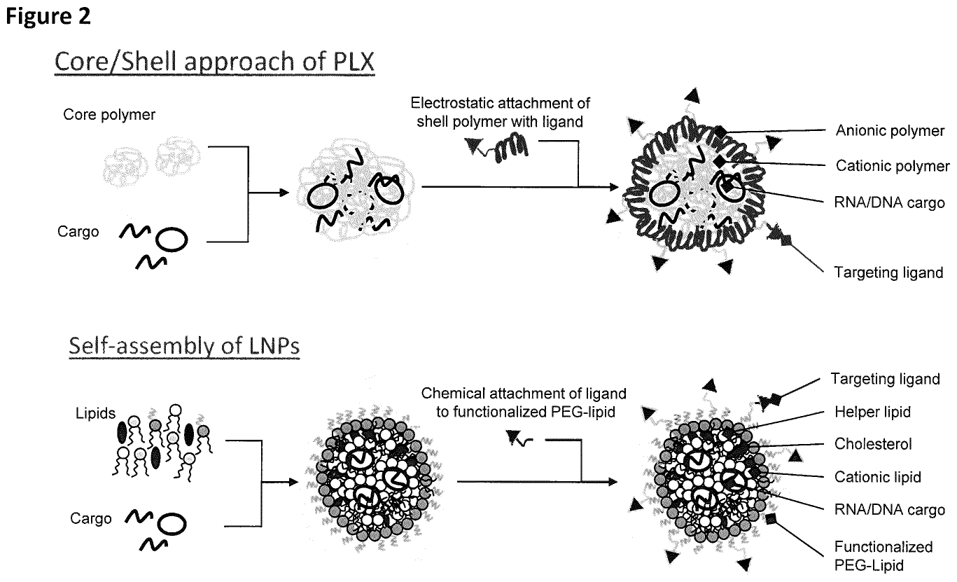

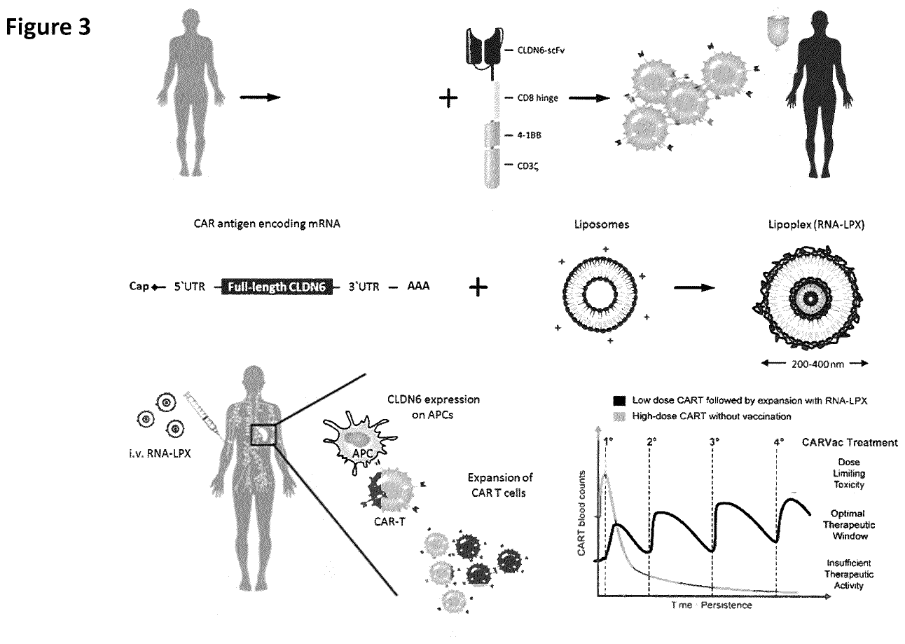

(A) To identify DARPins specifically binding to CD8, crude E. coli lysates of 94 DARPin clones were analyzed for binding to Molt4,8 cells expressing CD8αα and to J67S8ab cells expressing CD8αβ. (B, C) 31 CD8-darpin clones binding equally the CD8 homo- and heterodimer were then tested in a binding assay on primary human (B) and NHP (C) PBMC via flow cytomtery. Bar diagrams show the binding to CD8⁺ and CD8⁻ PBMC for each DARPin. Dotted lines indicate a 2-fold change over background which was used as a threshold to classify DARPins as specific binders when observed on CD8⁺ but not CD8⁻ cells. Arrows indicate DARPins chosen for further analysis. (*) DARPin 5SE11 was analyzed in a separate binding assay using PBMC from another NHP donor.

FIG. 5: Alignment of CD8-specific DARPin Sequences

An Alignment of 28 DARPin sequences is shown.

FIG. 6: Analytical SDS-PAGE of H6-HA-63H6-Cys, H6-HA-63H6-E10 and H6-HA-63H6-E20 DARPins after production and IMAC purification.

A total of 5 μg protein was applied to SDS-PAGE under reducing (+(3-mercaptoethanol) and non-reducing (-β-mercaptoethanol) conditions.

FIG. 7 Specific binding of H6-HA-63H6-Cys, H6-HA-63H6-E10 and H6-HA-63H6-E20 to human CD8+ T-cells.

For flow cytometry analysis, human PBMC of three different donors were stained with anti-CD3-FITC antibody (clone SK-7, BD Bioscience) and anti-CD4-BV421 antibody (clone Okt04, BioLegend). Binding of DARPins to CD8 was detected with an anti-his-APC antibody. Mean fluorescence intensity (MFI) of APC signal on CD8+ T-cells was calculated for data evaluation.

FIG. 8: CD8-specific transfection by LNPs functionalized with DARPins

Attachment of CD8-DARPins to LNPs requires a covalent bond to the PEG-lipid. To allow for click-chemistry reaction, a terminal Cystein (as counterpart to Maleimide as terminal group on the PEG-Lipid, LNP-Mal) was introduced to two selected CD8-DARPin clones (63H6 and 63A4). The constructs were then produced in E. coli and purified. (A) Native PAGE of free DARPin, LNP-Mal alone as well as DARPin plus LNP-Mal and LNP with terminal Azide (LNP-N3, negative control) were applied. (B) DLS data of LNPs with and without attached CD8-DARPin (diameter in grey bars, PDI marked with crosses). (C, D) CD8-DARPin decorated LNPs encapsulating Luciferase-mRNA were tested for transfection efficiency in CD8⁺ and CD8⁻ Jurkat cell lines (C) as well as in human Pan T cells (E) and assessed for Luciferase expression 16 h after administration of 300 ng LNP-formulated RNA per 1×10⁶ cells. LNPs with irrelevant terminal group (N3) or without DARPin attachment served as control.

FIG. 9: CD8-specific transfection by PLX functionalized with DARPins

Attachment of CD8-DARPins to PLX requires electrostatic attraction between the cationic PLX core and an anionic component linked to the targeting ligand. As alternative to coupling to synthetic poly-glutamic-acid (PGA) via reactive ester chemistry as described previously (Smith et al., 2017), we produced CD8-specific DARPin clone 63H6 with an E20 tag recombinantly. (A) Agarose gel electrophoresis showing bands of free DARPin-E20, PLX core alone as well as DARPin plus PLX core at different w/w ratios. (B) DLS data of core PLXs and DARPin-decorated PLXs (diameter in grey bars, PDI marked with crosses). (C) Zeta potential of core PLXs and DARPin-decorated PLXs. (D, E) Transfection potential of CD8-DARPin-decorated PLXs encapsulating Luciferase- and Thy1.1-mRNA (50/50) was tested on CD8⁻ and CD8⁺ Jurkat cell lines. (F, G) CD8-DARPin-decorated PLXs encapsulating Luciferase- and Thy1.1-mRNA (50/50) were tested on human pan T cells with additional viability check and flow cytometric analysis including parallel assessment of CD4⁺ and CD8⁺ T cells. Assays were performed 16 h after administration of 330 ng (Jurkat cells) or 50 ng (primary T cells) PLX-formulated RNA per 1×10⁶ cells.

FIG. 10: CD8-specific transfection by DARPin-decorated LNPs in vivo Immunodeficient mice were transplanted with human PBMC and after 21 days treated with 20 μg mRNA (Luciferase and Thy1.1, 50/50) encapsulated either in non-functionalized or CD8-DARPin-modified LNPs. LNPs were functionalized by Cystein/Maleimide reaction. One day after LNP administration, Luciferase signal was detected via bioluminescence imaging in situ (A) and Thy1.1 expression was assessed by flow cytometric analysis of peripheral blood (B).

FIG. 11: Functionalized nanoparticles as vehicles for delivery of mixed RNA/DNA cargo. CD8⁺ T cells were isolated from peripheral blood of a healthy donor and treated with 50 ng of mixed cargo (minicircle DNA encoding improved YFP and Thy1.1-mRNA, 50/50) encapsulated either in non-functionalized or CD8-DARPinE20-decorated PLX per 1×10⁶ target cells. One day after PLX administration genes of interest were assessed by flow cytometry and cells were activated with CD3/CD28-beads to achieve proliferation. On day 5 post treatment, cell were assessed again via flow cytometry.

### DETAILED DESCRIPTION

Although the present disclosure is described in detail below, it is to be understood that this disclosure is not limited to the particular methodologies, protocols and reagents described herein as these may vary. It is also to be understood that the terminology used herein is for the purpose of describing particular embodiments only, and is not intended to limit the scope of the present disclosure which will be limited only by the appended claims. Unless defined otherwise, all technical and scientific terms used herein have the same meanings as commonly understood by one of ordinary skill in the art.

Preferably, the terms used herein are defined as described in “A multilingual glossary of biotechnological terms: (IUPAC Recommendations)”, H. G. W. Leuenberger, B. Nagel, and H. Kölbl, Eds., Helvetica Chimica Acta, CH-4010 Basel, Switzerland, (1995).

The practice of the present disclosure will employ, unless otherwise indicated, conventional methods of chemistry, biochemistry, cell biology, immunology, and recombinant DNA techniques which are explained in the literature in the field (cf., e.g., Molecular Cloning: A Laboratory Manual, 2nd Edition, J. Sambrook et al. eds., Cold Spring Harbor Laboratory Press, Cold Spring Harbor 1989).

In the following, the elements of the present disclosure will be described. These elements are listed with specific embodiments, however, it should be understood that they may be combined in any manner and in any number to create additional embodiments. The variously described examples and embodiments should not be construed to limit the present disclosure to only the explicitly described embodiments. This description should be understood to disclose and encompass embodiments which combine the explicitly described embodiments with any number of the disclosed elements. Furthermore, any permutations and combinations of all described elements should be considered disclosed by this description unless the context indicates otherwise.

The term “about” means approximately or nearly, and in the context of a numerical value or range set forth herein in one embodiment means ±20%, ±10%, ±5%, or ±3% of the numerical value or range recited or claimed.

The terms “a” and “an” and “the” and similar reference used in the context of describing the disclosure (especially in the context of the claims) are to be construed to cover both the singular and the plural, unless otherwise indicated herein or clearly contradicted by context. Recitation of ranges of values herein is merely intended to serve as a shorthand method of referring individually to each separate value falling within the range. Unless otherwise indicated herein, each individual value is incorporated into the specification as if it was individually recited herein. All methods described herein can be performed in any suitable order unless otherwise indicated herein or otherwise clearly contradicted by context. The use of any and all examples, or exemplary language (e.g., “such as”), provided herein is intended merely to better illustrate the disclosure and does not pose a limitation on the scope of the claims. No language in the specification should be construed as indicating any non-claimed element essential to the practice of the disclosure.

Unless expressly specified otherwise, the term “comprising” is used in the context of the present document to indicate that further members may optionally be present in addition to the members of the list introduced by “comprising”. It is, however, contemplated as a specific embodiment of the present disclosure that the term “comprising” encompasses the possibility of no further members being present, i.e., for the purpose of this embodiment “comprising” is to be understood as having the meaning of “consisting of”.

Several documents are cited throughout the text of this specification. Each of the documents cited herein (including all patents, patent applications, scientific publications, manufacturer's specifications, instructions, etc.), whether supra or infra, are hereby incorporated by reference in their entirety. Nothing herein is to be construed as an admission that the present disclosure was not entitled to antedate such disclosure.

In the following, definitions will be provided which apply to all aspects of the present disclosure. The following terms have the following meanings unless otherwise indicated. Any undefined terms have their art recognized meanings.

### Definitions

Terms such as “reduce”, “decrease”, “inhibit” or “impair” as used herein relate to an overall decrease or the ability to cause an overall decrease, preferably of 5% or greater, 10% or greater, 20% or greater, more preferably of 50% or greater, and most preferably of 75% or greater, in the level, e.g. in the level of binding.

Terms such as “increase”, “enhance” or “exceed” preferably relate to an increase or enhancement by about at least 10%, preferably at least 20%, preferably at least 30%, more preferably at least 40%, more preferably at least 50%, even more preferably at least 80%, and most preferably at least 100%, at least 200%, at least 500%, or even more.

The term “plurality” with reference to an object refers to a population of a certain number of said object. In certain embodiments, the term refers to a population of more than 10, 10², 10³, 10⁴, 10⁵, 10⁶, 10⁷, 10⁸, 10⁹, 10¹⁰, 10¹¹, 10¹², 10¹³, 10¹⁴, 10¹⁵, 10¹⁶, 10¹⁷, 10¹⁸, 10¹⁹, 10²⁰, 10²¹, 10²², or 10²³ or more.

Amino acids are the building blocks that form peptides, polypeptides and proteins. The following shows the abbreviations and single letter codes used for amino acids.

| | Abbreviation | Abbreviation |
|---|---|---|
| Full Name | (3 Letter) | (1 Letter) |
| Alanine | Ala | A |
| Arginine | Arg | R |
| Asparagine | Asn | N |
| Aspartate | Asp | D |
| Aspartate or Asparagine | Asx | B |
| Cysteine | Cys | C |
| Glutamate | Glu | E |
| Glutamine | Gln | Q |
| Glutamate or Glutamine | Glx | Z |
| Glycine | Gly | G |
| Histidine | His | H |
| Isoleucine | Ile | I |
| Leucine | Leu | L |
| Lysine | Lys | K |
| Methionine | Met | M |
| Phenylalanine | Phe | F |
| Proline | Pro | P |
| Serine | Ser | S |
| Threonine | Thr | T |
| Tryptophan | Trp | W |
| Tyrosine | Tyr | Y |
| Valine | Val | V |

According to the disclosure, the term “peptide” comprises oligo- and polypeptides and refers to substances which comprise about two or more, about 3 or more, about 4 or more, about 6 or more, about 8 or more, about 10 or more, about 13 or more, about 16 or more, about 20 or more, and up to about 50, about 100 or about 150, consecutive amino acids linked to one another via peptide bonds. The term “protein” or “polypeptide” refers to large peptides, in particular peptides having at least about 151 amino acids, but the terms “peptide”, “protein” and “polypeptide” are used herein usually as synonyms.

A “therapeutic protein” has a positive or advantageous effect on a condition or disease state of a subject when provided to the subject in a therapeutically effective amount. In one embodiment, a therapeutic protein has curative or palliative properties and may be administered to ameliorate, relieve, alleviate, reverse, delay onset of or lessen the severity of one or more symptoms of a disease or disorder. A therapeutic protein may have prophylactic properties and may be used to delay the onset of a disease or to lessen the severity of such disease or pathological condition. The term “therapeutic protein” includes entire proteins or peptides, and can also refer to therapeutically active fragments thereof. It can also include therapeutically active variants of a protein. Examples of therapeutically active proteins include, but are not limited to, antigens for vaccination and cytokines.

“Fragment”, with reference to an amino acid sequence (peptide or protein), relates to a part of an amino acid sequence, i.e., a sequence which represents the amino acid sequence shortened at the N-terminus and/or C-terminus. A fragment shortened at the C-terminus (N-terminal fragment) is obtainable e.g. by translation of a truncated open reading frame that lacks the 3′-end of the open reading frame. A fragment shortened at the N-terminus (C-terminal fragment) is obtainable e.g. by translation of a truncated open reading frame that lacks the 5′-end of the open reading frame, as long as the truncated open reading frame comprises a start codon that serves to initiate translation. A fragment of an amino acid sequence comprises e.g., at least 50%, at least 60%, at least 70%, at least 80%, at least 90% of the amino acid residues from an amino acid sequence. A fragment of an amino acid sequence preferably comprises at least 6, in particular at least 8, at least 12, at least 15, at least 20, at least 30, at least 50, or at least 100 consecutive amino acids from an amino acid sequence.

By “variant” or “variant protein” or “variant polypeptide” herein is meant a protein that differs from a wild type protein by virtue of at least one amino acid modification. The parent polypeptide may be a naturally occurring or wild type (WT) polypeptide, or may be a modified version of a wild type polypeptide. Preferably, the variant polypeptide has at least one amino acid modification compared to the parent polypeptide, e.g. from 1 to about 20 amino acid modifications, and preferably from 1 to about 10 or from 1 to about 5 amino acid modifications compared to the parent.

By “parent polypeptide”, “parent protein”, “precursor polypeptide”, or “precursor protein” as used herein is meant an unmodified polypeptide that is subsequently modified to generate a variant. A parent polypeptide may be a wild type polypeptide, or a variant or engineered version of a wild type polypeptide.

By “wild type” or “WT” or “native” herein is meant an amino acid sequence that is found in nature, including allelic variations, A wild type protein or polypeptide has an amino acid sequence that has not been intentionally modified.

For the purposes of the present disclosure, “variants” of an amino acid sequence (peptide, protein or polypeptide) comprise amino acid insertion variants, amino acid addition variants, amino acid deletion variants and/or amino acid substitution variants. The term “variant” includes all splice variants, posttranslationally modified variants, conformations, isoforms and species homologs, in particular those which are naturally expressed by cells. The term “variant” includes, in particular, fragments of an amino acid sequence.

Amino acid insertion variants comprise insertions of single or two or more amino acids in a particular amino acid sequence. In the case of amino acid sequence variants having an insertion, one or more amino acid residues are inserted into a particular site in an amino acid sequence, although random insertion with appropriate screening of the resulting product is also possible. Amino acid addition variants comprise amino- and/or carboxy-terminal fusions of one or more amino acids, such as 1, 2, 3, 5, 10, 20, 30, 50, or more amino acids. Amino acid deletion variants are characterized by the removal of one or more amino acids from the sequence, such as by removal of 1, 2, 3, 5, 10, 20, 30, 50, or more amino acids. The deletions may be in any position of the protein. Amino acid deletion variants that comprise the deletion at the N-terminal and/or C-terminal end of the protein are also called N-terminal and/or C-terminal truncation variants. Amino acid substitution variants are characterized by at least one residue in the sequence being removed and another residue being inserted in its place. Preference is given to the modifications being in positions in the amino acid sequence which are not conserved between homologous proteins or peptides and/or to replacing amino acids with other ones having similar properties. Preferably, amino acid changes in peptide and protein variants are conservative amino acid changes, i.e., substitutions of similarly charged or uncharged amino acids. A conservative amino acid change involves substitution of one of a family of amino acids which are related in their side chains. Naturally occurring amino acids are generally divided into four families: acidic (aspartate, glutamate), basic (lysine, arginine, histidine), non-polar (alanine, valine, leucine, isoleucine, proline, phenylalanine, methionine, tryptophan), and uncharged polar (glycine, asparagine, glutamine, cysteine, serine, threonine, tyrosine) amino acids. Phenylalanine, tryptophan, and tyrosine are sometimes classified jointly as aromatic amino acids. In one embodiment, conservative amino acid substitutions include substitutions within the following groups:

glycine, alanine;
valine, isoleucine, leucine;
aspartic acid, glutamic acid;
asparagine, glutamine;
serine, threonine;
lysine, arginine; and
phenylalanine, tyrosine.

Preferably the degree of similarity, preferably identity between a given amino acid sequence and an amino acid sequence which is a variant of said given amino acid sequence will be at least about 60%, 65%, 70%, 80%, 81%, 82%, 83%, 84%, 85%, 86%, 87%, 88%, 89%, 90%, 91%, 92%, 93%, 94%, 95%, 96%, 97%, 98%, or 99%. The degree of similarity or identity is given preferably for an amino acid region which is at least about 10%, at least about 20%, at least about 30%, at least about 40%, at least about 50%, at least about 60%, at least about 70%, at least about 80%, at least about 90% or about 100% of the entire length of the reference amino acid sequence. For example, if the reference amino acid sequence consists of 200 amino acids, the degree of similarity or identity is given preferably for at least about 20, at least about 40, at least about 60, at least about 80, at least about 100, at least about 120, at least about 140, at least about 160, at least about 180, or about 200 amino acids, preferably continuous amino acids. In preferred embodiments, the degree of similarity or identity is given for the entire length of the reference amino acid sequence. The alignment for determining sequence similarity, preferably sequence identity can be done with art known tools, preferably using the best sequence alignment, for example, using Align, using standard settings, preferably EMBOSS::needle, Matrix: Blosum62, Gap Open 10.0, Gap Extend 0.5.

“Sequence similarity” indicates the percentage of amino acids that either are identical or that represent conservative amino acid substitutions. “Sequence identity” between two amino acid sequences indicates the percentage of amino acids that are identical between the sequences.

The term “percentage identity” is intended to denote a percentage of amino acid residues which are identical between the two sequences to be compared, obtained after the best alignment, this percentage being purely statistical and the differences between the two sequences being distributed randomly and over their entire length. Sequence comparisons between two amino acid sequences are conventionally carried out by comparing these sequences after having aligned them optimally, said comparison being carried out by segment or by “window of comparison” in order to identify and compare local regions of sequence similarity. The optimal alignment of the sequences for comparison may be produced, besides manually, by means of the local homology algorithm of Smith and Waterman, 1981, Ads App. Math. 2, 482, by means of the local homology algorithm of Neddleman and Wunsch, 1970, J. Mol. Biol. 48, 443, by means of the similarity search method of Pearson and Lipman, 1988, Proc. Natl Acad. Sci. USA 85, 2444, or by means of computer programs which use these algorithms (GAP, BESTFIT, FASTA, BLAST P, BLAST N and TFASTA in Wisconsin Genetics Software Package, Genetics Computer Group, 575 Science Drive, Madison, Wis.).

The percentage identity is calculated by determining the number of identical positions between the two sequences being compared, dividing this number by the number of positions compared and multiplying the result obtained by 100 so as to obtain the percentage identity between these two sequences.

Homologous amino acid sequences exhibit according to the disclosure at least 40%, in particular at least 50%, at least 60%, at least 70%, at least 80%, at least 90% and preferably at least 95%, at least 98 or at least 99% identity of the amino acid residues.

The amino acid sequence variants described herein may readily be prepared by the skilled person, for example, by recombinant DNA manipulation. The manipulation of DNA sequences for preparing peptides or proteins having substitutions, additions, insertions or deletions, is described in detail in Sambrook et al. (1989), for example. Furthermore, the peptides and amino acid variants described herein may be readily prepared with the aid of known peptide synthesis techniques such as, for example, by solid phase synthesis and similar methods.

In one embodiment, a fragment or variant of an amino acid sequence (peptide or protein) is preferably a “functional fragment” or “functional variant”. The term “functional fragment” or “functional variant” of an amino acid sequence relates to any fragment or variant exhibiting one or more functional properties identical or similar to those of the amino acid sequence from which it is derived, i.e., it is functionally equivalent. With respect to antigens, one particular function is one or more immunostimulating activities displayed by the amino acid sequence from which the fragment or variant is derived and/or binding to the receptor(s) the amino acid sequence from which the fragment or variant is derived binds to. The term “functional fragment” or “functional variant”, as used herein, in particular refers to a variant molecule or sequence that comprises an amino acid sequence that is altered by one or more amino acids compared to the amino acid sequence of the parent molecule or sequence and that is still capable of fulfilling one or more of the functions of the parent molecule or sequence, e.g., binding to a target molecule. In one embodiment, the modifications in the amino acid sequence of the parent molecule or sequence do not significantly affect or alter the binding characteristics of the molecule or sequence. In different embodiments, binding of the functional fragment or functional variant may be reduced but still significantly present, e.g., binding of the functional variant may be at least 50%, at least 60%, at least 70%, at least 80%, or at least 90% of the parent molecule or sequence. However, in other embodiments, binding of the functional fragment or functional variant may be enhanced compared to the parent molecule or sequence.

An amino acid sequence (peptide, protein or polypeptide) “derived from” a designated amino acid sequence (peptide, protein or polypeptide) refers to the origin of the first amino acid sequence. Preferably, the amino acid sequence which is derived from a particular amino acid sequence has an amino acid sequence that is identical, essentially identical or homologous to that particular sequence or a fragment thereof. Amino acid sequences derived from a particular amino acid sequence may be variants of that particular sequence or a fragment thereof. For example, it will be understood by one of ordinary skill in the art that the antigens suitable for use herein may be altered such that they vary in sequence from the naturally occurring or native sequences from which they were derived, while retaining the desirable activity of the native sequences.

As used herein, an “instructional material” or “instructions” includes a publication, a recording, a diagram, or any other medium of expression which can be used to communicate the usefulness of the compositions and methods of the invention. The instructional material of the kit of the invention may, for example, be affixed to a container which contains the compositions of the invention or be shipped together with a container which contains the compositions. Alternatively, the instructional material may be shipped separately from the container with the intention that the instructional material and the compositions be used cooperatively by the recipient.

“Isolated” means altered or removed from the natural state. For example, a nucleic acid or a peptide naturally present in a living animal is not “isolated”, but the same nucleic acid or peptide partially or completely separated from the coexisting materials of its natural state is “isolated”. An isolated nucleic acid or protein can exist in substantially purified form, or can exist in a non-native environment such as, for example, a host cell.

The term “recombinant” in the context of the present invention means “made through genetic engineering”. Preferably, a “recombinant object” such as a recombinant cell in the context of the present invention is not occurring naturally.

The term “naturally occurring” as used herein refers to the fact that an object can be found in nature. For example, a peptide or nucleic acid that is present in an organism (including viruses) and can be isolated from a source in nature and which has not been intentionally modified by man in the laboratory is naturally occurring.

A “lentivirus” as used herein refers to a genus of the Retroviridae family. Lentiviruses are unique among the retroviruses in being able to infect non-dividing cells; they can deliver a significant amount of genetic information into the DNA of the host cell, so they are one of the most efficient methods of a gene delivery vector. HIV, SIV, and FIV are all examples of lentiviruses. Vectors derived from lentiviruses offer the means to achieve significant levels of gene transfer in vivo.

By the term “specifically binds”, as used herein, is meant a molecule such as an antibody or CAR which recognizes a specific antigen, but does not substantially recognize or bind other molecules in a sample or in a subject. For example, an antibody that specifically binds to an antigen from one species may also bind to that antigen from one or more other species. But, such cross-species reactivity does not itself alter the classification of an antibody as specific. In another example, an antibody that specifically binds to an antigen may also bind to different allelic forms of the antigen. However, such cross reactivity does not itself alter the classification of an antibody as specific. In some instances, the terms “specific binding” or “specifically binding”, can be used in reference to the interaction of an antibody, a protein, or a peptide with a second chemical species, to mean that the interaction is dependent upon the presence of a particular structure (e.g., an antigenic determinant or epitope) on the chemical species; for example, an antibody recognizes and binds to a specific protein structure rather than to proteins generally. If an antibody is specific for epitope “A”, the presence of a molecule containing epitope A (or free, unlabeled A), in a reaction containing labeled “A” and the antibody, will reduce the amount of labeled A bound to the antibody.

The term “genetic modification” includes the transfection of cells with nucleic acid. The term “transfection” relates to the introduction of nucleic acids, in particular RNA, into a cell. For purposes of the present invention, the term “transfection” also includes the introduction of a nucleic acid into a cell or the uptake of a nucleic acid by such cell, wherein the cell may be present in a subject, e.g., a patient. Thus, according to the present invention, a cell for transfection of a nucleic acid described herein can be present in vitro or in vivo, e.g. the cell can form part of an organ, a tissue and/or an organism of a patient. According to the invention, transfection can be transient or stable. For some applications of transfection, it is sufficient if the transfected genetic material is only transiently expressed. RNA can be transfected into cells to transiently express its coded protein. Since the nucleic acid introduced in the transfection process is usually not integrated into the nuclear genome, the foreign nucleic acid will be diluted through mitosis or degraded. Cells allowing episomal amplification of nucleic acids greatly reduce the rate of dilution. If it is desired that the transfected nucleic acid actually remains in the genome of the cell and its daughter cells, a stable transfection must occur. Such stable transfection can occur if the nucleic acid introduced in the transfection process is integrated into the nuclear genome and can be achieved, for example, by using virus-based systems or transposon-based systems for transfection. Generally, cells that are genetically modified to express an antigen receptor are stably transfected with nucleic acid encoding the antigen receptor, while, generally, nucleic acid encoding antigen is transiently transfected into cells.

### Immune Effector Cells

The cells used in connection with the present invention and into which nucleic acids (DNA or RNA) encoding antigen receptors may be introduced include, in particular, immune effector cells such as cells with lytic potential, in particular lymphoid cells, and are preferably T cells, in particular cytotoxic lymphocytes, preferably selected from cytotoxic T cells, natural killer (NK) cells, and lymphokine-activated killer (LAK) cells. Upon activation, each of these cytotoxic lymphocytes triggers the destruction of target cells. For example, cytotoxic T cells trigger the destruction of target cells by either or both of the following means. First, upon activation T cells release cytotoxins such as perforin, granzymes, and granulysin. Perforin and granulysin create pores in the target cell, and granzymes enter the cell and trigger a caspase cascade in the cytoplasm that induces apoptosis (programmed cell death) of the cell. Second, apoptosis can be induced via Fas-Fas ligand interaction between the T cells and target cells. The cells used in connection with the present invention will preferably be autologous cells, although heterologous cells or allogenic cells can be used.

The term “effector functions” in the context of the present invention includes any functions mediated by components of the immune system that result, for example, in the killing of diseased cells such as tumor cells, or in the inhibition of tumor growth and/or inhibition of tumor development, including inhibition of tumor dissemination and metastasis. Preferably, the effector functions in the context of the present invention are T cell mediated effector functions. Such functions comprise in the case of a helper T cell (CD4⁺ T cell) the release of cytokines and/or the activation of CD8⁺ lymphocytes (CTLs) and/or B cells, and in the case of CTL the elimination of cells, i.e., cells characterized by expression of an antigen, for example, via apoptosis or perforin-mediated cell lysis, production of cytokines such as IFN-γ and TNF-α, and specific cytolytic killing of antigen expressing target cells.

The term “immune effector cell” or “immunoreactive cell” in the context of the present invention relates to a cell which exerts effector functions during an immune reaction. An “immune effector cell” in one embodiment is capable of binding an antigen such as an antigen presented in the context of MHC on a cell or expressed on the surface of a cell and mediating an immune response. For example, immune effector cells comprise T cells (cytotoxic T cells, helper T cells, tumor infiltrating T cells), B cells, natural killer cells, neutrophils, macrophages, and dendritic cells. Preferably, in the context of the present invention, “immune effector cells” are T cells, preferably CD4⁺ and/or CD8⁺ T cells, most preferably CD8⁺ T cells. According to the invention, the term “immune effector cell” also includes a cell which can mature into an immune cell (such as T cell, in particular T helper cell, or cytolytic T cell) with suitable stimulation. Immune effector cells comprise CD34⁺ hematopoietic stem cells, immature and mature T cells and immature and mature B cells. The differentiation of T cell precursors into a cytolytic T cell, when exposed to an antigen, is similar to clonal selection of the immune system.

Preferably, an “immune effector cell” recognizes an antigen with some degree of specificity, in particular if presented in the context of MHC or present on the surface of diseased cells such as cancer cells. Preferably, said recognition enables the cell that recognizes an antigen to be responsive or reactive. If the cell is a helper T cell (CD4⁺ T cell) such responsiveness or reactivity may involve the release of cytokines and/or the activation of CD8⁺ lymphocytes (CTLs) and/or B cells. If the cell is a CTL such responsiveness or reactivity may involve the elimination of cells, i.e., cells characterized by expression of an antigen, for example, via apoptosis or perforin-mediated cell lysis. According to the invention, CTL responsiveness may include sustained calcium flux, cell division, production of cytokines such as IFN-γ and TNF-α, up-regulation of activation markers such as CD44 and CD69, and specific cytolytic killing of antigen expressing target cells. CTL responsiveness may also be determined using an artificial reporter that accurately indicates CTL responsiveness. Such CTL that recognizes an antigen and are responsive or reactive are also termed “antigen-responsive CTL” herein.

In one embodiment, the genetically modified immune effector cells are CAR-expressing immune effector cells. In one embodiment, the genetically modified immune effector cells are TCR-expressing immune effector cells,

The immune effector cells to be used according to the invention may express an endogenous antigen receptor such as T cell receptor or B cell receptor or may lack expression of an endogenous antigen receptor.

A “lymphoid cell” is a cell which, optionally after suitable modification, e.g. after transfer of an antigen receptor such as a TCR or a CAR, is capable of producing an immune response such as a cellular immune response, or a precursor cell of such cell, and includes lymphocytes, preferably T lymphocytes, lymphoblasts, and plasma cells. A lymphoid cell may be an immune effector cell as described herein. A preferred lymphoid cell is a T cell which can be modified to express an antigen receptor on the cell surface. In one embodiment, the lymphoid cell lacks endogenous expression of a T cell receptor.

The terms “T cell” and “T lymphocyte” are used interchangeably herein and include T helper cells (CD4⁺ T cells) and cytotoxic T cells (CTLs, CD8⁺ T cells) which comprise cytolytic T cells. The term “antigen-specific T cell” or similar terms relate to a T cell which recognizes the antigen to which the T cell is targeted and preferably exerts effector functions of T cells. T cells are considered to be specific for antigen if the cells kill target cells expressing an antigen. T cell specificity may be evaluated using any of a variety of standard techniques, for example, within a chromium release assay or proliferation assay. Alternatively, synthesis of lymphokines (such as interferon-γ) can be measured.

T cells belong to a group of white blood cells known as lymphocytes, and play a central role in cell-mediated immunity. They can be distinguished from other lymphocyte types, such as B cells and natural killer cells by the presence of a special receptor on their cell surface called T cell receptors (TCR). The thymus is the principal organ responsible for the maturation of T cells. Several different subsets of T cells have been discovered, each with a distinct function.

T helper cells assist other white blood cells in immunologic processes, including maturation of B cells into plasma cells and activation of cytotoxic T cells and macrophages, among other functions. These cells are also known as CD4⁺ T cells because they express the CD4 glycoprotein on their surface. Helper T cells become activated when they are presented with peptide antigens by MHC class II molecules that are expressed on the surface of antigen presenting cells (APCs). Once activated, they divide rapidly and secrete small proteins called cytokines that regulate or assist in the active immune response.

Cytotoxic T cells destroy virally infected cells and tumor cells, and are also implicated in transplant rejection. These cells are also known as CD8⁺ T cells since they express the CD8 glycoprotein on their surface. These cells recognize their targets by binding to antigen associated with MHC class I, which is present on the surface of nearly every cell of the body.

“Regulatory T cells” or “Tregs” are a subpopulation of T cells that modulate the immune system, maintain tolerance to self-antigens, and prevent autoimmune disease. Tregs are immunosuppressive and generally suppress or downregulate induction and proliferation of effector T cells. Tregs express the biomarkers CD4, FoxP3, and CD25.

As used herein, the term “naïve T cell” refers to mature T cells that, unlike activated or memory T cells, have not encountered their cognate antigen within the periphery. Naïve T cells are commonly characterized by the surface expression of L-selectin (CD62L), the absence of the activation markers CD25, CD44 or CD69 and the absence of the memory CD45R0 isoform.

As used herein, the term “memory T cells” refers to a subgroup or subpopulation of T cells that have previously encountered and responded to their cognate antigen. At a second encounter with the antigen, memory T cells can reproduce to mount a faster and stronger immune response than the first time the immune system responded to the antigen. Memory T cells may be either CD4⁺ or CD8⁺ and usually express CD45RO.

According to the invention, the term “T cell” also includes a cell which can mature into a T cell with suitable stimulation.

A majority of T cells have a T cell receptor (TCR) existing as a complex of several proteins. The actual T cell receptor is composed of two separate peptide chains, which are produced from the independent T cell receptor alpha and beta (TCRα and TCRβ) genes and are called α- and β-TCR chains. Γδ T cells (gamma delta T cells) represent a small subset of T cells that possess a distinct T cell receptor (TCR) on their surface. However, in γδ T cells, the TCR is made up of one γ-chain and one δ-chain. This group of T cells is much less common (2% of total T cells) than the αβ T cells.

All T cells originate from hematopoietic stem cells in the bone marrow. Hematopoietic progenitors derived from hematopoietic stem cells populate the thymus and expand by cell division to generate a large population of immature thymocytes. The earliest thymocytes express neither CD4 nor CD8, and are therefore classed as double-negative (CD4⁻CD8⁻) cells. As they progress through their development they become double-positive thymocytes (CD4⁺CD8⁻), and finally mature to single-positive (CD4⁺CD8⁻ or CD4⁻CD8⁺) thymocytes that are then released from the thymus to peripheral tissues.

T cells may generally be prepared in vitro or ex vivo, using standard procedures. For example, T cells may be isolated from bone marrow, peripheral blood or a fraction of bone marrow or peripheral blood of a mammal, such as a patient, using a commercially available cell separation system. Alternatively, T cells may be derived from related or unrelated humans, non-human animals, cell lines or cultures. A sample comprising T cells may, for example, be peripheral blood mononuclear cells (PBMC).

As used herein, the term “NK cell” or “Natural Killer cell” refers to a subset of peripheral blood lymphocytes defined by the expression of CD56 or CD16 and the absence of the T cell receptor. As provided herein, the NK cell can also be differentiated from a stem cell or progenitor cell.

### Genetic Modification to Express Antigen Receptors

Cells described herein such as immune effector cells are genetically modified ex vivo/in vitro or in vivo in a subject being treated to express an antigen receptor such as a chimeric antigen receptor (CAR) or a T cell receptor (TCR) binding antigen or a procession product thereof, in particular when present on or presented by a target cell, e.g., an antigen presenting cell or a diseased cell. In one embodiment, modification to express an antigen receptor takes place ex vivo/in vitro. Subsequently, modified cells may be administered to a patient. In one embodiment, modification to express an antigen receptor takes place in vivo. The cells may be endogenous cells of the patient or may have been administered to a patient.

### Chimeric Antigen Receptors

Adoptive cell transfer therapy with CAR-engineered T cells expressing chimeric antigen receptors is a promising anti-cancer therapeutic as CAR-modified T cells can be engineered to target virtually any tumor antigen. For example, patient's T cells may be genetically engineered (genetically modified) to express CARs specifically directed towards antigens on the patient's tumor cells, then infused back into the patient.

According to the invention, the term “CAR” (or “chimeric antigen receptor”) is synonymous with the terms “chimeric T cell receptor” and “artificial T cell receptor” and relates to an artificial receptor comprising a single molecule or a complex of molecules which recognizes, i.e. binds to, a target structure (e.g. an antigen) on a target cell such as a cancer cell (e.g. by binding of an antigen binding domain to an antigen expressed on the surface of the target cell) and may confer specificity onto an immune effector cell such as a T cell expressing said CAR on the cell surface. Such cells do not necessarily require processing and presentation of an antigen for recognition of the target cell but rather may recognize preferably with specificity any antigen present on a target cell. Preferably, recognition of the target structure by a CAR results in activation of an immune effector cell expressing said CAR. A CAR may comprise one or more protein units said protein units comprising one or more domains as described herein. The term “CAR” does not include T cell receptors.

A CAR comprises a target-specific binding element otherwise referred to as an antigen binding moiety or antigen binding domain that is generally part of the extracellular domain of the CAR. The antigen binding domain recognizes a ligand that acts as a cell surface marker on target cells associated with a particular disease state. Specifically, the CAR of the invention targets the antigen such as tumor antigen on a diseased cell such as tumor cell.

In one embodiment, the binding domain in the CAR binds specifically to the antigen. In one embodiment, the antigen to which the binding domain in the CAR binds is expressed in a cancer cell (tumor antigen). In one embodiment, the antigen is expressed on the surface of a cancer cell. In one embodiment, the binding domain binds to an extracellular domain or to an epitope in an extracellular domain of the antigen. In one embodiment, the binding domain binds to native epitopes of the antigen present on the surface of living cells.

In one embodiment of the invention, an antigen binding domain comprises a variable region of a heavy chain of an immunoglobulin (VH) with a specificity for the antigen and a variable region of a light chain of an immunoglobulin (VL) with a specificity for the antigen. In one embodiment, an immunoglobulin is an antibody. In one embodiment, said heavy chain variable region (VH) and the corresponding light chain variable region (VL) are connected via a peptide linker. Preferably, the antigen binding moiety portion in the CAR is a scFv,

The CAR is designed to comprise a transmembrane domain that is fused to the extracellular domain of the CAR. In one embodiment, the transmembrane domain is not naturally associated with one of the domains in the CAR. In one embodiment, the transmembrane domain is naturally associated with one of the domains in the CAR. In one embodiment, the transmembrane domain is modified by amino acid substitution to avoid binding of such domains to the transmembrane domains of the same or different surface membrane proteins to minimize interactions with other members of the receptor complex. The transmembrane domain may be derived either from a natural or from a synthetic source. Where the source is natural, the domain may be derived from any membrane-bound or transmembrane protein. Transmembrane regions of particular use in this invention may be derived from (i.e. comprise at least the transmembrane region(s) of) the alpha, beta or zeta chain of the T cell receptor, CD28, CD3 epsilon, CD45, CD4, CD5, CD8, CD9, CD16, CD22, CD33, CD37, CD64, CD80, CD86, CD134, CD137, CD154. Alternatively the transmembrane domain may be synthetic, in which case it will comprise predominantly hydrophobic residues such as leucine and valine. Preferably a triplet of phenylalanine, tryptophan and valine will be found at each end of a synthetic transmembrane domain.

In some instances, the CAR of the invention comprises a hinge domain which forms the linkage between the transmembrane domain and the extracellular domain.

The cytoplasmic domain or otherwise the intracellular signaling domain of the CAR is responsible for activation of at least one of the normal effector functions of the immune cell in which the CAR has been placed in. The term “effector function” refers to a specialized function of a cell. Effector function of a T cell, for example, may be cytolytic activity or helper activity including the secretion of cytokines. Thus the term “intracellular signaling domain” refers to the portion of a protein which transduces the effector function signal and directs the cell to perform a specialized function. While usually the entire intracellular signaling domain can be employed, in many cases it is not necessary to use the entire chain. To the extent that a truncated portion of the intracellular signaling domain is used, such truncated portion may be used in place of the intact chain as long as it transduces the effector function signal. The term intracellular signaling domain is thus meant to include any truncated portion of the intracellular signaling domain sufficient to transduce the effector function signal.

It is known that signals generated through the TCR alone are insufficient for full activation of the T cell and that a secondary or co-stimulatory signal is also required. Thus, T cell activation can be said to be mediated by two distinct classes of cytoplasmic signaling sequence: those that initiate antigen-dependent primary activation through the TCR (primary cytoplasmic signaling sequences) and those that act in an antigen-independent manner to provide a secondary or co-stimulatory signal (secondary cytoplasmic signaling sequences).

In one embodiment, the CAR comprises a primary cytoplasmic signaling sequence derived from CD3-zeta. Further, the cytoplasmic domain of the CAR may comprise the CD3-zeta signaling domain combined with a costimulatory signaling region.

The identity of the co-stimulation domain is limited only in that it has the ability to enhance cellular proliferation and survival upon binding of the targeted moiety by the CAR. Suitable co-stimulation domains include CD28, CD137 (4-1 BB), a member of the tumor necrosis factor receptor (TNFR) superfamily, CD134 (OX40), a member of the TNFR-superfamily of receptors, and CD278 (ICOS), a CD28-superfamily co-stimulatory molecule expressed on activated T cells. The skilled person will understand that sequence variants of these noted co-stimulation domains can be used without adversely impacting the invention, where the variants have the same or similar activity as the domain on which they are modeled. Such variants will have at least about 80% sequence identity to the amino acid sequence of the domain from which they are derived. In some embodiments of the invention, the CAR constructs comprise two co-stimulation domains. While the particular combinations include all possible variations of the four noted domains, specific examples include CD28+CD137 (4-1BB) and CD28+CD134 (OX40).

The cytoplasmic signaling sequences within the cytoplasmic signaling portion of the CAR may be linked to each other in a random or specified order. Optionally, a short oligo- or polypeptide linker, preferably between 2 and 10 amino acids in length may form the linkage. A glycine-serine doublet provides a particularly suitable linker.

In one embodiment, the CAR comprises a signal peptide which directs the nascent protein into the endoplasmic reticulum. In one embodiment, the signal peptide precedes the antigen binding domain. In one embodiment, the signal peptide is derived from an immunoglobulin such as IgG.

A CAR may comprise the above domains, together in the form of a fusion protein. Such fusion proteins will generally comprise an antigen binding domain, one or more co-stimulation domains, and a signaling sequence, linked in a N-terminal to C-terminal direction. However, the CARs of the present invention are not limited to this arrangement and other arrangements are acceptable and include a binding domain, a signaling domain, and one or more co-stimulation domains. It will be understood that because the binding domain must be free to bind antigen, the placement of the binding domain in the fusion protein will generally be such that display of the region on the exterior of the cell is achieved. In the same manner, because the co-stimulation and signaling domains serve to induce activity and proliferation of the cytotoxic lymphocytes, the fusion protein will generally display these two domains in the interior of the cell.

In one embodiment, a CAR molecule comprises:

i) a target antigen (e.g., CLDN6 or CLDN18.2) binding domain;
ii) a transmembrane domain; and
iii) an intracellular domain that comprises a 4-1BB costimulatory domain, and a CD3-zeta signaling domain.

In one embodiment, the antigen binding domain comprises an scFv. In one embodiment, the transmembrane domain comprises a transmembrane domain of a protein selected from the group consisting of the alpha, beta or zeta chain of the T cell receptor, CD28, CD3 epsilon, CD45, CD4, CD5, CD8, CD9, CD16, CD22, CD33, CD37, CD64, CD80, CD86, CD134, CD154, KIRDS2, OX40, CD2, CD27, LFA-1 (CD11a, CD18), ICOS (CD278), 4-1BB (CD137), GITR, CD40, BAFFR, HVEM (LIGHTR), SLAMF7, NKp80 (KLRF1), CD160, CD19, IL2R beta, IL2R gamma, IL7Ra, ITGA1, VLA1, CD49a, ITGA4, IA4, CD49D, ITGA6, VLA-6, CD49f, ITGAD, CDIId, ITGAE, CD103, ITGAL, CDIIa, LFA-1, ITGAM, CDIIb, ITGAX, CDIIc, ITGBI, CD29, ITGB2, CD18, LFA-1, ITGB7, TNFR2, DNAMI (CD226), SLAMF4 (CD244, 2B4), CD84, CD96 (Tactile), CEACAM1, CRT AM, Ly9 (CD229), CD160 (BY55), PSGLI, CDIOO (SEMA4D), SLAMF6 (NTB-A, LyI08), SLAM (SLAMF1, CD150, IPO-3), BLAME (SLAMF8), SELPLG (CD162), LTBR, PAG/Cbp, NKp44, NKp30, NKp46, NKG2D, and NKG2C, or a functional variant thereof. In one embodiment, the transmembrane domain comprises a CD8a transmembrane domain. In one embodiment, the antigen binding domain is connected to the transmembrane domain by a hinge domain. In one embodiment, the hinge domain is a CD8a hinge domain.

In one embodiment, the CAR molecule of the invention comprises:

i) a target antigen binding domain;
ii) a CD8a hinge domain;
iii) a CD8a transmembrane domain; and
iv) an intracellular domain that comprises a 4-1BB costimulatory domain, and a CD3-zeta signaling domain.

The term “antibody” includes an immunoglobulin comprising at least two heavy (H) chains and two light (L) chains inter-connected by disulfide bonds. Each heavy chain is comprised of a heavy chain variable region (abbreviated herein as VH) and a heavy chain constant region. Each light chain is comprised of a light chain variable region (abbreviated herein as VL) and a light chain constant region. The VH and VL regions can be further subdivided into regions of hypervariability, termed complementarity determining regions (CDR), interspersed with regions that are more conserved, termed framework regions (FR). Each VH and VL is composed of three CDRs and four FRs, arranged from amino-terminus to carboxy-terminus in the following order: FR1, CDR1, FR2, CDR2, FR3, CDR3, FR4. The variable regions of the heavy and light chains contain a binding domain that interacts with an antigen. The constant regions of the antibodies may mediate the binding of the immunoglobulin to host tissues or factors, including various cells of the immune system (e.g., effector cells) and the first component (Clq) of the classical complement system. An antibody binds, preferably specifically binds with an antigen. Antibodies can be intact immunoglobulins derived from natural sources or from recombinant sources and can be immunoreactive portions or fragments of intact immunoglobulins. Antibodies are typically tetramers of immunoglobulin molecules. The antibodies in the present invention may exist in a variety of forms including, for example, polyclonal antibodies, monoclonal antibodies, Fv, Fab and F(ab′)₂, as well as single chain antibodies and humanized antibodies (Harlow et al., 1999, In: Using Antibodies: A Laboratory Manual, Cold Spring Harbor Laboratory Press, NY; Harlow et al., 1989, in: Antibodies: A Laboratory Manual, Cold Spring Harbor, N.Y.; Houston et al., 1988, Proc. Natl. Acad. Sci. USA 85:5879-5883; Bird et al., 1988, Science 242:423-426).

Antibodies expressed by B cells are sometimes referred to as the BCR (B cell receptor) or antigen receptor. The five members included in this class of proteins are IgA, IgG, IgM, IgD, and IgE. IgA is the primary antibody that is present in body secretions, such as saliva, tears, breast milk, gastrointestinal secretions and mucus secretions of the respiratory and genitourinary tracts. IgG is the most common circulating antibody. IgM is the main immunoglobulin produced in the primary immune response in most subjects. It is the most efficient immunoglobulin in agglutination, complement fixation, and other antibody responses, and is important in defense against bacteria and viruses. IgD is the immunoglobulin that has no known antibody function, but may serve as an antigen receptor. IgE is the immunoglobulin that mediates immediate hypersensitivity by causing release of mediators from mast cells and basophils upon exposure to allergen.

The term “antibody fragment” refers to a portion of an intact antibody and typically comprises the antigenic determining variable regions of an intact antibody.

Examples of antibody fragments include, but are not limited to, Fab, Fab′, F(ab′)₂, and Fv fragments, linear antibodies, scFv antibodies, and multispecific antibodies formed from antibody fragments.

An “antibody heavy chain”, as used herein, refers to the larger of the two types of polypeptide chains present in antibody molecules in their naturally occurring conformations.

An “antibody light chain”, as used herein, refers to the smaller of the two types of polypeptide chains present in antibody molecules in their naturally occurring conformations, K and A light chains refer to the two major antibody light chain isotypes.

According to the disclosure, a CAR which when present on a T cell recognizes an antigen such as on the surface of antigen presenting cells or diseased cells such as cancer cells, such that the T cell is stimulated, and/or expanded or exerts effector functions as described above.

### Genetic Modification of Immune Effector Cells

Particles described herein that are functionalized with a DARPin as described herein for specific targeting of immune effector cells, in particular CD8⁺ T cells, may be used ex vivo/in vitro or in vivo for delivering nucleic acid encoding antigen receptors to immune effector cells such as T cells to produce cells genetically modified to express the antigen receptors. Such genetic modification includes non-viral-based DNA transfection, non-viral-based RNA transfection, e.g., mRNA transfection, transposon-based systems, and viral-based systems. Non-viral-based DNA transfection has low risk of insertional mutagenesis. Transposon-based systems can integrate transgenes more efficiently than plasmids that do not contain an integrating element. Viral-based systems include the use of γ-retroviruses and lentiviral vectors. γ-Retroviruses are relatively easy to produce, efficiently and permanently transduce T cells, and have preliminarily proven safe from an integration standpoint in primary human T cells. Lentiviral vectors also efficiently and permanently transduce T cells but are more expensive to manufacture. They are also potentially safer than retrovirus based systems.

In one embodiment of all aspects of the invention, T cells or T cell progenitors are transfected either ex vivo or in vivo with nucleic acid encoding the antigen receptor. In one embodiment, a combination of ex vivo and in vivo transfection may be used. In one embodiment of all aspects of the invention, the T cells or T cell progenitors are from the subject to be treated. In one embodiment of all aspects of the invention, the T cells or T cell progenitors are from a subject which is different to the subject to be treated.

In one aspect of the invention, CART cells may be produced in vivo, and therefore nearly instantaneously, using particles such as nanoparticles described herein targeted to T cells. For example, lipid and/or polymer-based nanoparticles may be coupled to CD8-specific DARPins for binding to CD8 on T cells. Upon binding to T cells, these particles are endocytosed. Their contents, for example nucleic acid encoding antigen receptor, e.g., plasmid DNA encoding an anti-tumor antigen CAR, may be directed to the T cell nucleus due to, for example, the inclusion of peptides containing microtubule-associated sequences (MTAS) and nuclear localization signals (NLSs). The inclusion of transposons flanking the nucleic acid encoding antigen receptor, e.g., the CAR gene expression cassette, and a separate nucleic acid, e.g., plasmid, encoding a hyperactive transposase, may allow for the efficient integration of the nucleic acid encoding antigen receptor, e.g., the CAR vector, into chromosomes.

Another possibility is to use the CRISPR/Cas9 method to deliberately place an antigen receptor coding sequence such as a CAR coding sequence at a specific locus. For example, existing T cell receptors (TCR) may be knocked out, while knocking in the CAR and placing it under the dynamic regulatory control of the endogenous promoter that would otherwise moderate TCR expression.

Accordingly, besides nucleic acid encoding an antigen receptor the particles described herein may also deliver as cargo gene editing tools like CRISPR/Cas9 (or related) or transposon systems like sleeping beauty or piggy bag. Such tools (e.g. transposase, gene editing tools like CRISPR/Cas9) for genomic integration/editing may be delivered as protein or coding nucleic acid (DNA or RNA). Nevertheless, also delivery of mRNA is an option to induce transient expression of antigen receptors like CARs or T-cell receptors (TCR).

In one embodiment of all aspects of the invention, the cells genetically modified to express an antigen receptor are stably or transiently transfected with nucleic acid encoding the antigen receptor. Thus, the nucleic acid encoding the antigen receptor is integrated or not integrated into the genome of the cells.

In one embodiment of all aspects of the invention, the cells genetically modified to express an antigen receptor are inactivated for expression of an endogenous T cell receptor and/or endogenous HLA.

In one embodiment of all aspects of the invention, the cells described herein may be autologous, allogeneic or syngeneic to the subject to be treated. In one embodiment, the present disclosure envisions the removal of cells from a patient and the subsequent re-delivery of the cells to the patient. In one embodiment, the present disclosure does not envision the removal of cells from a patient. In the latter case all steps of genetic modification of cells are performed in vivo.

The term “autologous” is used to describe anything that is derived from the same subject. For example, “autologous transplant” refers to a transplant of tissue or organs derived from the same subject. Such procedures are advantageous because they overcome the immunological barrier which otherwise results in rejection.

The term “allogeneic” is used to describe anything that is derived from different individuals of the same species. Two or more individuals are said to be allogeneic to one another when the genes at one or more loci are not identical,

The term “syngeneic” is used to describe anything that is derived from individuals or tissues having identical genotypes, i.e., identical twins or animals of the same inbred strain, or their tissues.

The term “heterologous” is used to describe something consisting of multiple different elements. As an example, the transfer of one individual's bone marrow into a different individual constitutes a heterologous transplant. A heterologous gene is a gene derived from a source other than the subject.

### Nucleic Acid Containing Particles

In the context of the present disclosure, the term “particle” relates to a structured entity formed by molecules or molecule complexes. In one embodiment, the term “particle” relates to a micro- or nano-sized structure, such as a micro- or nano-sized compact structure dispersed in a medium. In one embodiment, a particle is a nucleic acid containing particle such as a particle comprising DNA, RNA or a mixture thereof.

Electrostatic interactions between positively charged molecules such as polymers and lipids and negatively charged nucleic acid are involved in particle formation. This results in complexation and spontaneous formation of nucleic acid particles. In one embodiment, a nucleic acid particle is a nanoparticle.

As used in the present disclosure, “nanoparticle” refers to a particle having an average diameter suitable for parenteral administration.

A “nucleic acid particle” can be used to deliver nucleic acid to a target site of interest (e.g., cell, tissue, organ, and the like). A nucleic acid particle may be formed from at least one cationic or cationically ionizable lipid or lipid-like material such as DOTAP, at least one cationic polymer such as protamine, or a mixture thereof and nucleic acid. Nucleic acid particles include lipid nanoparticle (LNP)-based and lipoplex (LPX)-based formulations.

Without intending to be bound by any theory, it is believed that the cationic or cationically ionizable lipid or lipid-like material and the cationic polymer combine together with the nucleic acid to form aggregates, and this aggregation results in colloidally stable particles.

In one embodiment, particles described herein further comprise at least one lipid or lipid-like material other than a cationic or cationically ionizable lipid or lipid-like material, at least one polymer other than a cationic polymer, or a mixture thereof

In some embodiments, nucleic acid particles comprise more than one type of nucleic acid molecules, where the molecular parameters of the nucleic acid molecules may be similar or different from each other, like with respect to molar mass or fundamental structural elements such as molecular architecture, capping, coding regions or other features,

Nucleic acid particles described herein may have an average diameter that in one embodiment ranges from about 30 nm to about 1000 nm, from about 50 nm to about 800 nm, from about 70 nm to about 600 nm, from about 90 nm to about 400 nm, or from about 100 nm to about 300 nm.

Nucleic acid particles described herein, e.g. generated by the processes described herein, exhibit a polydispersity index less than about 0.5, less than about 0.4, less than about 0.3, or about 0.2 or less. By way of example, the nucleic acid particles can exhibit a polydispersity index in a range of about 0.1 to about 0.3 or about 0.2 to about 0.3.

Nucleic acid particles described herein can be prepared using a wide range of methods that may involve obtaining a colloid from at least one cationic or cationically ionizable lipid or lipid-like material and/or at least one cationic polymer and mixing the colloid with nucleic acid to obtain nucleic acid particles.

The term “colloid” as used herein relates to a type of homogeneous mixture in which dispersed particles do not settle out. The insoluble particles in the mixture are microscopic, with particle sizes between 1 and 1000 nanometers. The mixture may be termed a colloid or a colloidal suspension. Sometimes the term “colloid” only refers to the particles in the mixture and not the entire suspension.

For the preparation of colloids comprising at least one cationic or cationically ionizable lipid or lipid-like material and/or at least one cationic polymer methods are applicable herein that are conventionally used for preparing liposomal vesicles and are appropriately adapted. The most commonly used methods for preparing liposomal vesicles share the following fundamental stages: (i) lipids dissolution in organic solvents, (ii) drying of the resultant solution, and (iii) hydration of dried lipid (using various aqueous media).

In the film hydration method, lipids are firstly dissolved in a suitable organic solvent, and dried down to yield a thin film at the bottom of the flask. The obtained lipid film is hydrated using an appropriate aqueous medium to produce a liposomal dispersion. Furthermore, an additional downsizing step may be included.

Reverse phase evaporation is an alternative method to the film hydration for preparing liposomal vesicles that involves formation of a water-in-oil emulsion between an aqueous phase and an organic phase containing lipids. A brief sonication of this mixture is required for system homogenization. The removal of the organic phase under reduced pressure yields a milky gel that turns subsequently into a liposomal suspension.

Other methods having organic solvent free characteristics may also be used according to the present disclosure for preparing a colloid.

LNPs typically consist of four components: ionizable cationic lipids, phospholipids, cholesterol, and polyethylene glycol (PEG)-lipids. Each component is responsible for payload protection, and enables effective intracellular delivery. LNPs may be prepared by mixing lipids dissolved in ethanol rapidly with nucleic acid in an aqueous buffer.

The term “average diameter” refers to the mean hydrodynamic diameter of particles as measured by dynamic laser light scattering (DLS) with data analysis using the so-called cumulant algorithm, which provides as results the so-called Z_{average} with the dimension of a length, and the polydispersity index (PI), which is dimensionless (Koppel, D., J. Chem. Phys. 57, 1972, pp 4814-4820, ISO 13321). Here “average diameter”, “diameter” or “size” for particles is used synonymously with this value of the Z_{average}.

The “polydispersity index” is preferably calculated based on dynamic light scattering measurements by the so-called cumulant analysis as mentioned in the definition of the “average diameter”, Under certain prerequisites, it can be taken as a measure of the size distribution of an ensemble of nanoparticles.

Different types of nucleic acid containing particles have been described previously to be suitable for delivery of nucleic acid in particulate form (e.g. Kaczmarek, J. C. et al., 2017, Genome Medicine 9, 60). For non-viral nucleic acid delivery vehicles, nanoparticle encapsulation of nucleic acid physically protects nucleic acid from degradation and, depending on the specific chemistry, can aid in cellular uptake and endosomal escape.

The present disclosure describes particles comprising nucleic acid, at least one cationic or cationically ionizable lipid or lipid-like material, and/or at least one cationic polymer which associate with nucleic acid to form nucleic acid particles and compositions comprising such particles. The nucleic acid particles may comprise nucleic acid which is complexed in different forms by non-covalent interactions to the particle. The particles described herein are not viral particles, in particular infectious viral particles, i.e., they are not able to virally infect cells.

Suitable cationic or cationically ionizable lipids or lipid-like materials and cationic polymers are those that form nucleic acid particles and are included by the term “particle forming components” or “particle forming agents”. The term “particle forming components” or “particle forming agents” relates to any components which associate with nucleic acid to form nucleic acid particles. Such components include any component which can be part of nucleic acid particles.

### Cationic Polymer

Given their high degree of chemical flexibility, polymers are commonly used materials for nanoparticle-based delivery. Typically, cationic polymers are used to electrostatically condense the negatively charged nucleic acid into nanoparticles. These positively charged groups often consist of amines that change their state of protonation in the pH range between 5.5 and 7.5, thought to lead to an ion imbalance that results in endosomal rupture. Polymers such as poly-L-lysine, polyamidoamine, protamine and polyethyleneimine, as well as naturally occurring polymers such as chitosan have all been applied to nucleic acid delivery and are suitable as cationic polymers herein. In addition, some investigators have synthesized polymers specifically for nucleic acid delivery. Poly(β-amino esters), in particular, have gained widespread use in nucleic acid delivery owing to their ease of synthesis and biodegradability. Such synthetic polymers are also suitable as cationic polymers herein.

A “polymer,” as used herein, is given its ordinary meaning, i.e., a molecular structure comprising one or more repeat units (monomers), connected by covalent bonds. The repeat units can all be identical, or in some cases, there can be more than one type of repeat unit present within the polymer. In some cases, the polymer is biologically derived, i.e., a biopolymer such as a protein. In some cases, additional moieties can also be present in the polymer, for example targeting moieties such as those described herein.

If more than one type of repeat unit is present within the polymer, then the polymer is said to be a “copolymer.” It is to be understood that the polymer being employed herein can be a copolymer. The repeat units forming the copolymer can be arranged in any fashion. For example, the repeat units can be arranged in a random order, in an alternating order, or as a “block” copolymer, i.e., comprising one or more regions each comprising a first repeat unit (e.g., a first block), and one or more regions each comprising a second repeat unit (e.g., a second block), etc. Block copolymers can have two (a diblock copolymer), three (a triblock copolymer), or more numbers of distinct blocks.

In certain embodiments, the polymer is biocompatible. Biocompatible polymers are polymers that typically do not result in significant cell death at moderate concentrations. In certain embodiments, the biocompatible polymer is biodegradable, i.e., the polymer is able to degrade, chemically and/or biologically, within a physiological environment, such as within the body.

In certain embodiments, polymer may be protamine or polyalkyleneimine, in particular protamine.

The term “protamine” refers to any of various strongly basic proteins of relatively low molecular weight that are rich in arginine and are found associated especially with DNA in place of somatic histones in the sperm cells of various animals (as fish). In particular, the term “protamine” refers to proteins found in fish sperm that are strongly basic, are soluble in water, are not coagulated by heat, and yield chiefly arginine upon hydrolysis. In purified form, they are used in a long-acting formulation of insulin and to neutralize the anticoagulant effects of heparin.

According to the disclosure, the term “protamine” as used herein is meant to comprise any protamine amino acid sequence obtained or derived from natural or biological sources including fragments thereof and multimeric forms of said amino acid sequence or fragment thereof as well as (synthesized) polypeptides which are artificial and specifically designed for specific purposes and cannot be isolated from native or biological sources.

In one embodiment, the polyalkyleneimine comprises polyethylenimine and/or polypropylenimine, preferably polyethyleneimine. A preferred polyalkyleneimine is polyethyleneimine (PEI). The average molecular weight of PEI is preferably 0.75·10² to 10⁷ Da, preferably 1000 to 10⁵ Da, more preferably 10000 to 40000 Da, more preferably 15000 to 30000 Da, even more preferably 20000 to 25000 Da.

Preferred according to the disclosure is linear polyalkyleneimine such as linear polyethyleneimine (PEI). Cationic polymers (including polycationic polymers) contemplated for use herein include any cationic polymers which are able to electrostatically bind nucleic acid. In one embodiment, cationic polymers contemplated for use herein include any cationic polymers with which nucleic acid can be associated, e.g. by forming complexes with the nucleic acid or forming vesicles in which the nucleic acid is enclosed or encapsulated.

Particles described herein may also comprise polymers other than cationic polymers, i.e., non-cationic polymers and/or anionic polymers. Collectively, anionic and neutral polymers are referred to herein as non-cationic polymers.

### Lipid and Lipid-Like Material

The terms “lipid” and “lipid-like material” are broadly defined herein as molecules which comprise one or more hydrophobic moieties or groups and optionally also one or more hydrophilic moieties or groups. Molecules comprising hydrophobic moieties and hydrophilic moieties are also frequently denoted as amphiphiles. Lipids are usually poorly soluble in water. In an aqueous environment, the amphiphilic nature allows the molecules to self-assemble into organized structures and different phases. One of those phases consists of lipid bilayers, as they are present in vesicles, multilamellar/unilamellar liposomes, or membranes in an aqueous environment. Hydrophobicity can be conferred by the inclusion of apolar groups that include, but are not limited to, long-chain saturated and unsaturated aliphatic hydrocarbon groups and such groups substituted by one or more aromatic, cycloaliphatic, or heterocyclic group(s). The hydrophilic groups may comprise polar and/or charged groups and include carbohydrates, phosphate, carboxylic, sulfate, amino, sulfhydryl, nitro, hydroxyl, and other like groups.

As used herein, the term “amphiphilic” refers to a molecule having both a polar portion and a non-polar portion. Often, an amphiphilic compound has a polar head attached to a long hydrophobic tail. In some embodiments, the polar portion is soluble in water, while the non-polar portion is insoluble in water. In addition, the polar portion may have either a formal positive charge, or a formal negative charge.

Alternatively, the polar portion may have both a formal positive and a negative charge, and be a zwitterion or inner salt. For purposes of the disclosure, the amphiphilic compound can be, but is not limited to, one or a plurality of natural or non-natural lipids and lipid-like compounds.

The term “lipid-like material”, “lipid-like compound” or “lipid-like molecule” relates to substances that structurally and/or functionally relate to lipids but may not be considered as lipids in a strict sense. For example, the term includes compounds that are able to form amphiphilic layers as they are present in vesicles, multilamellar/unilamellar liposomes, or membranes in an aqueous environment and includes surfactants, or synthesized compounds with both hydrophilic and hydrophobic moieties. Generally speaking, the term refers to molecules, which comprise hydrophilic and hydrophobic moieties with different structural organization, which may or may not be similar to that of lipids. As used herein, the term “lipid” is to be construed to cover both lipids and lipid-like materials unless otherwise indicated herein or clearly contradicted by context.

Specific examples of amphiphilic compounds that may be included in an amphiphilic layer include, but are not limited to, phospholipids, aminolipids and sphingolipids.

In certain embodiments, the amphiphilic compound is a lipid. The term “lipid” refers to a group of organic compounds that are characterized by being insoluble in water, but soluble in many organic solvents. Generally, lipids may be divided into eight categories: fatty acids, glycerolipids, glycerophospholipids, sphingolipids, saccharolipids, polyketides (derived from condensation of ketoacyl subunits), sterol lipids and prenol lipids (derived from condensation of isoprene subunits). Although the term “lipid” is sometimes used as a synonym for fats, fats are a subgroup of lipids called triglycerides. Lipids also encompass molecules such as fatty acids and their derivatives (including tri-, di-, monoglycerides, and phospholipids), as well as sterol-containing metabolites such as cholesterol.

Fatty acids, or fatty acid residues are a diverse group of molecules made of a hydrocarbon chain that terminates with a carboxylic acid group; this arrangement confers the molecule with a polar, hydrophilic end, and a nonpolar, hydrophobic end that is insoluble in water. The carbon chain, typically between four and 24 carbons long, may be saturated or unsaturated, and may be attached to functional groups containing oxygen, halogens, nitrogen, and sulfur. If a fatty acid contains a double bond, there is the possibility of either a cis or trans geometric isomerism, which significantly affects the molecule's configuration. Cis-double bonds cause the fatty acid chain to bend, an effect that is compounded with more double bonds in the chain. Other major lipid classes in the fatty acid category are the fatty esters and fatty amides.

Glycerolipids are composed of mono-, di-, and tri-substituted glycerols, the best-known being the fatty acid triesters of glycerol, called triglycerides. The word “triacylglycerol” is sometimes used synonymously with “triglyceride”. In these compounds, the three hydroxyl groups of glycerol are each esterified, typically by different fatty acids. Additional subclasses of glycerolipids are represented by glycosylglycerols, which are characterized by the presence of one or more sugar residues attached to glycerol via a glycosidic linkage.

The glycerophospholipids are amphipathic molecules (containing both hydrophobic and hydrophilic regions) that contain a glycerol core linked to two fatty acid-derived “tails” by ester linkages and to one “head” group by a phosphate ester linkage. Examples of glycerophospholipids, usually referred to as phospholipids (though sphingomyelins are also classified as phospholipids) are phosphatidylcholine (also known as PC, GPCho or lecithin), phosphatidylethanolamine (PE or GPEtn) and phosphatidylserine (PS or GPSer).

Sphingolipids are a complex family of compounds that share a common structural feature, a sphingoid base backbone. The major sphingoid base in mammals is commonly referred to as sphingosine. Ceramides (N-acyl-sphingoid bases) are a major subclass of sphingoid base derivatives with an amide-linked fatty acid. The fatty acids are typically saturated or mono-unsaturated with chain lengths from 16 to 26 carbon atoms. The major phosphosphingolipids of mammals are sphingomyelins (ceramide phosphocholines), whereas insects contain mainly ceramide phosphoethanolamines and fungi have phytoceramide phosphoinositols and mannose-containing headgroups. The glycosphingolipids are a diverse family of molecules composed of one or more sugar residues linked via a glycosidic bond to the sphingoid base. Examples of these are the simple and complex glycosphingolipids such as cerebrosides and gangliosides.

Sterol lipids, such as cholesterol and its derivatives, or tocopherol and its derivatives, are an important component of membrane lipids, along with the glycerophospholipids and sphingomyelins.

Saccharolipids describe compounds in which fatty acids are linked directly to a sugar backbone, forming structures that are compatible with membrane bilayers. In the saccharolipids, a monosaccharide substitutes for the glycerol backbone present in glycerolipids and glycerophospholipids. The most familiar saccharolipids are the acylated glucosamine precursors of the Lipid A component of the lipopolysaccharides in Gram-negative bacteria. Typical lipid A molecules are disaccharides of glucosamine, which are derivatized with as many as seven fatty-acyl chains. The minimal lipopolysaccharide required for growth in E. coli is Kdo2-Lipid A, a hexa-acylated disaccharide of glucosamine that is glycosylated with two 3-deoxy-D-manno-octulosonic acid (Kdo) residues.

Polyketides are synthesized by polymerization of acetyl and propionyl subunits by classic enzymes as well as iterative and multimodular enzymes that share mechanistic features with the fatty acid synthases. They comprise a large number of secondary metabolites and natural products from animal, plant, bacterial, fungal and marine sources, and have great structural diversity. Many polyketides are cyclic molecules whose backbones are often further modified by glycosylation, methylation, hydroxylation, oxidation, or other processes.

According to the disclosure, lipids and lipid-like materials may be cationic, anionic or neutral. Neutral lipids or lipid-like materials exist in an uncharged or neutral zwitterionic form at a selected pH.

### Cationic or Cationically Ionizable Lipids or Lipid-Like Materials

The nucleic acid particles described herein comprise at least one cationic or cationically ionizable lipid or lipid-like material as particle forming agent. Cationic or cationically ionizable lipids or lipid-like materials contemplated for use herein include any cationic or cationically ionizable lipids or lipid-like materials which are able to electrostatically bind nucleic acid. In one embodiment, cationic or cationically ionizable lipids or lipid-like materials contemplated for use herein can be associated with nucleic acid, e.g. by forming complexes with the nucleic acid or forming vesicles in which the nucleic acid is enclosed or encapsulated.

As used herein, a “cationic lipid” or “cationic lipid-like material” refers to a lipid or lipid-like material having a net positive charge. Cationic lipids or lipid-like materials bind negatively charged nucleic acid by electrostatic interaction. Generally, cationic lipids possess a lipophilic moiety, such as a sterol, an acyl chain, a diacyl or more acyl chains, and the head group of the lipid typically carries the positive charge.

In certain embodiments, a cationic lipid or lipid-like material has a net positive charge only at certain pH, in particular acidic pH, while it has preferably no net positive charge, preferably has no charge, i.e., it is neutral, at a different, preferably higher pH such as physiological pH. This ionizable behavior is thought to enhance efficacy through helping with endosomal escape and reducing toxicity as compared with particles that remain cationic at physiological pH.

For purposes of the present disclosure, such “cationically ionizable” lipids or lipid-like materials are comprised by the term “cationic lipid or lipid-like material” unless contradicted by the circumstances.

In one embodiment, the cationic or cationically ionizable lipid or lipid-like material comprises a head group which includes at least one nitrogen atom (N) which is positive charged or capable of being protonated. Examples of cationic lipids include, but are not limited to 1,2-dioleoyl-3-trimethylammonium propane (DOTAP); N,N-dimethyl-2,3-dioleyloxypropylamine (DODMA), 1,2-di-O-octadecenyl-3-trimethylammonium propane (DOTMA), 3-(N—(N′,N′-dimethylaminoethane)-carbamoyl)cholesterol (DC-Chol), dimethyldioctadecylammonium (DDAB); 1,2-dioleoyl-3-dimethylammonium-propane (DODAP); 1,2-diacyloxy-3-dimethylammonium propanes; 1,2-dialkyloxy-3-dimethylammonium propanes; dioctadecyldimethyl ammonium chloride (DODAC), 1,2-distearyloxy-N,N-dimethyl-3-aminopropane (DSDMA), 2,3-di(tetradecoxy)propyl-(2-hydroxyethyl)-dimethylazanium (DMRIE), 1,2-dimyristoyl-sn-glycero-3-ethylphosphocholine (DMEPC), 1,2-dinnyristoyl-3-trimethylammonium propane (DMTAP), 1,2-dioleyloxypropyl-3-dimethyl-hydroxyethyl ammonium bromide (DORIE), and 2,3-dioleoyloxy-N-[2(spermine carboxamide)ethyl]-N,N-dimethyl-1-propanamium trifluoroacetate (DOSPA), 1,2-dilinoleyloxy-N,N-dimethylaminopropane (DLinDMA), 1,2-dilinolenyloxy-N,N-dimethylaminopropane (DLenDMA), dioctadecylamidoglycyl spermine (DOGS), 3-dimethylamino-2-(cholest-5-en-3-beta-oxybutan-4-oxy)-1-(cis,cis-9,12-oc-tadecadienoxy)propane (CLinDMA), 2-[5′-(cholest-5-en-3-beta-oxy)-3′-oxapentoxy)-3-dimethyl-1-(cis,cis-9′,12′-octadecadienoxy)propane (CpLinDMA), N,N-dimethyl-3,4-dioleyloxybenzylamine (DMOBA), 1,2-N,N′-dioleylcarbamyl-3-dimethylaminopropane (DOcarbDAP), 2,3-Dilinoleoyloxy-N,N-dimethylpropylamine (DLinDAP), 1,2-N, N′-Dilinoleylcarbamyl-3-dimethylaminopropane (DLincarbDAP), 1,2-Dilinoleoylcarbamyl-3-dimethylaminopropane (DLinCDAP), 2,2-dilinoleyl-4-dimethylaminomethyl-[1,3]-dioxolane (DLin-K-DMA), 2,2-dilinoleyl-4-dimethylaminoethyl-[1,3]-dioxolane (DLin-K-XTC2-DMA), 2,2-dilinoleyl-4-(2-dimethylaminoethyl)-[1,3]-dioxolane (DLin-KC2-DMA), heptatriaconta-6,9,28,31-tetraen-19-yl-4-(dimethylamino)butanoate (DLin-MC3-DMA), N-(2-Hydroxyethyl)-N,N-dimethyl-2,3-bis(tetradecyloxy)-1-propanaminium bromide (DMRIE), (±)-N-(3-aminopropyl)-N,N-dimethyl-2,3-bis(cis-9-tetradecenyloxy)-1-propanaminium bromide (GAP-DMORIE), (±)-N-(3-aminopropyl)-N,N-dimethyl-2,3-bis(dodecyloxy)-1-propananninium bromide (GAP-DLRIE), (±)-N-(3-aminopropyl)-N,N-dimethyl-2,3-bis(tetradecyloxy)-1-propanaminium bromide (GAP-DMRIE), N-(2-Aminoethyl)-N,N-dimethyl-2,3-bis(tetradecyloxy)-1-propanaminium bromide (βAE-DMRIE), N-(4-carboxybenzyl)-N,N-dimethyl-2,3-bis(oleoyloxy)propan-1-aminium (DOBAQ), 2-({8-[(3β)-cholest-5-en-3-yloxy]octyl}oxy)-N,N-dimethyl-3-[(9Z,12Z)-octadeca-9,12-dien-1-yloxy]propan-1-amine (Octyl-CLinDMA), 1,2-dimyristoyl-3-dimethylammonium-propane (DMDAP), 1,2-dipalmitoyl-3-dimethylammonium-propane (DPDAP), N1-[2-((1S)-1-[(3-aminopropyl)amino]-4-[di(3-amino-propyl)amino]butylcarboxamido)ethyl]-3,4-di[oleyloxy]-benzamide (MVL5), 1,2-dioleoyl-sn-glycero-3-ethylphosphocholine (DOEPC), 2,3-bis(dodecyloxy)-N-(2-hydroxyethyl)-N,N-dimethylpropan-1-ammonium bromide (DLRIE), N-(2-aminoethyl)-N,N-dimethyl-2,3-bis(tetradecyloxy)propan-1-aminium bromide (DMORIE), di((Z)-non-2-en-1-yl) 8,8′-((((2(dimethylamino)ethyl)thio)carbonyl)azanediyl)dioctanoate (ATX), N,N-dimethyl-2,3-bis(dodecyloxy)propan-1-amine (DLDMA), N,N-dimethyl-2,3-bis(tetradecyloxy)propan-1-amine (DMDMA), Di((Z)-non-2-en-1-yl)-9-((4-(dimethylaminobutanoyl)oxy)heptadecanedioate (L319), N-Dodecyl-3-((2-dodecylcarbamoyl-ethyl)-{2-[(2-dodecylcarbamoyl-ethyl)-2-{(2-dodecylcarbamoyl-ethyl)-[2-(2-dodecylcarbamoyl-ethylamino)-ethyl]-aminoyethylamino)propionamide (lipidoid 98N₁₂-5), 1-[2-[bis(2-hydroxydodecyl)amino]ethyl-[2-[4-[2-[bis(2 hydroxydodecyl)amino]ethyl]piperazin-1-yl]ethyl]amino]dodecan-2-ol (lipidoid C12-200). Preferred are DOTAP, DODMA, DOTMA, DODAC, and DOSPA. In specific embodiments, the at least one cationic lipid is DOTAP.

In some embodiments, the cationic lipid may comprise from about 10 mol % to about 100 mol %, about 20 mol % to about 100 mol %, about 30 mol % to about 100 mol %, about 40 mol % to about 100 mol %, or about 50 mol % to about 100 mol % of the total lipid present in the particle.

### Additional Lipids or Lipid-Like Materials

Particles described herein may also comprise lipids or lipid-like materials other than cationic or cationically ionizable lipids or lipid-like materials, i.e., non-cationic lipids or lipid-like materials (including non-cationically ionizable lipids or lipid-like materials). Collectively, anionic and neutral lipids or lipid-like materials are referred to herein as non-cationic lipids or lipid-like materials. Optimizing the formulation of nucleic acid particles by addition of other hydrophobic moieties, such as cholesterol and lipids, in addition to an ionizable/cationic lipid or lipid-like material may enhance particle stability and efficacy of nucleic acid delivery.

An additional lipid or lipid-like material may be incorporated which may or may not affect the overall charge of the nucleic acid particles. In certain embodiments, the additional lipid or lipid-like material is a non-cationic lipid or lipid-like material. The non-cationic lipid may comprise, e.g., one or more anionic lipids and/or neutral lipids. As used herein, a “neutral lipid” refers to any of a number of lipid species that exist either in an uncharged or neutral zwitterionic form at a selected pH. In preferred embodiments, the additional lipid comprises one of the following neutral lipid components: (1) a phospholipid, (2) cholesterol or a derivative thereof; or (3) a mixture of a phospholipid and cholesterol or a derivative thereof. Examples of cholesterol derivatives include, but are not limited to, cholestanol, cholestanone, cholestenone, coprostanol, cholesteryl-2′-hydroxyethyl ether, cholesteryl-4′-hydroxybutyl ether, tocopherol and derivatives thereof, and mixtures thereof.

Specific phospholipids that can be used include, but are not limited to, phosphatidylcholines, phosphatidylethanolamines, phosphatidylglycerols, phosphatidic acids, phosphatidylserines or sphingomyelin. Such phospholipids include in particular diacylphosphatidylcholines, such as distearoylphosphatidylcholine (DSPC), dioleoylphosphatidylcholine (DOPC), dimyristoylphosphatidylcholine (DMPC), dipentadecanoylphosphatidylcholine, dilauroylphosphatidylcholine, dipalmitoylphosphatidylcholine (DPPC), diarachidoylphosphatidylcholine (DAPC), dibehenoylphosphatidylcholine (DBPC), ditricosanoylphosphatidylcholine (DTPC), dilignoceroylphatidylcholine (DLPC), palmitoyloleoyl-phosphatidylcholine (POPC), 1,2-di-O-octadecenyl-sn-glycero-3-phosphocholine (18:0 Diether PC), 1-oleoyl-2-cholesterylhemisuccinoyl-sn-glycero-3-phosphocholine (OChemsPC), 1-hexadecyl-sn-glycero-3-phosphocholine (C16 Lyso PC) and phosphatidylethanolamines, in particular diacylphosphatidylethanolamines, such as dioleoylphosphatidylethanolamine (DOPE), distearoyl-phosphatidylethanolamine (DSPE), dipalmitoyl-phosphatidylethanolamine (DPPE), dimyristoyl-phosphatidylethanolamine (DMPE), dilauroyl-phosphatidylethanolamine (DLPE), diphytanoyl-phosphatidylethanolamine (DPyPE), and further phosphatidylethanolamine lipids with different hydrophobic chains.

In certain preferred embodiments, the additional lipid is DSPC or DSPC and cholesterol.

In certain embodiments, the nucleic acid particles include both a cationic lipid and an additional lipid. In an exemplary embodiment, the cationic lipid is DOTAP and the additional lipid is DSPC or DSPC and cholesterol.

Without wishing to be bound by theory, the amount of the at least one cationic lipid compared to the amount of the at least one additional lipid may affect important nucleic acid particle characteristics, such as charge, particle size, stability, tissue selectivity, and bioactivity of the nucleic acid. Accordingly, in some embodiments, the molar ratio of the at least one cationic lipid to the at least one additional lipid is from about 10:0 to about 1:9, about 4:1 to about 1:2, or about 3:1 to about 1:1.

In some embodiments, the non-cationic lipid, in particular neutral lipid, (e.g., one or more phospholipids and/or cholesterol) may comprise from about 0 mol % to about 90 mol %, from about 0 mol % to about 80 mol %, from about 0 mol % to about 70 mol %, from about 0 mol % to about 60 mol %, or from about 0 mol % to about 50 mol %, of the total lipid present in the particle.

### Targeting Molecules

One or more of the particle-forming components described herein such as polymers, lipids and/or lipid-like materials may comprise or may be functionalized with one or more DARPins that will direct the particle to immune effector cells, in particular T cells such as CD8⁺ T cells. The DARPin may be conjugated, in particular covalently or non-covalently bound to or linked to, any particle forming component such as a lipid, lipid-like material or polymer.

Provided herein are, in particular, DARPins that when fused to nucleic acid particle components such as lipids or proteins specifically bind to CD8 exhibiting increased transfection of CD8+ T cells in vitro and in vivo as compared to non-DARPin-functionalized particles.

CD8 is a primary marker of the cytotoxic subset of T lymphocytes. CD8 is a type-I single pass transmembrane protein expressed as disulfide-linked homo- or heterodimeric molecule on the surface of immune cells. The CD8 heterodimer consists of the CD8a and CD813 chain and is only expressed on the surface of immature CD4+CD8+ double-positive thymocytes and mature peripheral cytotoxic αβ T cells.

The homodimer consists of two CD8a chains and exhibits expression on a much broader range of immune cells. In addition to classic cytotoxic αβ T cells and thymocytes, it is found on natural killer T (NKT) cells, a subset of dendritic cells (DC), and natural killer (NK) cell subpopulations. Both CD8αβ and CD8αα can mediate MHC-I binding; still, the heterodimeric form is more prevalent on the surface of MHC-I-restricted cytotoxic T cells. Of note, CD8ββ-homodimers do not occur naturally.

The term “DARPin” refers to designed ankyrin repeat proteins. DARPins are based on naturally occurring ankyrin repeat proteins, yet contain one or more amino acid mutations that can affect, for example, their binding affinity to a target molecule, their cell surface expression, and the like. DARPins preferably include 2 to 3 ankyrin repeat modules flanked by N- and C-capping repeats. Each ankyrin repeat module includes about 33 amino acid residues.

Ankyrin repeat proteins have been identified in 1987 through sequence comparisons between four such proteins in Saccharomyces cerevisiae, Drosophila melanogaster and Caenorhabditis elegans. Breeden and Nasmyth reported multiple copies of a repeat unit of approximately 33 residues in the sequences of swi6p, cddOp, notch and lin-12 (Breeden et al., Nature 329, 651-654 (1987)). The subsequent discovery of 24 copies of this repeat unit in the ankyrin protein led to the naming of this repeat unit as the ankyrin repeat (Lux et al., Nature 344, 36-42 (1990)). Later, this repeat unit has been identified in several hundreds of proteins of different organisms and viruses (Bork, Proteins 17(4), 363-74 (1993)). These proteins are located in the nucleus, the cytoplasm or the extracellular space. This is consistent with the fact that the ankyrin repeat domain of these proteins is independent of disulfide bridges and thus independent of the oxidation state of the environment. The number of repeat units per protein varies from two to more than twenty. Tertiary structures of ankyrin repeat units share a characteristic fold (Sedgwick and Smerdon, Trends Biochem Sci. 24(8), 311-6 (1999)) composed of a β-hairpin followed by two antiparallel α-helices and ending with a loop connecting the repeat unit with the next one. Domains built of ankyrin repeat units are formed by stacking the repeat units to an extended and curved structure. Proteins containing ankyrin repeat domains often contain additional domains. While the latter domains have variable functions, the function of the ankyrin repeat domain is most often the binding of other proteins. When analysing the repeat units of these proteins, the target interaction residues are mainly found in the β-hairpin and the exposed part of the first α-helix. These target interaction residues are hence forming a large contact surface on the ankyrin repeat domain. This contact surface is exposed on a framework built of stacked units of α-helix 1, α-helix 2 and the loop.

DARPins that bind to specific targets can be identified by screening combinatorial libraries of DARPins and selecting those with desired binding properties for the target. Such screening methods are described in, e.g., Muench et al., Molecular Therapy, 16(4), 686-693, 2011. For example, ribosomal display or phage display methods can be used to select target-specific DARPins from diverse libraries.

The term “repeat protein” refers to a (poly)peptide/protein comprising one or more repeat domains. In one embodiment, a repeat protein comprises up to four repeat domains. In one embodiment, a repeat protein comprises up to three repeat domains. In one embodiment, a repeat protein comprises up to two repeat domains. In the most preferred embodiment, a repeat protein comprises one repeat domain.

A repeat protein may comprise additional non-repeat protein domains such as (poly)peptide tags, enzymes (for example alkaline phosphatase) which may allow the detection of repeat proteins, or moieties which can be used for targeting (such as immunoglobulins or fragments thereof) and/or as effector molecules.

The term “(poly)peptide tag” refers to an amino acid sequence attached to a (poly)peptide/protein, where said amino acid sequence is useful for the purification, detection, or targeting of said (poly)peptide/protein. Such (poly)peptide tags may be small polypeptide sequences, for example, Hisₙ, myc, FLAG, or Strep-tag. These (poly)peptide tags are all well known in the art.

The individual domains of a repeat protein may be connected to each other directly or via (poly)peptide linkers. The term “(poly)peptide linker” refers to an amino acid sequence which is able to link two protein domains. Such linkers include, for example, glycine-serine-linkers of variable lengths and are known to the person skilled in the relevant art.

The term “repeat domain” refers to a protein domain comprising two or more consecutive repeat units (modules). In one embodiment, said repeat units are structural units having the same or a similar folding structure, and preferably stack tightly to preferably create a superhelical structure having a joint hydrophobic core.

The term “structural unit” refers to a locally ordered part of a (poly)peptide, formed by three-dimensional interactions between two or more segments of secondary structure that are near one another along the (poly)peptide chain. Such a structural unit comprises a structural motif.

The term “structural motif” refers to a three-dimensional arrangement of secondary structure elements present in at least one structural unit. Structural motifs are well known to the person skilled in the relevant art. Said structural units may alone not be able to acquire a defined three-dimensional arrangement; however, their consecutive arrangement as repeat modules in a repeat domain leads to a mutual stabilization of neighbouring units which may result in a superhelical structure.

The term “repeat modules” refers to the repeated amino acid sequences of the repeat proteins, which are derived from the repeat units of naturally occurring proteins. Each repeat module comprised in a repeat domain is derived from one or more repeat units of a family of naturally occurring repeat proteins, e.g., ankyrin repeat proteins.

The term “set of repeat modules” refers to the total number of repeat modules present in a repeat domain. Such “set of repeat modules” present in a repeat domain comprises two or more consecutive repeat modules, and may comprise just one type of repeat module in two or more copies, or two or more different types of modules, each present in one or more copies. Such set of repeat modules comprising, for example, 3 repeat modules may comprise consecutively, form N- to C-terminus, repeat module 1, repeat module 2, and repeat module 3, as shown for example, in FIG. 5. Repeat module 1 as shown in FIG. 5 preferably comprises amino acids 29 to 61. Repeat module 2 as shown in FIG. 5 preferably comprises amino acids 62 to 94. Repeat module 3 as shown in FIG. 5 preferably comprises amino acids 95 to 127.

Different repeat domains may have an identical number of repeat modules per repeat domain or may differ in the number of repeat modules per repeat domain.

Preferably, the repeat modules comprised in a set are homologous repeat modules. In the context of the present invention, the term “homologous repeat modules” refers to repeat modules, wherein more than 70% of the framework residues of said repeat modules are homologous. Preferably, more than 80% of the framework residues of said repeat modules are homologous. Most preferably, more than 90% of the framework residues of said repeat modules are homologous. Computer programs to determine the percentage of homology between polypeptides, such as Fasta, Blast or Gap, are known to the person skilled in the relevant art.

The term “repeat unit” refers to amino acid sequences comprising sequence motifs of one or more naturally occurring proteins, wherein said “repeat units” are found in multiple copies, and which exhibit a defined folding topology common to all said motifs determining the fold of the protein. Such repeat units comprise framework residues and interaction residues.

One example of such repeat units is an ankyrin repeat unit. Naturally occurring proteins containing two or more such repeat units are referred to as “naturally occurring repeat proteins”. The amino acid sequences of the individual repeat units of a repeat protein may have a significant number of mutations, substitutions, additions and/or deletions when compared to each other, while still substantially retaining the general pattern, or motif, of the repeat units.

The term “repeat sequence motif” or “repeat consensus sequence” refers to an amino acid sequence, which is deduced from one or more repeat units. Such repeat sequence motifs comprise framework residue positions and target interaction residue positions. Said framework residue positions correspond to the positions of framework residues of said repeat units. Said target interaction residue positions correspond to the positions of target interaction residues of said repeat units. Such repeat sequence motifs comprise fixed positions and randomized positions. The term “fixed position” refers to an amino acid position in a repeat sequence motif, wherein said position is set to a particular amino acid. Frequently, such fixed positions correspond to the positions of framework residues.

The term “randomized position” refers to an amino acid position in a repeat sequence motif, wherein two or more amino acids are allowed at said amino acid position. Frequently, such randomized positions correspond to the positions of target target interaction residues. However, some positions of framework residues may also be randomized.

The term “folding topology” refers to the tertiary structure of said repeat units. The folding topology will be determined by stretches of amino acids forming at least parts of α-helices or β-sheets, or amino acid stretches forming linear polypeptides or loops, or any combination of α-helices, β-sheets and/or linear polypeptides/loops.

The term “consecutive” refers to an arrangement, wherein said modules are arranged in tandem.

In repeat proteins, there are at least 2, frequently 6 or more, 10 or more, or 20 or more repeat units, usually about 2 to 6 repeat units. For the most part, the repeat proteins are structural proteins and/or adhesive proteins, being present in prokaryotes and eukaryotes, including vertebrates and non-vertebrates.

In most cases, said repeat units will exhibit a high degree of sequence identity (same amino acid residues at corresponding positions) or sequence similarity (amino acid residues being different, but having similar physicochemical properties), and some of the amino acid residues might be key residues being strongly conserved in the different repeat units found in naturally occurring proteins.

However, a high degree of sequence variability by amino acid insertions and/or deletions, and/or substitutions between the different repeat units found in naturally occurring proteins will be possible as long as the common folding topology is maintained.

The term “framework residues” relates to amino acid residues of the repeat units, or the corresponding amino acid residues of the repeat modules, which contribute to the folding topology, i.e. which contribute to the fold of said repeat unit (or module) or which contribute to the interaction with a neighboring unit (or module). Such contribution might be the interaction with other residues in the repeat unit (module), or the influence on the polypeptide backbone conformation as found in α-helices or β-sheets, or amino acid stretches forming linear polypeptides or loops.

The term “target interaction residues” refers to amino acid residues of the repeat units, or the corresponding amino acid residues of the repeat modules, which contribute to the interaction with target substances. Such contribution might be the direct interaction with the target substances, or the influence on other directly interacting residues, e.g. by stabilising the conformation of the (poly)peptide of said repeat unit (module) to allow or enhance the interaction of said directly interacting residues with said target.

A “target” may be an individual molecule such as a nucleic acid molecule, a (poly)peptide protein, a carbohydrate, or any other naturally occurring molecule, including any part of such individual molecule, or complexes of two or more of such molecules. The target may be, in particular, a molecule on immune effector cells, in particular CD8.

In one embodiment, the repeat modules are directly connected. In the context of the present invention, the term “directly connected” refers to repeat modules, which are arranged as direct repeats in a repeat protein without an intervening amino acid sequence.

In another embodiment, the repeat modules are connected by a (poly)peptide linker. Thus, the repeat modules may be linked indirectly via a (poly)peptide linker as intervening sequence separating the individual modules. An “intervening sequence” may be any amino acid sequence, which allows to connect the individual modules without interfering with the folding topology or the stacking of the modules. Preferentially, said intervening sequences are short (poly)peptide linkers of less than 10, and even more preferably, of less than 5 amino acid residues.

In one embodiment, a repeat protein further comprises an N- and/or a C-terminal capping module having an amino acid sequence different from any one of said repeat modules. The term “capping module” refers to a polypeptide fused to the N- or C-terminal repeat module of a repeat domain, wherein said capping module forms tight tertiary interactions with said repeat module thereby providing a cap that shields the hydrophobic core of said repeat module at the side not in contact with the consecutive repeat module from the solvent.

Said N- and/or C-terminal capping module may be, or may be derived from, a capping unit or other domain found in a naturally occurring repeat protein adjacent to a repeat unit.

The term “capping unit” refers to a naturally occurring folded (poly)peptide, wherein said (poly)peptide defines a particular structural unit which is N- or C-terminally fused to a repeat unit, wherein said (poly)peptide forms tight tertiary interactions with said repeat unit thereby providing a cap that shields the hydrophobic core of said repeat unit at one side from the solvent. Such capping units may have sequence similarities to said repeat sequence motif.

### Nucleic Acids

The term “polynucleotide” or “nucleic acid”, as used herein, is intended to include DNA and RNA such as genomic DNA, cDNA, mRNA, recombinantly produced and chemically synthesized molecules. A nucleic acid may be single-stranded or double-stranded. RNA includes in vitro transcribed RNA (IVT RNA) or synthetic RNA. According to the invention, a polynucleotide is preferably isolated.

Nucleic acids may be comprised in a vector. The term “vector” as used herein includes any vectors known to the skilled person including plasmid vectors, cosmid vectors, phage vectors such as lambda phage, viral vectors such as retroviral, adenoviral or baculoviral vectors, or artificial chromosome vectors such as bacterial artificial chromosomes (BAC), yeast artificial chromosomes (YAC), or P1 artificial chromosomes (PAC). Said vectors include expression as well as cloning vectors. Expression vectors comprise plasmids as well as viral vectors and generally contain a desired coding sequence and appropriate DNA sequences necessary for the expression of the operably linked coding sequence in a particular host organism (e.g., bacteria, yeast, plant, insect, or mammal) or in in vitro expression systems. Cloning vectors are generally used to engineer and amplify a certain desired DNA fragment and may lack functional sequences needed for expression of the desired DNA fragments.

In one embodiment of all aspects of the invention, nucleic acid such as nucleic acid encoding an antigen receptor or nucleic acid encoding a vaccine antigen is expressed in cells of the subject treated to provide the antigen receptor or vaccine antigen. In one embodiment of all aspects of the invention, the nucleic acid is transiently expressed in cells of the subject. Thus, in one embodiment, the nucleic acid is not integrated into the genome of the cells. In one embodiment of all aspects of the invention, the nucleic acid is RNA, preferably in vitro transcribed RNA. In one embodiment of all aspects of the invention, expression of the antigen receptor is at the cell surface. In one embodiment of all aspects of the invention, expression of the vaccine antigen is at the cell surface. In one embodiment of all aspects of the invention, the vaccine antigen is expressed and presented in the context of MHC.

In one embodiment of all aspects of the invention, the nucleic acid encoding the vaccine antigen is expressed in cells such as antigen presenting cells of the subject treated to provide the vaccine antigen for binding by the immune effector cells genetically modified to express an antigen receptor, said binding resulting in stimulation, priming and/or expansion of the immune effector cells genetically modified to express an antigen receptor.

The nucleic acids described herein may be recombinant and/or isolated molecules.

In the present disclosure, the term “RNA” relates to a nucleic acid molecule which includes ribonucleotide residues. In preferred embodiments, the RNA contains all or a majority of ribonucleotide residues. As used herein, “ribonucleotide” refers to a nucleotide with a hydroxyl group at the 2′-position of a 13-D-ribofuranosyl group. RNA encompasses without limitation, double stranded RNA, single stranded RNA, isolated RNA such as partially purified RNA, essentially pure RNA, synthetic RNA, recombinantly produced RNA, as well as modified RNA that differs from naturally occurring RNA by the addition, deletion, substitution and/or alteration of one or more nucleotides. Such alterations may refer to addition of non-nucleotide material to internal RNA nucleotides or to the end(s) of RNA. It is also contemplated herein that nucleotides in RNA may be non-standard nucleotides, such as chemically synthesized nucleotides or deoxynucleotides. For the present disclosure, these altered RNAs are considered analogs of naturally-occurring RNA.

In certain embodiments of the present disclosure, the RNA is messenger RNA (mRNA) that relates to a RNA transcript which encodes a peptide or protein. As established in the art, mRNA generally contains a 5′ untranslated region (5′-UTR), a peptide coding region and a 3′ untranslated region (3′-UTR). In some embodiments, the RNA is produced by in vitro transcription or chemical synthesis. In one embodiment, the mRNA is produced by in vitro transcription using a DNA template where DNA refers to a nucleic acid that contains deoxyribonucleotides.

In one embodiment, RNA is in vitro transcribed RNA (IVT-RNA) and may be obtained by in vitro transcription of an appropriate DNA template. The promoter for controlling transcription can be any promoter for any RNA polymerase. A DNA template for in vitro transcription may be obtained by cloning of a nucleic acid, in particular cDNA, and introducing it into an appropriate vector for in vitro transcription. The cDNA may be obtained by reverse transcription of RNA.

In one embodiment, the RNA may have modified ribonucleotides. Examples of modified ribonucleotides include, without limitation, 5-methylcytidine, pseudouridine and/or 1-methyl-pseudouridine.

In some embodiments, the RNA according to the present disclosure comprises a 5′-cap. In one embodiment, the RNA of the present disclosure does not have uncapped 5′-triphosphates. In one embodiment, the RNA may be modified by a 5′-cap analog. The term “5′-cap” refers to a structure found on the 5′-end of an mRNA molecule and generally consists of a guanosine nucleotide connected to the mRNA via a 5′ to 5′ triphosphate linkage. In one embodiment, this guanosine is methylated at the 7-position. Providing an RNA with a 5′-cap or 5′-cap analog may be achieved by in vitro transcription, in which the 5′-cap is co-transcriptionally expressed into the RNA strand, or may be attached to RNA post-transcriptionally using capping enzymes.

In some embodiments, the building block cap for RNA is m₂^{7,3′-O}Gppp(m₁^{2′-O})ApG (also sometimes referred to as m₂^{7,3′O}G(5′)ppp(5′)m^{2′-O}ApG), which has the following structure:

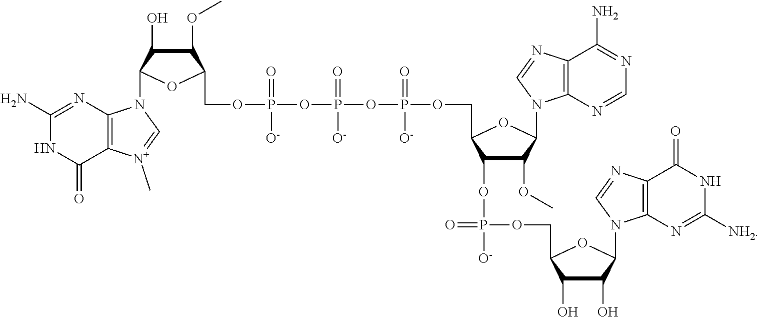

Below is an exemplary Cap1 RNA, which comprises RNA and m₂^{7,3′O}G(5′)ppp(5′)m^{2′-O}ApG:

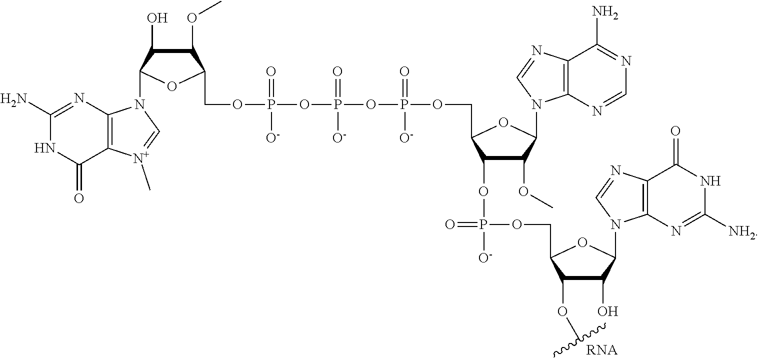

Below is another exemplary Cap1 RNA (no cap analog):

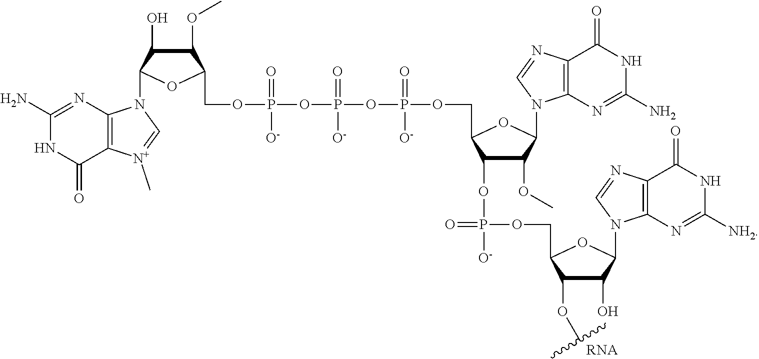

In some embodiments, the RNA is modified with “Cap0” structures using, in one embodiment, the cap analog anti-reverse cap (ARCA Cap (m₂^{7,3′O}G(5′)ppp(5′)G)) with the structure:

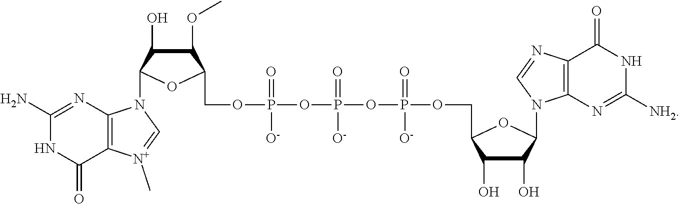

Below is an exemplary Cap0 RNA comprising RNA and m₂^{7,3′O}G(5′)ppp(5′)G:

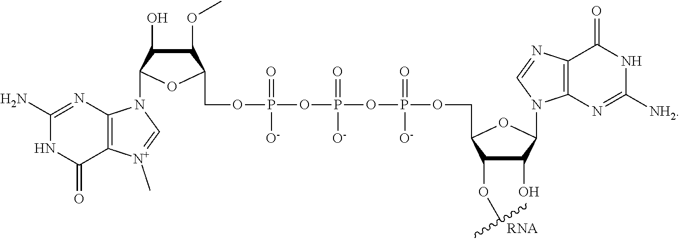

In some embodiments, the “Cap0” structures are generated using the cap analog Beta-S-ARCA (m₂^{7,2′O}G(5′)ppSp(5′)G) with the structure:

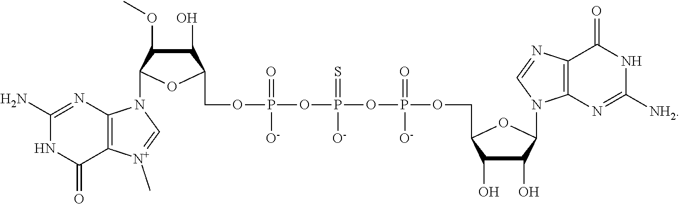

Below is an exemplary Cap0 RNA comprising Beta-S-ARCA (m₂^{7,2′O}G(5′)ppSp(5′)G) and RNA:

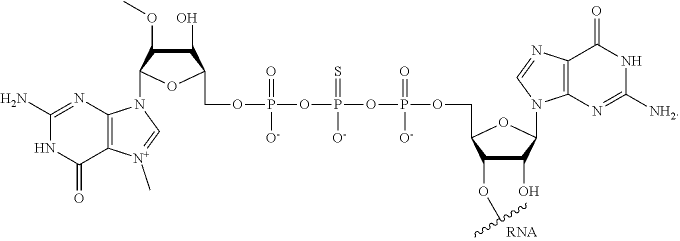

A particularly preferred Cap comprises the 5′-cap m₂^{7,2′O}G(5′)ppSp(5′)G.

In some embodiments, RNA according to the present disclosure comprises a 5′-UTR and/or a 3′-UTR. The term “untranslated region” or “UTR” relates to a region in a DNA molecule which is transcribed but is not translated into an amino acid sequence, or to the corresponding region in an RNA molecule, such as an mRNA molecule. An untranslated region (UTR) can be present 5′ (upstream) of an open reading frame (5′-UTR) and/or 3′ (downstream) of an open reading frame (3′-UTR). A 5′-UTR, if present, is located at the 5′ end, upstream of the start codon of a protein-encoding region. A 5′-UTR is downstream of the 5′-cap (if present), e.g. directly adjacent to the 5′-cap. A 3′-UTR, if present, is located at the 3′ end, downstream of the termination codon of a protein-encoding region, but the term “3′-UTR” does preferably not include the poly(A) tail. Thus, the 3′-UTR is upstream of the poly(A) sequence (if present), e.g. directly adjacent to the poly(A) sequence.

In some embodiments, the RNA according to the present disclosure comprises a 3′-poly(A) sequence. As used herein, the term “poly-A tail” or “poly-A sequence” refers to an uninterrupted or interrupted sequence of adenylate residues which is typically located at the 3′-end of an RNA molecule. Poly-A tails or poly-A sequences are known to those of skill in the art and may follow the 3′-UTR in the RNAs described herein. An uninterrupted poly-A tail is characterized by consecutive adenylate residues. In nature, an uninterrupted poly-A tail is typical. RNAs disclosed herein can have a poly-A tail attached to the free 3′-end of the RNA by a template-independent RNA polymerase after transcription or a poly-A tail encoded by DNA and transcribed by a template-dependent RNA polymerase. It has been demonstrated that a poly-A tail of about 120 A nucleotides has a beneficial influence on the levels of RNA in transfected eukaryotic cells, as well as on the levels of protein that is translated from an open reading frame that is present upstream (5′) of the poly-A tail (Holtkamp et al., 2006, Blood, vol. 108, pp. 4009-4017).

The poly-A tail may be of any length. In some embodiments, a poly-A tail comprises, essentially consists of, or consists of at least 20, at least 30, at least 40, at least 80, or at least 100 and up to 500, up to 400, up to 300, up to 200, or up to 150 A nucleotides, and, in particular, about 120 A nucleotides. In this context, “essentially consists of” means that most nucleotides in the poly-A tail, typically at least 75%, at least 80%, at least 85%, at least 90%, at least 95%, at least 96%, at least 97%, at least 98%, or at least 99% by number of nucleotides in the poly-A tail are A nucleotides, but permits that remaining nucleotides are nucleotides other than A nucleotides, such as U nucleotides (uridylate), G nucleotides (guanylate), or C nucleotides (cytidylate). In this context, “consists of” means that all nucleotides in the poly-A tail, i.e., 100% by number of nucleotides in the poly-A tail, are A nucleotides. The term “A nucleotide” or “A” refers to adenylate.

In some embodiments, a poly-A tail is attached during RNA transcription, e.g., during preparation of in vitro transcribed RNA, based on a DNA template comprising repeated dT nucleotides (deoxythymidylate) in the strand complementary to the coding strand. The DNA sequence encoding a poly-A tail (coding strand) is referred to as poly(A) cassette.

In some embodiments, the poly(A) cassette present in the coding strand of DNA essentially consists of dA nucleotides, but is interrupted by a random sequence of the four nucleotides (dA, dC, dG, and dT). Such random sequence may be 5 to 50, 10 to 30, or 10 to 20 nucleotides in length. Such a cassette is disclosed in WO 2016/005324 A1, hereby incorporated by reference. Any poly(A) cassette disclosed in WO 2016/005324 A1 may be used in the present invention. A poly(A) cassette that essentially consists of dA nucleotides, but is interrupted by a random sequence having an equal distribution of the four nucleotides (dA, dC, dG, dT) and having a length of e.g., 5 to 50 nucleotides shows, on DNA level, constant propagation of plasmid DNA in E. coli and is still associated, on RNA level, with the beneficial properties with respect to supporting RNA stability and translational efficiency is encompassed. Consequently, in some embodiments, the poly-A tail contained in an RNA molecule described herein essentially consists of A nucleotides, but is interrupted by a random sequence of the four nucleotides (A, C, G, U). Such random sequence may be 5 to 50, 10 to 30, or 10 to 20 nucleotides in length. In some embodiments, no nucleotides other than A nucleotides flank a poly-A tail at its 3′-end, i.e., the poly-A tail is not masked or followed at its 3′-end by a nucleotide other than A.

In some embodiments, the poly-A tail may comprise at least 20, at least 30, at least 40, at least 80, or at least 100 and up to 500, up to 400, up to 300, up to 200, or up to 150 nucleotides. In some embodiments, the poly-A tail may essentially consist of at least 20, at least 30, at least 40, at least 80, or at least 100 and up to 500, up to 400, up to 300, up to 200, or up to 150 nucleotides. In some embodiments, the poly-A tail may consist of at least 20, at least 30, at least 40, at least 80, or at least 100 and up to 500, up to 400, up to 300, up to 200, or up to 150 nucleotides. In some embodiments, the poly-A tail comprises at least 100 nucleotides. In some embodiments, the poly-A tail comprises about 150 nucleotides. In some embodiments, the poly-A tail comprises about 120 nucleotides.

According to the disclosure, vaccine antigen is preferably administered as single-stranded, 5′-capped mRNA that is translated into the respective protein upon entering antigen-presenting cells (APCs). Preferably, the RNA contains structural elements optimized for maximal efficacy of the RNA with respect to stability and translational efficiency (5′-cap, 5′-UTR, 3′-UTR, poly(A)-tail).

In one embodiment, beta-S-ARCA(D1) is utilized as specific capping structure at the 5′-end of the RNA. In one embodiment, the 5′-UTR sequence is derived from the human alpha-globin mRNA. In one embodiment, two re-iterated 3′-UTRs derived from the human beta-globin mRNA are placed between the coding sequence and the poly(A)-tail to assure higher maximum protein levels and prolonged persistence of the mRNA. In one embodiment, a poly(A)-tail measuring 110 nucleotides in length, consisting of a stretch of 30 adenosine residues, followed by a 10 nucleotide linker sequence and another 70 adenosine residues is used. This poly(A)-tail sequence was designed to enhance RNA stability and translational efficiency in dendritic cells.

The RNA is preferably administered as lipoplex particles, preferably comprising DOTMA and DOPE, as further described below. Such particles are preferably administered by systemic administration, in particular by intravenous administration.

In the context of the present disclosure, the term “transcription” relates to a process, wherein the genetic code in a DNA sequence is transcribed into RNA. Subsequently, the RNA may be translated into peptide or protein.

With respect to RNA, the term “expression” or “translation” relates to the process in the ribosomes of a cell by which a strand of mRNA directs the assembly of a sequence of amino acids to make a peptide or protein.

“Encoding” refers to the inherent property of specific sequences of nucleotides in a polynucleotide, such as a gene, a cDNA, or an mRNA, to serve as templates for synthesis of other polymers and macromolecules in biological processes having either a defined sequence of nucleotides (i.e., rRNA, tRNA and mRNA) or a defined sequence of amino acids and the biological properties resulting therefrom. Thus, a gene encodes a protein if transcription and translation of mRNA corresponding to that gene produces the protein in a cell or other biological system. Both the coding strand, the nucleotide sequence of which is identical to the mRNA sequence and is usually provided in sequence listings, and the non-coding strand, used as the template for transcription of a gene or cDNA, can be referred to as encoding the protein or other product of that gene or cDNA.

According to the disclosure, the term “RNA encodes” means that the RNA, if present in the appropriate environment, such as within cells of a target tissue, can direct the assembly of amino acids to produce the peptide or protein it encodes during the process of translation. In one embodiment, RNA is able to interact with the cellular translation machinery allowing translation of the peptide or protein. A cell may produce the encoded peptide or protein intracellularly (e.g. in the cytoplasm and/or in the nucleus), may secrete the encoded peptide or protein, or may produce it on the surface.

As used herein “endogenous” refers to any material from or produced inside an organism, cell, tissue or system.

As used herein, the term “exogenous” refers to any material introduced from or produced outside an organism, cell, tissue or system.

The term “expression” as used herein is defined as the transcription and/or translation of a particular nucleotide sequence. Expression can be transient or stable. According to the invention, the term expression also includes an “aberrant expression” or “abnormal expression”.

As used herein, the terms “linked,” “fused”, or “fusion” are used interchangeably. These terms refer to the joining together of two or more elements or components or domains.

### Cytokines

The methods described herein may comprise providing to a subject one or more cytokines, e.g., by administering to the subject the one or more cytokines, a polynucleotide encoding the one or more cytokines or a host cell expressing the one or more cytokines.

The term “cytokine” as used herein includes naturally occurring cytokines and functional variants thereof (including fragments of the naturally occurring cytokines and variants thereof). One particularly preferred cytokine is IL2.

Cytokines are a category of small proteins (˜5-20 kDa) that are important in cell signaling. Their release has an effect on the behavior of cells around them. Cytokines are involved in autocrine signaling, paracrine signaling and endocrine signaling as immunomodulating agents. Cytokines include chemokines, interferons, interleukins, lymphokines, and tumour necrosis factors but generally not hormones or growth factors (despite some overlap in the terminology). Cytokines are produced by a broad range of cells, including immune cells like macrophages, B lymphocytes, T lymphocytes and mast cells, as well as endothelial cells, fibroblasts, and various stromal cells. A given cytokine may be produced by more than one type of cell. Cytokines act through receptors, and are especially important in the immune system; cytokines modulate the balance between humoral and cell-based immune responses, and they regulate the maturation, growth, and responsiveness of particular cell populations. Some cytokines enhance or inhibit the action of other cytokines in complex ways.

### IL2

Interleukin-2 (IL2) is a cytokine that induces proliferation of antigen-activated T cells and stimulates natural killer (NK) cells. The biological activity of IL2 is mediated through a multi-subunit IL2 receptor complex (IL2R) of three polypeptide subunits that span the cell membrane: p55 (IL2Ra, the alpha subunit, also known as CD25 in humans), p75 (IL2R13, the beta subunit, also known as CD122 in humans) and p64 (IL2Rγ, the gamma subunit, also known as CD132 in humans). T cell response to IL2 depends on a variety of factors, including: (1) the concentration of IL2; (2) the number of IL2R molecules on the cell surface; and (3) the number of IL2R occupied by IL2 (i.e., the affinity of the binding interaction between 11_2 and IL2R (Smith, “Cell Growth Signal Transduction is Quantal” In Receptor Activation by Antigens, Cytokines, Hormones, and Growth Factors 766:263-271, 1995)). The IL2:IL2R complex is internalized upon ligand binding and the different components undergo differential sorting. When administered as an intravenous (i.v.) bolus, IL2 has a rapid systemic clearance (an initial clearance phase with a half-life of 12.9 minutes followed by a slower clearance phase with a half-life of 85 minutes) (Konrad et al., Cancer Res. 50:2009-2017, 1990).

In eukaryotic cells human IL2 is synthesized as a precursor polypeptide of 153 amino acids, from which 20 amino acids are removed to generate mature secreted IL2. Recombinant human IL2 has been produced in E. coli, in insect cells and in mammalian COS cells.

According to the disclosure, IL2 (optionally as a portion of extended-PK IL2) may be naturally occurring IL2 or a fragment or variant thereof. IL2 may be human IL2 and may be derived from any vertebrate, especially any mammal.

### Extended-PK Group

Cytokine polypeptides described herein can be prepared as fusion or chimeric polypeptides that include a cytokine portion and a heterologous polypeptide (i.e., a polypeptide that is not a cytokine or a variant thereof). The resulting molecule, hereafter referred to as “extended-pharmacokinetic (PK) cytokine,” has a prolonged circulation half-life relative to free cytokine. The prolonged circulation half-life of extended-PK cytokine permits in vivo serum cytokine concentrations to be maintained within a therapeutic range, potentially leading to the enhanced activation of many types of immune cells, including T cells. Because of its favorable pharmacokinetic profile, extended-PK cytokine can be dosed less frequently and for longer periods of time when compared with unmodified cytokine.

As used herein, “half-life” refers to the time taken for the serum or plasma concentration of a compound such as a peptide or protein to reduce by 50%, in vivo, for example due to degradation and/or clearance or sequestration by natural mechanisms. An extended-PK cytokine such as an extended-PK interleukin (IL) suitable for use herein is stabilized in vivo and its half-life increased by, e.g., fusion to serum albumin (e.g., HSA or MSA), which resist degradation and/or clearance or sequestration. The half-life can be determined in any manner known per se, such as by pharmacokinetic analysis. Suitable techniques will be clear to the person skilled in the art, and may for example generally involve the steps of suitably administering a suitable dose of the amino acid sequence or compound to a subject; collecting blood samples or other samples from said subject at regular intervals; determining the level or concentration of the amino acid sequence or compound in said blood sample; and calculating, from (a plot of) the data thus obtained, the time until the level or concentration of the amino acid sequence or compound has been reduced by 50% compared to the initial level upon dosing. Further details are provided in, e.g., standard handbooks, such as Kenneth, A. et al., Chemical Stability of Pharmaceuticals: A Handbook for Pharmacists and in Peters et al., Pharmacokinetic Analysis: A Practical Approach (1996). Reference is also made to Gibaldi, M. et al., Pharmacokinetics, 2nd Rev. Edition, Marcel Dekker (1982).

The cytokine may be fused to an extended-PK group, which increases circulation half-life. Non-limiting examples of extended-PK groups are described infra. It should be understood that other PK groups that increase the circulation half-life of cytokines, or variants thereof, are also applicable to the present disclosure. In certain embodiments, the extended-PK group is a serum albumin domain (e.g., mouse serum albumin, human serum albumin).

As used herein, the term “PK” is an acronym for “pharmacokinetic” and encompasses properties of a compound including, by way of example, absorption, distribution, metabolism, and elimination by a subject. As used herein, an “extended-PK group” refers to a protein, peptide, or moiety that increases the circulation half-life of a biologically active molecule when fused to or administered together with the biologically active molecule. Examples of an extended-PK group include serum albumin (e.g., HSA), Immunoglobulin Fc or Fc fragments and variants thereof, transferrin and variants thereof, and human serum albumin (HSA) binders (as disclosed in U.S. Publication Nos. 2005/0287153 and 2007/0003549). Other exemplary extended-PK groups are disclosed in Kontermann, Expert Opin Biol Ther, 2016 July; 16(7):903-15 which is herein incorporated by reference in its entirety. As used herein, an “extended-PK cytokine” refers to a cytokine moiety in combination with an extended-PK group. In one embodiment, the extended-PK cytokine is a fusion protein in which a cytokine moiety is linked or fused to an extended-PK group. As used herein, an “extended-PK IL” refers to an interleukin (IL) moiety (including an IL variant moiety) in combination with an extended-PK group. In one embodiment, the extended-PK IL is a fusion protein in which an IL moiety is linked or fused to an extended-PK group. An exemplary fusion protein is an HSA/IL2 fusion in which an IL2 moiety is fused with HSA.

In certain embodiments, the serum half-life of an extended-PK cytokine is increased relative to the cytokine alone (i.e., the cytokine not fused to an extended-PK group). In certain embodiments, the serum half-life of the extended-PK cytokine is at least 20, 40, 60, 80, 100, 120, 150, 180, 200, 400, 600, 800, or 1000% longer relative to the serum half-life of the cytokine alone. In certain embodiments, the serum half-life of the extended-PK cytokine is at least 1.5-fold, 2-fold, 2.5-fold, 3-fold, 3.5 fold, 4-fold, 4.5-fold, 5-fold, 6-fold, 7-fold, 8-fold, 10-fold, 12-fold, 13-fold, 15-fold, 17-fold, 20-fold, 22-fold, 25-fold, 27-fold, 30-fold, 35-fold, 40-fold, or 50-fold greater than the serum half-life of the cytokine alone. In certain embodiments, the serum half-life of the extended-PK cytokine is at least 10 hours, 15 hours, 20 hours, 25 hours, 30 hours, 35 hours, 40 hours, 50 hours, 60 hours, 70 hours, 80 hours, 90 hours, 100 hours, 110 hours, 120 hours, 130 hours, 135 hours, 140 hours, 150 hours, 160 hours, or 200 hours.

In certain embodiments, the extended-PK group includes serum albumin, or fragments thereof or variants of the serum albumin or fragments thereof (all of which for the purpose of the present disclosure are comprised by the term “albumin”). Polypeptides described herein may be fused to albumin (or a fragment or variant thereof) to form albumin fusion proteins. Such albumin fusion proteins are described in U.S. Publication No. 20070048282.

As used herein, “albumin fusion protein” refers to a protein formed by the fusion of at least one molecule of albumin (or a fragment or variant thereof) to at least one molecule of a protein such as a therapeutic protein, in particular IL2 (or variant thereof). The albumin fusion protein may be generated by translation of a nucleic acid in which a polynucleotide encoding a therapeutic protein is joined in-frame with a polynucleotide encoding an albumin. The therapeutic protein and albumin, once part of the albumin fusion protein, may each be referred to as a “portion”, “region” or “moiety” of the albumin fusion protein (e.g., a “therapeutic protein portion” or an “albumin protein portion”). In a highly preferred embodiment, an albumin fusion protein comprises at least one molecule of a therapeutic protein (including, but not limited to a mature form of the therapeutic protein) and at least one molecule of albumin (including but not limited to a mature form of albumin). In one embodiment, an albumin fusion protein is processed by a host cell such as a cell of the target organ for administered RNA, e.g. a liver cell, and secreted into the circulation. Processing of the nascent albumin fusion protein that occurs in the secretory pathways of the host cell used for expression of the RNA may include, but is not limited to signal peptide cleavage; formation of disulfide bonds; proper folding; addition and processing of carbohydrates (such as for example, N- and O-linked glycosylation); specific proteolytic cleavages; and/or assembly into multimeric proteins. An albumin fusion protein is preferably encoded by RNA in a non-processed form which in particular has a signal peptide at its N-terminus and following secretion by a cell is preferably present in the processed form wherein in particular the signal peptide has been cleaved off. In a most preferred embodiment, the “processed form of an albumin fusion protein” refers to an albumin fusion protein product which has undergone N-terminal signal peptide cleavage, herein also referred to as a “mature albumin fusion protein”.

In preferred embodiments, albumin fusion proteins comprising a therapeutic protein have a higher plasma stability compared to the plasma stability of the same therapeutic protein when not fused to albumin. Plasma stability typically refers to the time period between when the therapeutic protein is administered in vivo and carried into the bloodstream and when the therapeutic protein is degraded and cleared from the bloodstream, into an organ, such as the kidney or liver that ultimately clears the therapeutic protein from the body. Plasma stability is calculated in terms of the half-life of the therapeutic protein in the bloodstream. The half-life of the therapeutic protein in the bloodstream can be readily determined by common assays known in the art.

As used herein, “albumin” refers collectively to albumin protein or amino acid sequence, or an albumin fragment or variant, having one or more functional activities (e.g., biological activities) of albumin. In particular, “albumin” refers to human albumin or fragments or variants thereof especially the mature form of human albumin, or albumin from other vertebrates or fragments thereof, or variants of these molecules. The albumin may be derived from any vertebrate, especially any mammal, for example human, mouse, cow, sheep, or pig. Non-mammalian albumins include, but are not limited to, hen and salmon. The albumin portion of the albumin fusion protein may be from a different animal than the therapeutic protein portion.

In certain embodiments, the albumin is human serum albumin (HSA), or fragments or variants thereof, such as those disclosed in U.S. Pat. No. 5,876,969, WO 2011/124718, WO 2013/075066, and WO 2011/0514789.

The terms, human serum albumin (HSA) and human albumin (HA) are used interchangeably herein. The terms, “albumin and “serum albumin” are broader, and encompass human serum albumin (and fragments and variants thereof) as well as albumin from other species (and fragments and variants thereof).

As used herein, a fragment of albumin sufficient to prolong the therapeutic activity or plasma stability of the therapeutic protein refers to a fragment of albumin sufficient in length or structure to stabilize or prolong the therapeutic activity or plasma stability of the protein so that the plasma stability of the therapeutic protein portion of the albumin fusion protein is prolonged or extended compared to the plasma stability in the non-fusion state.

The albumin portion of the albumin fusion proteins may comprise the full length of the albumin sequence, or may include one or more fragments thereof that are capable of stabilizing or prolonging the therapeutic activity or plasma stability. Such fragments may be of 10 or more amino acids in length or may include about 15, 20, 25, 30, 50, or more contiguous amino acids from the albumin sequence or may include part or all of specific domains of albumin. For instance, one or more fragments of HSA spanning the first two immunoglobulin-like domains may be used. In a preferred embodiment, the HSA fragment is the mature form of HSA.

Generally speaking, an albumin fragment or variant will be at least 100 amino acids long, preferably at least 150 amino acids long.

According to the disclosure, albumin may be naturally occurring albumin or a fragment or variant thereof. Albumin may be human albumin and may be derived from any vertebrate, especially any mammal,

Preferably, the albumin fusion protein comprises albumin as the N-terminal portion, and a therapeutic protein as the C-terminal portion. Alternatively, an albumin fusion protein comprising albumin as the C-terminal portion, and a therapeutic protein as the N-terminal portion may also be used.

In one embodiment, the therapeutic protein(s) is (are) joined to the albumin through (a) peptide linker(s). A linker peptide between the fused portions may provide greater physical separation between the moieties and thus maximize the accessibility of the therapeutic protein portion, for instance, for binding to its cognate receptor. The linker peptide may consist of amino acids such that it is flexible or more rigid. The linker sequence may be cleavable by a protease or chemically.

As used herein, the term “Fc region” refers to the portion of a native immunoglobulin formed by the respective Fc domains (or Fc moieties) of its two heavy chains. As used herein, the term “Fc domain” refers to a portion or fragment of a single immunoglobulin (Ig) heavy chain wherein the Fc domain does not comprise an Fv domain. In certain embodiments, an Fc domain begins in the hinge region just upstream of the papain cleavage site and ends at the C-terminus of the antibody. Accordingly, a complete Fc domain comprises at least a hinge domain, a CH2 domain, and a CH3 domain. In certain embodiments, an Fc domain comprises at least one of: a hinge (e.g., upper, middle, and/or lower hinge region) domain, a CH2 domain, a CH3 domain, a CH4 domain, or a variant, portion, or fragment thereof. In certain embodiments, an Fc domain comprises a complete Fc domain (i.e., a hinge domain, a CH2 domain, and a CH3 domain). In certain embodiments, an Fc domain comprises a hinge domain (or portion thereof) fused to a CH3 domain (or portion thereof). In certain embodiments, an Fc domain comprises a CH2 domain (or portion thereof) fused to a CH3 domain (or portion thereof). In certain embodiments, an Fc domain consists of a CH3 domain or portion thereof. In certain embodiments, an Fc domain consists of a hinge domain (or portion thereof) and a CH3 domain (or portion thereof). In certain embodiments, an Fc domain consists of a CH2 domain (or portion thereof) and a CH3 domain. In certain embodiments, an Fc domain consists of a hinge domain (or portion thereof) and a CH2 domain (or portion thereof). In certain embodiments, an Fc domain lacks at least a portion of a CH2 domain (e.g., all or part of a CH2 domain). An Fc domain herein generally refers to a polypeptide comprising all or part of the Fc domain of an immunoglobulin heavy-chain. This includes, but is not limited to, polypeptides comprising the entire CH1, hinge, CH2, and/or CH3 domains as well as fragments of such peptides comprising only, e.g., the hinge, CH2, and CH3 domain. The Fc domain may be derived from an immunoglobulin of any species and/or any subtype, including, but not limited to, a human IgG1, IgG2, IgG3, IgG4, IgD, IgA, IgE, or IgM antibody. The Fc domain encompasses native Fc and Fc variant molecules. As set forth herein, it will be understood by one of ordinary skill in the art that any Fc domain may be modified such that it varies in amino acid sequence from the native Fc domain of a naturally occurring immunoglobulin molecule. In certain embodiments, the Fc domain has reduced effector function (e.g., FcγR binding).

The Fc domains of a polypeptide described herein may be derived from different immunoglobulin molecules. For example, an Fc domain of a polypeptide may comprise a CH2 and/or CH3 domain derived from an IgG1 molecule and a hinge region derived from an IgG3 molecule. In another example, an Fc domain can comprise a chimeric hinge region derived, in part, from an IgG1 molecule and, in part, from an IgG3 molecule. In another example, an Fc domain can comprise a chimeric hinge derived, in part, from an IgG1 molecule and, in part, from an IgG4 molecule.

In certain embodiments, an extended-PK group includes an Fc domain or fragments thereof or variants of the Fc domain or fragments thereof (all of which for the purpose of the present disclosure are comprised by the term “Fc domain”). The Fc domain does not contain a variable region that binds to antigen. Fc domains suitable for use in the present disclosure may be obtained from a number of different sources. In certain embodiments, an Fc domain is derived from a human immunoglobulin. In certain embodiments, the Fc domain is from a human IgG1 constant region. It is understood, however, that the Fc domain may be derived from an immunoglobulin of another mammalian species, including for example, a rodent (e.g. a mouse, rat, rabbit, guinea pig) or non-human primate (e.g. chimpanzee, macaque) species.

Moreover, the Fc domain (or a fragment or variant thereof) may be derived from any immunoglobulin class, including IgM, IgG, IgD, IgA, and IgE, and any immunoglobulin isotype, including IgG1, IgG2, IgG3, and IgG4.

A variety of Fc domain gene sequences (e.g., mouse and human constant region gene sequences) are available in the form of publicly accessible deposits. Constant region domains comprising an Fc domain sequence can be selected lacking a particular effector function and/or with a particular modification to reduce immunogenicity. Many sequences of antibodies and antibody-encoding genes have been published and suitable Fc domain sequences (e.g. hinge, CH2, and/or CH3 sequences, or fragments or variants thereof) can be derived from these sequences using art recognized techniques.

In certain embodiments, the extended-PK group is a serum albumin binding protein such as those described in US2005/0287153, US2007/0003549, US2007/0178082, US2007/0269422, US2010/0113339, WO2009/083804, and WO2009/133208, which are herein incorporated by reference in their entirety. In certain embodiments, the extended-PK group is transferrin, as disclosed in U.S. Pat. Nos. 7,176,278 and 8,158,579, which are herein incorporated by reference in their entirety. In certain embodiments, the extended-PK group is a serum immunoglobulin binding protein such as those disclosed in US2007/0178082, US2014/0220017, and US2017/0145062, which are herein incorporated by reference in their entirety. In certain embodiments, the extended-PK group is a fibronectin (Fn)-based scaffold domain protein that binds to serum albumin, such as those disclosed in US2012/0094909, which is herein incorporated by reference in its entirety. Methods of making fibronectin-based scaffold domain proteins are also disclosed in US2012/0094909. A non-limiting example of a Fn3-based extended-PK group is Fn3(HSA), i.e., a Fn3 protein that binds to human serum albumin.

In certain aspects, the extended-PK cytokine, suitable for use according to the disclosure, can employ one or more peptide linkers. As used herein, the term “peptide linker” refers to a peptide or polypeptide sequence which connects two or more domains (e.g., the extended-PK moiety and an IL moiety such as IL2) in a linear amino acid sequence of a polypeptide chain. For example, peptide linkers may be used to connect a cytokine moiety to a HSA domain.

Linkers suitable for fusing the extended-PK group to e.g. IL2 are well known in the art. Exemplary linkers include glycine-serine-polypeptide linkers, glycine-proline-polypeptide linkers, and proline-alanine polypeptide linkers. In certain embodiments, the linker is a glycine-serine-polypeptide linker, i.e., a peptide that consists of glycine and serine residues.

In addition to, or in place of, the heterologous polypeptides described above, a cytokine variant polypeptide described herein can contain sequences encoding a “marker” or “reporter”. Examples of marker or reporter genes include β-lactamase, chloramphenicol acetyltransferase (CAT), adenosine deaminase (ADA), aminoglycoside phosphotransferase, dihydrofolate reductase (DHFR), hygromycin-B-hosphotransferase (HPH), thymidine kinase (TK), β-galactosidase, and xanthine guanine phosphoribosyltransferase (XGPRT).

### Antigen

The methods described herein may further comprise the step of contacting the immune effector cells, in particular immune effector cells expressing an antigen receptor, e.g., immune effector cells which are genetically manipulated to express an antigen receptor, in the subject being treated, with a cognate antigen molecule (also referred herein to as “antigen targeted by the antigen receptor”, “vaccine antigen” or simply “antigen”), wherein the antigen molecule or a procession product thereof, e.g., a fragment thereof, binds to the antigen receptor such as TCR or CAR carried by the immune effector cells. In one embodiment, the cognate antigen molecule comprises the antigen expressed by a target cell to which the immune effector cells are targeted or a fragment thereof, or a variant of the antigen or the fragment.

Accordingly, the methods described herein comprise the step of administering the cognate antigen molecule, a nucleic acid coding therefor or cells expressing the cognate antigen molecule to the subject. In one embodiment, the nucleic acid encoding the cognate antigen molecule is expressed in cells of the subject to provide the cognate antigen molecule. In one embodiment, expression of the cognate antigen molecule is at the cell surface. In one embodiment, the nucleic acid encoding the cognate antigen molecule is transiently expressed in cells of the subject. In one embodiment, the nucleic encoding the cognate antigen molecule is RNA. In one embodiment, the cognate antigen molecule or the nucleic acid coding therefor is administered systemically. In one embodiment, after systemic administration of the nucleic acid encoding the cognate antigen molecule, expression of the nucleic acid encoding the cognate antigen molecule in spleen occurs. In one embodiment, after systemic administration of the nucleic acid encoding the cognate antigen molecule, expression of the nucleic acid encoding the cognate antigen molecule in antigen presenting cells, preferably professional antigen presenting cells occurs. In one embodiment, the antigen presenting cells are selected from the group consisting of dendritic cells, macrophages and B cells. In one embodiment, after systemic administration of the nucleic acid encoding the cognate antigen molecule, no or essentially no expression of the nucleic acid encoding the cognate antigen molecule in lung and/or liver occurs. In one embodiment, after systemic administration of the nucleic acid encoding the cognate antigen molecule, expression of the nucleic acid encoding the cognate antigen molecule in spleen is at least 5-fold the amount of expression in lung.

A peptide and protein antigen which is provided to a subject according to the invention (either by administering the peptide and protein antigen, a nucleic acid, in particular RNA, encoding the peptide and protein antigen or cells expressing the peptide and protein antigen), i.e., a vaccine antigen, preferably results in stimulation, priming and/or expansion of immune effector cells in the subject being administered the peptide or protein antigen, nucleic acid or cells. Said stimulated, primed and/or expanded immune effector cells are preferably directed against a target antigen, in particular a target antigen expressed by diseased cells, tissues and/or organs, i.e., a disease-associated antigen. Thus, a vaccine antigen may comprise the disease-associated antigen, or a fragment or variant thereof. In one embodiment, such fragment or variant is immunologically equivalent to the disease-associated antigen. In the context of the present disclosure, the term “fragment of an antigen” or “variant of an antigen” means an agent which results in stimulation, priming and/or expansion of immune effector cells which stimulated, primed and/or expanded immune effector cells target the antigen, i.e. a disease-associated antigen, in particular when presented by diseased cells, tissues and/or organs. Thus, the vaccine antigen may correspond to or may comprise the disease-associated antigen, may correspond to or may comprise a fragment of the disease-associated antigen or may correspond to or may comprise an antigen which is homologous to the disease-associated antigen or a fragment thereof. If the vaccine antigen comprises a fragment of the disease-associated antigen or an amino acid sequence which is homologous to a fragment of the disease-associated antigen said fragment or amino acid sequence may comprise an epitope of the disease-associated antigen to which the antigen receptor of the immune effector cells is targeted or a sequence which is homologous to an epitope of the disease-associated antigen. Thus, according to the disclosure, a vaccine antigen may comprise an immunogenic fragment of a disease-associated antigen or an amino acid sequence being homologous to an immunogenic fragment of a disease-associated antigen. An “immunogenic fragment of an antigen” according to the disclosure preferably relates to a fragment of an antigen which is capable of stimulating, priming and/or expanding immune effector cells carrying an antigen receptor binding to the antigen or cells expressing the antigen. It is preferred that the vaccine antigen (similar to the disease-associated antigen) provides the relevant epitope for binding by the antigen binding domain present in the immune effector cells. In one embodiment, the vaccine antigen (similar to the disease-associated antigen) is expressed on the surface of a cell such as an antigen-presenting cell so as to provide the relevant epitope for binding by immune effector cells. In one embodiment, the vaccine antigen (similar to the disease-associated antigen) is expressed by and presented on the surface of a cell such as an antigen-presenting cell in the context of MHC so as to provide the relevant epitope for binding by immune effector cells. The vaccine antigen may be a recombinant antigen.

In one embodiment of all aspects of the invention, the nucleic acid encoding the vaccine antigen is expressed in cells of a subject to provide the antigen or a procession product thereof for binding by the antigen receptor expressed by immune effector cells, said binding resulting in stimulation, priming and/or expansion of the immune effector cells.

The term “immunologically equivalent” means that the immunologically equivalent molecule such as the immunologically equivalent amino acid sequence exhibits the same or essentially the same immunological properties and/or exerts the same or essentially the same immunological effects, e.g., with respect to the type of the immunological effect. In the context of the present disclosure, the term “immunologically equivalent” is preferably used with respect to the immunological effects or properties of antigens or antigen variants used for immunization. For example, an amino acid sequence is immunologically equivalent to a reference amino acid sequence if said amino acid sequence when exposed to the immune system of a subject such as T cells binding to the reference amino acid sequence or cells expressing the reference amino acid sequence induces an immune reaction having a specificity of reacting with the reference amino acid sequence. Thus, a molecule which is immunologically equivalent to an antigen exhibits the same or essentially the same properties and/or exerts the same or essentially the same effects regarding the stimulation, priming and/or expansion of T cells as the antigen to which the T cells are targeted.

“Activation” or “stimulation”, as used herein, refers to the state of an immune effector cell such as T cell that has been sufficiently stimulated to induce detectable cellular proliferation. Activation can also be associated with initiation of signaling pathways, induced cytokine production, and detectable effector functions. The term “activated immune effector cells” refers to, among other things, immune effector cells that are undergoing cell division.

The term “priming” refers to a process wherein an immune effector cell such as a T cell has its first contact with its specific antigen and causes differentiation into effector cells such as effector T cells.

The term “clonal expansion” or “expansion” refers to a process wherein a specific entity is multiplied. In the context of the present disclosure, the term is preferably used in the context of an immunological response in which lymphocytes are stimulated by an antigen, proliferate, and the specific lymphocyte recognizing said antigen is amplified. Preferably, clonal expansion leads to differentiation of the lymphocytes.

The term “antigen” relates to an agent comprising an epitope against which an immune response can be generated. The term “antigen” includes, in particular, proteins and peptides. In one embodiment, an antigen is presented or present on the surface of cells of the immune system such as antigen presenting cells like dendritic cells or macrophages. An antigen or a procession product thereof such as a T cell epitope is in one embodiment bound by an antigen receptor. Accordingly, an antigen or a procession product thereof may react specifically with immune effector cells such as T-lymphocytes (T cells). In one embodiment, an antigen is a disease-associated antigen, such as a tumor antigen, a viral antigen, or a bacterial antigen and an epitope is derived from such antigen.

The term “disease-associated antigen” is used in its broadest sense to refer to any antigen associated with a disease. A disease-associated antigen is a molecule which contains epitopes that will stimulate a host's immune system to make a cellular antigen-specific immune response and/or a humoral antibody response against the disease. The disease-associated antigen or an epitope thereof may therefore be used for therapeutic purposes. Disease-associated antigens may be associated with infection by microbes, typically microbial antigens, or associated with cancer, typically tumors.

The term “tumor antigen” or “tumor-associated antigen” refers to a constituent of cancer cells which may be derived from the cytoplasm, the cell surface and the cell nucleus. In particular, it refers to those antigens which are produced intracellularly or as surface antigens on tumor cells. A tumor antigen is typically expressed preferentially by cancer cells (e.g., it is expressed at higher levels in cancer cells than in non-cancer cells) and in some instances it is expressed solely by cancer cells. Examples of tumor antigens include, without limitation, p53, ART-4, BAGE, beta-catenin/m, Bcr-abL CAMEL, CAP-1, CASP-8, CDC27/m, CDK4/m, CEA, the cell surface proteins of the claudin family, such as CLAUDIN-6, CLAUDIN-18.2 and CLAUDIN-12, c-MYC, CT, Cyp-B, DAM, ELF2M, ETV6-AML1, G250, GAGE, GnT-V, Gap 100, HAGE, HER-2/neu, HPV-E7, HPV-E6, HAST-2, hTERT (or hTRT), LAGE, LDLR/FUT, MAGE-A, preferably MAGE-A1, MAGE-A2, MAGE-A3, MAGE-A4, MAGE-A5, MAGE-A6, MAGE-A7, MAGE-A8, MAGE-A9, MAGE-A 10, MAGE-A 1 1, or MAGE-A12, MAGE-B, MAGE-C, MART-1/Melan-A, MCI R, Myosin/m, MUC1, MUM-1, MUM-2, MUM-3, NA88-A, NF1, NY-ESO-1, NY-BR-1, p190 minor BCR-abL, Pml/RARa, PRAME, proteinase 3, PSA, PSM, RAGE, RU1 or RU2, SAGE, SART-1 or SART-3, SCGB3A2, SCP1, SCP2, SCP3, SSX, SURVIVIN, TEL/AML1, TPI/m, TRP-1, TRP-2, TRP-2/INT2, TPTE, WT, and WT-1. Particularly, preferred tumor antigens are proteins of the claudin family, such as CLAUDIN-6 or CLAUDIN-18.2.

The term “viral antigen” refers to any viral component having antigenic properties, i.e. being able to provoke an immune response in an individual. The viral antigen may be a viral ribonucleoprotein or an envelope protein.

The term “bacterial antigen” refers to any bacterial component having antigenic properties, i.e. being able to provoke an immune response in an individual. The bacterial antigen may be derived from the cell wall or cytoplasm membrane of the bacterium.

The term “expressed on the cell surface” or “associated with the cell surface” means that a molecule such as a receptor or antigen is associated with and located at the plasma membrane of a cell, wherein at least a part of the molecule faces the extracellular space of said cell and is accessible from the outside of said cell, e.g., by antibodies located outside the cell. In this context, a part is preferably at least 4, preferably at least 8, preferably at least 12, more preferably at least 20 amino acids. The association may be direct or indirect. For example, the association may be by one or more transmembrane domains, one or more lipid anchors, or by the interaction with any other protein, lipid, saccharide, or other structure that can be found on the outer leaflet of the plasma membrane of a cell. For example, a molecule associated with the surface of a cell may be a transmembrane protein having an extracellular portion or may be a protein associated with the surface of a cell by interacting with another protein that is a transmembrane protein.

“Cell surface” or “surface of a cell” is used in accordance with its normal meaning in the art, and thus includes the outside of the cell which is accessible to binding by proteins and other molecules. An antigen is expressed on the surface of cells if it is located at the surface of said cells and is accessible to binding by e.g. antigen-specific antibodies added to the cells. In one embodiment, an antigen expressed on the surface of cells is an integral membrane protein having an extracellular portion recognized by a CAR.

The term “extracellular portion” or “exodomain” in the context of the present invention refers to a part of a molecule such as a protein that is facing the extracellular space of a cell and preferably is accessible from the outside of said cell, e.g., by binding molecules such as antibodies located outside the cell. Preferably, the term refers to one or more extracellular loops or domains or a fragment thereof.

The term “epitope” refers to a part or fragment of a molecule such as an antigen that is recognized by the immune system. For example, the epitope may be recognized by T cells, B cells or antibodies. An epitope of an antigen may include a continuous or discontinuous portion of the antigen and may be between about 5 and about 100, such as between about 5 and about 50, more preferably between about 8 and about 30, most preferably between about 10 and about 25 amino acids in length, for example, the epitope may be preferably 9, 10, 11, 12, 13, 14, 15, 16, 17, 18, 19, 20, 21, 22, 23, 24, or 25 amino acids in length. In one embodiment, an epitope is between about 10 and about 25 amino acids in length. The term “epitope” includes T cell epitopes.

The term “T cell epitope” refers to a part or fragment of a protein that is recognized by a T cell when presented in the context of MHC molecules. The term “major histocompatibility complex” and the abbreviation “MHC” includes MHC class I and MHC class II molecules and relates to a complex of genes which is present in all vertebrates. MHC proteins or molecules are important for signaling between lymphocytes and antigen presenting cells or diseased cells in immune reactions, wherein the MHC proteins or molecules bind peptide epitopes and present them for recognition by T cell receptors on T cells. The proteins encoded by the MHC are expressed on the surface of cells, and display both self-antigens (peptide fragments from the cell itself) and non-self-antigens (e.g., fragments of invading microorganisms) to a T cell. In the case of class I MHC/peptide complexes, the binding peptides are typically about 8 to about 10 amino acids long although longer or shorter peptides may be effective. In the case of class II MHC/peptide complexes, the binding peptides are typically about 10 to about 25 amino acids long and are in particular about 13 to about 18 amino acids long, whereas longer and shorter peptides may be effective.

In one embodiment, the target antigen is a tumor antigen and the vaccine antigen or a fragment thereof (e.g., an epitope) is derived from the tumor antigen. The tumor antigen may be a “standard” antigen, which is generally known to be expressed in various cancers. The tumor antigen may also be a “neo-antigen”, which is specific to an individual's tumor and has not been previously recognized by the immune system, A neo-antigen or neo-epitope may result from one or more cancer-specific mutations in the genome of cancer cells resulting in amino acid changes. If the tumor antigen is a neo-antigen, the vaccine antigen preferably comprises an epitope or a fragment of said neo-antigen comprising one or more amino acid changes.

Cancer mutations vary with each individual. Thus, cancer mutations that encode novel epitopes (neo-epitopes) represent attractive targets in the development of vaccine compositions and immunotherapies. The efficacy of tumor immunotherapy relies on the selection of cancer-specific antigens and epitopes capable of inducing a potent immune response within a host. RNA can be used to deliver patient-specific tumor epitopes to a patient. Dendritic cells (DCs) residing in the spleen represent antigen-presenting cells of particular interest for RNA expression of immunogenic epitopes or antigens such as tumor epitopes. The use of multiple epitopes has been shown to promote therapeutic efficacy in tumor vaccine compositions. Rapid sequencing of the tumor mutanome may provide multiple epitopes for individualized vaccines which can be encoded by RNA described herein, e.g., as a single polypeptide wherein the epitopes are optionally separated by linkers. In certain embodiments of the present disclosure, the RNA encodes at least one epitope, at least two epitopes, at least three epitopes, at least four epitopes, at least five epitopes, at least six epitopes, at least seven epitopes, at least eight epitopes, at least nine epitopes, or at least ten epitopes. Exemplary embodiments include RNA that encodes at least five epitopes (termed a “pentatope”) and RNA that encodes at least ten epitopes (termed a “decatope”).

According to the various aspects of the invention, the aim is preferably to provide an immune response against cancer cells expressing a tumor antigen such as CLDN6 or CLDN18.2 and to treat a cancer disease involving cells expressing a tumor antigen such as CLDN6 or CLDN18.2. Preferably the invention involves the administration of antigen receptor-engineered immune effector cells such as T cells targeted against cancer cells expressing a tumor antigen such as CLDN6 or CLDN18.2.

The peptide and protein antigen can be 2-100 amino acids, including for example, 5 amino acids, 10 amino acids, 15 amino acids, 20 amino acids, 25 amino acids, 30 amino acids, 35 amino acids, 40 amino acids, 45 amino acids, or 50 amino acids in length. In some embodiments, a peptide can be greater than 50 amino acids. In some embodiments, the peptide can be greater than 100 amino acids.

According to the invention, the vaccine antigen should be recognizable by an immune effector cell. Preferably, the antigen if recognized by an immune effector cell is able to induce in the presence of appropriate co-stimulatory signals, stimulation, priming and/or expansion of the immune effector cell carrying an antigen receptor recognizing the antigen. In the context of the embodiments of the present invention, the antigen is preferably present on the surface of a cell, preferably an antigen presenting cell. Recognition of the antigen on the surface of a diseased cell may result in an immune reaction against the antigen (or cell expressing the antigen).

In one embodiment of all aspects of the invention, an antigen is expressed in a diseased cell such as a cancer cell. In one embodiment, an antigen is expressed on the surface of a diseased cell such as a cancer cell. In one embodiment, an antigen receptor is a CAR which binds to an extracellular domain or to an epitope in an extracellular domain of an antigen. In one embodiment, a CAR binds to native epitopes of an antigen present on the surface of living cells. In one embodiment, binding of a CAR when expressed by T cells and/or present on T cells to an antigen present on cells such as antigen presenting cells results in stimulation, priming and/or expansion of said T cells. In one embodiment, binding of a CAR when expressed by T cells and/or present on T cells to an antigen present on diseased cells such as cancer cells results in cytolysis and/or apoptosis of the diseased cells, wherein said T cells preferably release cytotoxic factors, e.g. perforins and granzymes.

### Chemotherapy

In certain embodiments, additional treatments may be administered to a patient in combination with the treatments described herein. Such additional treatments includes classical cancer therapy, e.g., radiation therapy, surgery, hyperthermia therapy and/or chemotherapy.

Chemotherapy is a type of cancer treatment that uses one or more anti-cancer drugs (chemotherapeutic agents), usually as part of a standardized chemotherapy regimen. The term chemotherapy has come to connote non-specific usage of intracellular poisons to inhibit mitosis. The connotation excludes more selective agents that block extracellular signals (signal transduction). The development of therapies with specific molecular or genetic targets, which inhibit growth-promoting signals from classic endocrine hormones (primarily estrogens for breast cancer and androgens for prostate cancer) are now called hormonal therapies. By contrast, other inhibitions of growth-signals like those associated with receptor tyrosine kinases are referred to as targeted therapy.

Importantly, the use of drugs (whether chemotherapy, hormonal therapy or targeted therapy) constitutes systemic therapy for cancer in that they are introduced into the blood stream and are therefore in principle able to address cancer at any anatomic location in the body. Systemic therapy is often used in conjunction with other modalities that constitute local therapy (i.e. treatments whose efficacy is confined to the anatomic area where they are applied) for cancer such as radiation therapy, surgery or hyperthermia therapy.

Traditional chemotherapeutic agents are cytotoxic by means of interfering with cell division (mitosis) but cancer cells vary widely in their susceptibility to these agents. To a large extent, chemotherapy can be thought of as a way to damage or stress cells, which may then lead to cell death if apoptosis is initiated.

Chemotherapeutic agents include alkylating agents, antimetabolites, anti-microtubule agents, topoisomerase inhibitors, and cytotoxic antibiotics.

Alkylating agents have the ability to alkylate many molecules, including proteins, RNA and DNA. The subtypes of alkylating agents are the nitrogen mustards, nitrosoureas, tetrazines, aziridines, cisplatins and derivatives, and non-classical alkylating agents. Nitrogen mustards include mechlorethamine, cyclophosphamide, melphalan, chlorambucil, ifosfamide and busulfan. Nitrosoureas include N-Nitroso-N-methylurea (MNU), carmustine (BCNU), lomustine (CCNU) and semustine (MeCCNU), fotemustine and streptozotocin. Tetrazines include dacarbazine, mitozolomide and temozolomide. Aziridines include thiotepa, mytomycin and diaziquone (AZQ). Cisplatin and derivatives include cisplatin, carboplatin and oxaliplatin. They impair cell function by forming covalent bonds with the amino, carboxyl, sulfhydryl, and phosphate groups in biologically important molecules. Non-classical alkylating agents include procarbazine and hexamethylmelamine. In one particularly preferred embodiment, the alkylating agent is cyclophosphamide.

Anti-metabolites are a group of molecules that impede DNA and RNA synthesis. Many of them have a similar structure to the building blocks of DNA and RNA. Anti-metabolites resemble either nucleobases or nucleosides, but have altered chemical groups. These drugs exert their effect by either blocking the enzymes required for DNA synthesis or becoming incorporated into DNA or RNA. Subtypes of the anti-metabolites are the anti-folates, fluoropyrimidines, deoxynucleoside analogues and thiopurines. The anti-folates include methotrexate and pemetrexed. The fluoropyrimidines include fluorouracil and capecitabine. The deoxynucleoside analogues include cytarabine, gemcitabine, decitabine, azacitidine, fludarabine, nelarabine, cladribine, clofarabine, and pentostatin. The thiopurines include thioguanine and mercaptopurine.

Anti-microtubule agents block cell division by preventing microtubule function. The vinca alkaloids prevent the formation of the microtubules, whereas the taxanes prevent the microtubule disassembly. Vinca alkaloids include vinorelbine, vindesine, and vinflunine. Taxanes include docetaxel (Taxotere) and paclitaxel (Taxol).

Topoisomerase inhibitors are drugs that affect the activity of two enzymes: topoisomerase I and topoisomerase II and include irinotecan, topotecan, camptothecin, etoposide, doxorubicin, mitoxantrone, teniposide, novobiocin, merbarone, and aclarubicin.

The cytotoxic antibiotics are a varied group of drugs that have various mechanisms of action. The common theme that they share in their chemotherapy indication is that they interrupt cell division. The most important subgroup is the anthracyclines (e.g., doxorubicin, daunorubicin, epirubicin, idarubicin pirarubicin, and aclarubicin) and the bleomycins; other prominent examples include mitomycin C, mitoxantrone, and actinomycin.

In one embodiment, prior to administration of immune effector cells, a lymphodepleting treatment may be applied, e.g., by administering cyclophosphamide and fludarabine. Such treatment may increase cell persistence and the incidence and duration of clinical responses.

### Immune Checkpoint Inhibitors

In certain embodiments, immune checkpoint inhibitors are used in combination with other therapeutic agents described herein.

As used herein, “immune checkpoint” refers to co-stimulatory and inhibitory signals that regulate the amplitude and quality of T cell receptor recognition of an antigen. In certain embodiments, the immune checkpoint is an inhibitory signal. In certain embodiments, the inhibitory signal is the interaction between PD-1 and PD-L1. In certain embodiments, the inhibitory signal is the interaction between CTLA-4 and CD80 or CD86 to displace CD28 binding. In certain embodiments the inhibitory signal is the interaction between LAG3 and MHC class II molecules. In certain embodiments, the inhibitory signal is the interaction between TIM3 and galectin 9.

As used herein, “immune checkpoint inhibitor” refers to a molecule that totally or partially reduces, inhibits, interferes with or modulates one or more checkpoint proteins. In certain embodiments, the immune checkpoint inhibitor prevents inhibitory signals associated with the immune checkpoint. In certain embodiments, the immune checkpoint inhibitor is an antibody, or fragment thereof that disrupts inhibitory signaling associated with the immune checkpoint. In certain embodiments, the immune checkpoint inhibitor is a small molecule that disrupts inhibitory signaling. In certain embodiments, the immune checkpoint inhibitor is an antibody, fragment thereof, or antibody mimic, that prevents the interaction between checkpoint blocker proteins, e.g., an antibody, or fragment thereof, that prevents the interaction between PD-1 and PD-L1. In certain embodiments, the immune checkpoint inhibitor is an antibody, or fragment thereof, that prevents the interaction between CTLA-4 and CD80 or CD86. In certain embodiments, the immune checkpoint inhibitor is an antibody, or fragment thereof, that prevents the interaction between LAG3 and its ligands, or TIM-3 and its ligands. The checkpoint inhibitor may also be in the form of the soluble form of the molecules (or variants thereof) themselves, e.g., a soluble PD-L1 or PD-L1 fusion.

The “Programmed Death-1 (PD-1)” receptor refers to an immuno-inhibitory receptor belonging to the CD28 family. PD-1 is expressed predominantly on previously activated T cells in vivo, and binds to two ligands, PD-L1 and PD-L2. The term “PD-1” as used herein includes human PD-1 (hPD-1), variants, isoforms, and species homologs of hPD-1, and analogs having at least one common epitope with hPD-1.

“Programmed Death Ligand-1 (PD-L1)” is one of two cell surface glycoprotein ligands for PD-1 (the other being PD-L2) that downregulates T cell activation and cytokine secretion upon binding to PD-1. The term “PD-L1” as used herein includes human PD-L1 (hPD-L1), variants, isoforms, and species homologs of hPD-L1, and analogs having at least one common epitope with hPD-L1.

“Cytotoxic T Lymphocyte Associated Antigen-4 (CTLA-4)” is a T cell surface molecule and is a member of the immunoglobulin superfamily. This protein downregulates the immune system by binding to CD80 and CD86. The term “CTLA-4” as used herein includes human CTLA-4 (hCTLA-4), variants, isoforms, and species homologs of hCTLA-4, and analogs having at least one common epitope with hCTLA-4.

“Lymphocyte Activation Gene-3 (LAG3)” is an inhibitory receptor associated with inhibition of lymphocyte activity by binding to MHC class II molecules. This receptor enhances the function of Treg cells and inhibits CD8⁺ effector T cell function. The term “LAG3” as used herein includes human LAG3 (hLAG3), variants, isoforms, and species homologs of hLAG3, and analogs having at least one common epitope.

“T Cell Membrane Protein-3 (TIM3)” is an inhibitory receptor involved in the inhibition of lymphocyte activity by inhibition of TH1 cell responses. Its ligand is galectin 9, which is upregulated in various types of cancers. The term “TIM3” as used herein includes human TIM3 (hTIM3), variants, isoforms, and species homologs of hTIM3, and analogs having at least one common epitope.

The “B7 family” refers to inhibitory ligands with undefined receptors. The B7 family encompasses B7-H3 and B7-H4, both upregulated on tumor cells and tumor infiltrating cells.

In certain embodiments, the immune checkpoint inhibitor suitable for use in the methods disclosed herein, is an antagonist of inhibitory signals, e.g., an antibody which targets, for example, PD-1, PD-L1, CTLA-4, LAG3, B7-H3, B7-H4, or TIM3. These ligands and receptors are reviewed in Pardoll, D., Nature. 12: 252-264, 2012.

In certain embodiments, the immune checkpoint inhibitor is an antibody or an antigen-binding portion thereof, that disrupts or inhibits signaling from an inhibitory immunoregulator. In certain embodiments, the immune checkpoint inhibitor is a small molecule that disrupts or inhibits signaling from an inhibitory immunoregulator.

In certain embodiments, the inhibitory immunoregulator is a component of the PD-1/PD-L1 signaling pathway. Accordingly, certain embodiments of the disclosure provide for administering to a subject an antibody or an antigen-binding portion thereof that disrupts the interaction between the PD-1 receptor and its ligand, PD-L1. Antibodies which bind to PD-1 and disrupt the interaction between the PD-1 and its ligand, PD-L1, are known in the art. In certain embodiments, the antibody or antigen-binding portion thereof binds specifically to PD-1. In certain embodiments, the antibody or antigen-binding portion thereof binds specifically to PD-L1 and inhibits its interaction with PD-1, thereby increasing immune activity.

In certain embodiments, the inhibitory immunoregulator is a component of the CTLA4 signaling pathway. Accordingly, certain embodiments of the disclosure provide for administering to a subject an antibody or an antigen-binding portion thereof that targets CTLA4 and disrupts its interaction with CD80 and CD86.

In certain embodiments, the inhibitory immunoregulator is a component of the LAG3 (lymphocyte activation gene 3) signaling pathway. Accordingly, certain embodiments of the disclosure provide for administering to a subject an antibody or an antigen-binding portion thereof that targets LAG3 and disrupts its interaction with MHC class II molecules.

In certain embodiments, the inhibitory immunoregulator is a component of the B7 family signaling pathway. In certain embodiments, the B7 family members are B7-H3 and B7-H4. Accordingly, certain embodiments of the disclosure provide for administering to a subject an antibody or an antigen-binding portion thereof that targets B7-H3 or H4. The B7 family does not have any defined receptors but these ligands are upregulated on tumor cells or tumor-infiltrating cells. Preclinical mouse models have shown that blockade of these ligands can enhance anti-tumor immunity.

In certain embodiments, the inhibitory immunoregulator is a component of the TIM3 (T cell membrane protein 3) signaling pathway. Accordingly, certain embodiments of the disclosure provide for administering to a subject an antibody or an antigen-binding portion thereof that targets TIM3 and disrupts its interaction with galectin 9.

It will be understood by one of ordinary skill in the art that other immune checkpoint targets can also be targeted by antagonists or antibodies, provided that the targeting results in the stimulation of an immune response such as an anti-tumor immune response as reflected in, e.g., an increase in T cell proliferation, enhanced T cell activation, and/or increased cytokine production (e.g., IFN-γ, IL2).

### RNA Targeting

It is particularly preferred according to the invention that the peptides, proteins or polypeptides described herein, in particular the vaccine antigens, are administered in the form of RNA encoding the peptides, proteins or polypeptides described herein. In one embodiment, different peptides, proteins or polypeptides described herein are encoded by different RNA molecules.

In one embodiment, the RNA is formulated in a delivery vehicle. In one embodiment, the delivery vehicle comprises particles. In one embodiment, the delivery vehicle comprises at least one lipid. In one embodiment, the at least one lipid comprises at least one cationic lipid. In one embodiment, the lipid forms a complex with and/or encapsulates the RNA. In one embodiment, the lipid is comprised in a vesicle encapsulating the RNA. In one embodiment, the RNA is formulated in liposomes.

According to the disclosure, after administration of the RNA described herein, at least a portion of the RNA is delivered to a target cell. In one embodiment, at least a portion of the RNA is delivered to the cytosol of the target cell. In one embodiment, the RNA is translated by the target cell to produce the encoded peptide or protein.

Some aspects of the disclosure involve the targeted delivery of the RNA disclosed herein (e.g., RNA encoding vaccine antigen).

In one embodiment, the disclosure involves targeting the lymphatic system, in particular secondary lymphoid organs, more specifically spleen. Targeting the lymphatic system, in particular secondary lymphoid organs, more specifically spleen is in particular preferred if the RNA administered is RNA encoding vaccine antigen.

In one embodiment, the target cell is a spleen cell. In one embodiment, the target cell is an antigen presenting cell such as a professional antigen presenting cell in the spleen. In one embodiment, the target cell is a dendritic cell in the spleen.

The “lymphatic system” is part of the circulatory system and an important part of the immune system, comprising a network of lymphatic vessels that carry lymph. The lymphatic system consists of lymphatic organs, a conducting network of lymphatic vessels, and the circulating lymph. The primary or central lymphoid organs generate lymphocytes from immature progenitor cells. The thymus and the bone marrow constitute the primary lymphoid organs. Secondary or peripheral lymphoid organs, which include lymph nodes and the spleen, maintain mature naïve lymphocytes and initiate an adaptive immune response.

RNA may be delivered to spleen by so-called lipoplex formulations, in which the RNA is bound to liposomes comprising a cationic lipid and optionally an additional or helper lipid to form injectable nanoparticle formulations. The liposomes may be obtained by injecting a solution of the lipids in ethanol into water or a suitable aqueous phase. RNA lipoplex particles may be prepared by mixing the liposomes with RNA. Spleen targeting RNA lipoplex particles are described in WO 2013/143683, herein incorporated by reference. It has been found that RNA lipoplex particles having a net negative charge may be used to preferentially target spleen tissue or spleen cells such as antigen-presenting cells, in particular dendritic cells. Accordingly, following administration of the RNA lipoplex particles, RNA accumulation and/or RNA expression in the spleen occurs. Thus, RNA lipoplex particles of the disclosure may be used for expressing RNA in the spleen. In an embodiment, after administration of the RNA lipoplex particles, no or essentially no RNA accumulation and/or RNA expression in the lung and/or liver occurs. In one embodiment, after administration of the RNA lipoplex particles, RNA accumulation and/or RNA expression in antigen presenting cells, such as professional antigen presenting cells in the spleen occurs. Thus, RNA lipoplex particles of the disclosure may be used for expressing RNA in such antigen presenting cells. In one embodiment, the antigen presenting cells are dendritic cells and/or macrophages.

In the context of the present disclosure, the term “RNA lipoplex particle” relates to a particle that contains lipid, in particular cationic lipid, and RNA. Such cationic lipids are described above. Electrostatic interactions between positively charged liposomes and negatively charged RNA results in complexation and spontaneous formation of RNA lipoplex particles. Positively charged liposomes may be generally synthesized using a cationic lipid, such as DOTMA, and additional lipids, such as DOPE. In one embodiment, a RNA lipoplex particle is a nanoparticle.

An additional lipid may be incorporated to adjust the overall positive to negative charge ratio and physical stability of the RNA lipoplex particles. Such additional lipids are described above. In certain embodiments, the additional lipid is a neutral lipid. As used herein, a “neutral lipid” refers to a lipid having a net charge of zero. Examples of neutral lipids include, but are not limited to, 1,2-di-(9Z-octadecenoyl)-sn-glycero-3-phosphoethanolamine (DOPE), 1,2-dioleoyl-sn-glycero-3-phosphocholine (DOPC), diacylphosphatidyl choline, diacylphosphatidyl ethanol amine, ceramide, sphingoemyelin, cephalin, cholesterol, and cerebroside. In specific embodiments, the additional lipid is DOPE, cholesterol and/or DOPC.

In certain embodiments, the RNA lipoplex particles include both a cationic lipid and an additional lipid. In an exemplary embodiment, the cationic lipid is DOTMA and the additional lipid is DOPE.

In some embodiments, the molar ratio of the at least one cationic lipid to the at least one additional lipid is from about 10:0 to about 1:9, about 4:1 to about 1:2, or about 3:1 to about 1:1, In specific embodiments, the molar ratio may be about 3:1, about 2.75:1, about 2.5:1, about 2.25:1, about 2:1, about 1.75:1, about 1.5:1, about 1.25:1, or about 1:1. In an exemplary embodiment, the molar ratio of the at least one cationic lipid to the at least one additional lipid is about 2:1.

RNA lipoplex particles described herein have an average diameter that in one embodiment ranges from about 200 nm to about 1000 nm, from about 200 nm to about 800 nm, from about 250 to about 700 nm, from about 400 to about 600 nm, from about 300 nm to about 500 nm, or from about 350 nm to about 400 nm. In specific embodiments, the RNA lipoplex particles have an average diameter of about 200 nm, about 225 nm, about 250 nm, about 275 nm, about 300 nm, about 325 nm, about 350 nm, about 375 nm, about 400 nm, about 425 nm, about 450 nm, about 475 nm, about 500 nm, about 525 nm, about 550 nm, about 575 nm, about 600 nm, about 625 nm, about 650 nm, about 700 nm, about 725 nm, about 750 nm, about 775 nm, about 800 nm, about 825 nm, about 850 nm, about 875 nm, about 900 nm, about 925 nm, about 950 nm, about 975 nm, or about 1000 nm. In an embodiment, the RNA lipoplex particles have an average diameter that ranges from about 250 nm to about 700 nm. In another embodiment, the RNA lipoplex particles have an average diameter that ranges from about 300 nm to about 500 nm. In an exemplary embodiment, the RNA lipoplex particles have an average diameter of about 400 nm.

The electric charge of the RNA lipoplex particles of the present disclosure is the sum of the electric charges present in the at least one cationic lipid and the electric charges present in the RNA. The charge ratio is the ratio of the positive charges present in the at least one cationic lipid to the negative charges present in the RNA. The charge ratio of the positive charges present in the at least one cationic lipid to the negative charges present in the RNA is calculated by the following equation: charge ratioqcationic lipid concentration (mol))*(the total number of positive charges in the cationic lipid)]/[(RNA concentration (mol))*(the total number of negative charges in RNA)].

The spleen targeting RNA lipoplex particles described herein at physiological pH preferably have a net negative charge such as a charge ratio of positive charges to negative charges from about 1.9:2 to about 1:2. In specific embodiments, the charge ratio of positive charges to negative charges in the RNA lipoplex particles at physiological pH is about 1.9:2.0, about 1.8:2.0, about 1.7:2.0, about 1.6:2.0, about 1.5:2.0, about 1.4:2.0, about 1.3:2.0, about 1.2:2.0, about 1.1:2.0, or about 1:2.0.

RNA delivery systems have an inherent preference to the liver. This pertains to lipid-based particles, cationic and neutral nanoparticles, in particular lipid nanoparticles such as liposomes, nanomicelles and lipophilic ligands in bioconjugates. Liver accumulation is caused by the discontinuous nature of the hepatic vasculature or the lipid metabolism (liposomes and lipid or cholesterol conjugates).

In one embodiment of the targeted delivery of a cytokine such as IL2, the target organ is liver and the target tissue is liver tissue. The delivery to such target tissue is preferred, in particular, if presence of the cytokine in this organ or tissue is desired and/or if it is desired to express large amounts of the cytokine and/or if systemic presence of the cytokine, in particular in significant amounts, is desired or required.

In one embodiment, RNA encoding a cytokine is administered in a formulation for targeting liver. Such formulations are described herein above.

For in vivo delivery of RNA to the liver, a drug delivery system may be used to transport the RNA into the liver by preventing its degradation. For example, polyplex nanomicelles consisting of a poly(ethylene glycol) (PEG)-coated surface and an mRNA-containing core is a useful system because the nanomicelles provide excellent in vivo stability of the RNA, under physiological conditions. Furthermore, the stealth property provided by the polyplex nanomicelle surface, composed of dense PEG palisades, effectively evades host immune defenses.

### Pharmaceutical Compositions

The nucleic acids, nucleic acid particles, peptides, proteins, polypeptides, RNA, RNA particles, immune effector cells and further agents, e.g., immune checkpoint inhibitors, described herein may be administered in pharmaceutical compositions or medicaments for therapeutic or prophylactic treatments and may be administered in the form of any suitable pharmaceutical composition which may comprise a pharmaceutically acceptable carrier and may optionally comprise one or more adjuvants, stabilizers etc. In one embodiment, the pharmaceutical composition is for therapeutic or prophylactic treatments, e.g., for use in treating or preventing a disease involving an antigen such as a cancer disease such as those described herein.

The term “pharmaceutical composition” relates to a formulation comprising a therapeutically effective agent, preferably together with pharmaceutically acceptable carriers, diluents and/or excipients. Said pharmaceutical composition is useful for treating, preventing, or reducing the severity of a disease or disorder by administration of said pharmaceutical composition to a subject. A pharmaceutical composition is also known in the art as a pharmaceutical formulation. In the context of the present disclosure, the pharmaceutical composition comprises nucleic acids, nucleic acid particles, peptides, proteins, polypeptides, RNA, RNA particles, immune effector cells and/or further agents as described herein.

The pharmaceutical compositions of the present disclosure may comprise one or more adjuvants or may be administered with one or more adjuvants. The term “adjuvant” relates to a compound which prolongs, enhances or accelerates an immune response. Adjuvants comprise a heterogeneous group of compounds such as oil emulsions (e.g., Freund's adjuvants), mineral compounds (such as alum), bacterial products (such as Bordetella pertussis toxin), or immune-stimulating complexes. Examples of adjuvants include, without limitation, LPS, GP96, CpG oligodeoxynucleotides, growth factors, and cytokines, such as monokines, lymphokines, interleukins, chemokines. The cytokines may be IL1, IL2, IL3, IL4, IL5, IL6, IL7, IL8, IL9, IL10, IL12, IFNα, IFNγ, GM-CSF, LT-a. Further known adjuvants are aluminium hydroxide, Freund's adjuvant or oil such as Montanide® ISA51. Other suitable adjuvants for use in the present disclosure include lipopeptides, such as Pam3Cys.

The pharmaceutical compositions according to the present disclosure are generally applied in a “pharmaceutically effective amount” and in “a pharmaceutically acceptable preparation”.

The term “pharmaceutically acceptable” refers to the non-toxicity of a material which does not interact with the action of the active component of the pharmaceutical composition.

The term “pharmaceutically effective amount” or “therapeutically effective amount” refers to the amount which achieves a desired reaction or a desired effect alone or together with further doses. In the case of the treatment of a particular disease, the desired reaction preferably relates to inhibition of the course of the disease. This comprises slowing down the progress of the disease and, in particular, interrupting or reversing the progress of the disease. The desired reaction in a treatment of a disease may also be delay of the onset or a prevention of the onset of said disease or said condition. An effective amount of the compositions described herein will depend on the condition to be treated, the severeness of the disease, the individual parameters of the patient, including age, physiological condition, size and weight, the duration of treatment, the type of an accompanying therapy (if present), the specific route of administration and similar factors. Accordingly, the doses administered of the compositions described herein may depend on various of such parameters. In the case that a reaction in a patient is insufficient with an initial dose, higher doses (or effectively higher doses achieved by a different, more localized route of administration) may be used.

The pharmaceutical compositions of the present disclosure may contain salts, buffers, preservatives, and optionally other therapeutic agents. In one embodiment, the pharmaceutical compositions of the present disclosure comprise one or more pharmaceutically acceptable carriers, diluents and/or excipients.

Suitable preservatives for use in the pharmaceutical compositions of the present disclosure include, without limitation, benzalkonium chloride, chlorobutanol, paraben and thimerosal.

The term “excipient” as used herein refers to a substance which may be present in a pharmaceutical composition of the present disclosure but is not an active ingredient. Examples of excipients, include without limitation, carriers, binders, diluents, lubricants, thickeners, surface active agents, preservatives, stabilizers, emulsifiers, buffers, flavoring agents, or colorants.

The term “diluent” relates a diluting and/or thinning agent. Moreover, the term “diluent” includes any one or more of fluid, liquid or solid suspension and/or mixing media. Examples of suitable diluents include ethanol, glycerol and water.

The term “carrier” refers to a component which may be natural, synthetic, organic, inorganic in which the active component is combined in order to facilitate, enhance or enable administration of the pharmaceutical composition. A carrier as used herein may be one or more compatible solid or liquid fillers, diluents or encapsulating substances, which are suitable for administration to subject. Suitable carrier include, without limitation, sterile water, Ringer, Ringer lactate, sterile sodium chloride solution, isotonic saline, polyalkylene glycols, hydrogenated naphthalenes and, in particular, biocompatible lactide polymers, lactide/glycolide copolymers or polyoxyethylene/polyoxy-propylene copolymers. In one embodiment, the pharmaceutical composition of the present disclosure includes isotonic saline.

Pharmaceutically acceptable carriers, excipients or diluents for therapeutic use are well known in the pharmaceutical art, and are described, for example, in Remington's Pharmaceutical Sciences, Mack Publishing Co. (A. R Gennaro edit. 1985).

Pharmaceutical carriers, excipients or diluents can be selected with regard to the intended route of administration and standard pharmaceutical practice.

In one embodiment, pharmaceutical compositions described herein may be administered intravenously, intraarterially, subcutaneously, intradermally or intramuscularly. In certain embodiments, the pharmaceutical composition is formulated for local administration or systemic administration. Systemic administration may include enteral administration, which involves absorption through the gastrointestinal tract, or parenteral administration. As used herein, “parenteral administration” refers to the administration in any manner other than through the gastrointestinal tract, such as by intravenous injection. In a preferred embodiment, the pharmaceutical compositions is formulated for systemic administration. In another preferred embodiment, the systemic administration is by intravenous administration. In one embodiment of all aspects of the invention, RNA encoding an antigen is administered systemically.

The term “co-administering” as used herein means a process whereby different compounds or compositions (e.g., immune effector cells (which may be “administered” by in vivo generation in a subject), and antigen, polynucleotide encoding antigen, or host cell genetically modified to express antigen) are administered to the same patient. The different compounds or compositions may be administered simultaneously, at essentially the same time, or sequentially. The antigen, polynucleotide encoding antigen, or host cell genetically modified to express antigen in one embodiment is administered following administration or generation of immune effector cells genetically modified to express an antigen receptor, e.g., at least one day, such as 1 to 10 days or 1 to 5 days following administration or generation of immune effector cells genetically modified to express an antigen receptor. The antigen, polynucleotide encoding antigen, or host cell genetically modified to express antigen may be administered several times over time in constant or different time intervals, e.g., following administration or generation of immune effector cells genetically modified to express an antigen receptor, e.g., in time intervals of between 10 and 40 days, wherein the first administration of antigen, polynucleotide encoding antigen, or host cell genetically modified to express antigen may be at least one day, such as 1 to 10 days or 1 to 5 days following administration or generation of immune effector cells genetically modified to express an antigen receptor.

### Treatments

The agents, compositions and methods described herein can be used to treat a subject with a disease, e.g., a disease characterized by the presence of diseased cells expressing an antigen. Particularly preferred diseases are cancer diseases. For example, if the antigen is derived from a virus, the agents, compositions and methods may be useful in the treatment of a viral disease caused by said virus. If the antigen is a tumor antigen, the agents, compositions and methods may be useful in the treatment of a cancer disease wherein cancer cells express said tumor antigen.

The agents, compositions and methods described herein may be used in the therapeutic or prophylactic treatment of various diseases, wherein provision of immune effector cells and/or activity of immune effector cells as described herein is beneficial for a patient such as cancer and infectious diseases In one embodiment, the agents, compositions and methods described herein are useful in a prophylactic and/or therapeutic treatment of a disease involving an antigen.

The term “disease” refers to an abnormal condition that affects the body of an individual. A disease is often construed as a medical condition associated with specific symptoms and signs. A disease may be caused by factors originally from an external source, such as infectious disease, or it may be caused by internal dysfunctions, such as autoimmune diseases. In humans, “disease” is often used more broadly to refer to any condition that causes pain, dysfunction, distress, social problems, or death to the individual afflicted, or similar problems for those in contact with the individual. In this broader sense, it sometimes includes injuries, disabilities, disorders, syndromes, infections, isolated symptoms, deviant behaviors, and atypical variations of structure and function, while in other contexts and for other purposes these may be considered distinguishable categories. Diseases usually affect individuals not only physically, but also emotionally, as contracting and living with many diseases can alter one's perspective on life, and one's personality.

In the present context, the term “treatment”, “treating” or “therapeutic intervention” relates to the management and care of a subject for the purpose of combating a condition such as a disease or disorder. The term is intended to include the full spectrum of treatments for a given condition from which the subject is suffering, such as administration of the therapeutically effective compound to alleviate the symptoms or complications, to delay the progression of the disease, disorder or condition, to alleviate or relief the symptoms and complications, and/or to cure or eliminate the disease, disorder or condition as well as to prevent the condition, wherein prevention is to be understood as the management and care of an individual for the purpose of combating the disease, condition or disorder and includes the administration of the active compounds to prevent the onset of the symptoms or complications.

The term “therapeutic treatment” relates to any treatment which improves the health status and/or prolongs (increases) the lifespan of an individual. Said treatment may eliminate the disease in an individual, arrest or slow the development of a disease in an individual, inhibit or slow the development of a disease in an individual, decrease the frequency or severity of symptoms in an individual, and/or decrease the recurrence in an individual who currently has or who previously has had a disease.

The terms “prophylactic treatment” or “preventive treatment” relate to any treatment that is intended to prevent a disease from occurring in an individual. The terms “prophylactic treatment” or “preventive treatment” are used herein interchangeably.

The terms “individual” and “subject” are used herein interchangeably. They refer to a human or another mammal (e.g. mouse, rat, rabbit, dog, cat, cattle, swine, sheep, horse or primate) that can be afflicted with or is susceptible to a disease or disorder (e.g., cancer) but may or may not have the disease or disorder. In many embodiments, the individual is a human being. Unless otherwise stated, the terms “individual” and “subject” do not denote a particular age, and thus encompass adults, elderlies, children, and newborns. In embodiments of the present disclosure, the “individual” or “subject” is a “patient”.

The term “patient” means an individual or subject for treatment, in particular a diseased individual or subject.

In one embodiment of the disclosure, the aim is to provide an immune response against diseased cells expressing an antigen such as cancer cells expressing a tumor antigen, and to treat a disease such as a cancer disease involving cells expressing an antigen such as a tumor antigen.

An immune response against an antigen may be elicited which may be therapeutic or partially or fully protective. Pharmaceutical compositions described herein are applicable for inducing or enhancing an immune response. Pharmaceutical compositions described herein are thus useful in a prophylactic and/or therapeutic treatment of a disease involving an antigen.

As used herein, “immune response” refers to an integrated bodily response to an antigen or a cell expressing an antigen and refers to a cellular immune response and/or a humoral immune response.

“Cell-mediated immunity”, “cellular immunity”, “cellular immune response”, or similar terms are meant to include a cellular response directed to cells characterized by expression of an antigen, in particular characterized by presentation of an antigen with class I or class II MHC. The cellular response relates to cells called T cells or T lymphocytes which act as either “helpers” or “killers”. The helper T cells (also termed CD4⁺ T cells) play a central role by regulating the immune response and the killer cells (also termed cytotoxic T cells, cytolytic T cells, CD8⁺ T cells or CTLs) kill diseased cells such as cancer cells, preventing the production of more diseased cells.

The present disclosure contemplates an immune response that may be protective, preventive, prophylactic and/or therapeutic. As used herein, “induces [or inducing] an immune response” may indicate that no immune response against a particular antigen was present before induction or it may indicate that there was a basal level of immune response against a particular antigen before induction, which was enhanced after induction. Therefore, “induces [or inducing] an immune response” includes “enhances [or enhancing] an immune response”.

The term “immunotherapy” relates to the treatment of a disease or condition by inducing, or enhancing an immune response. The term “immunotherapy” includes antigen immunization or antigen vaccination.

The terms “immunization” or “vaccination” describe the process of administering an antigen to an individual with the purpose of inducing an immune response, for example, for therapeutic or prophylactic reasons.

The term “macrophage” refers to a subgroup of phagocytic cells produced by the differentiation of monocytes. Macrophages which are activated by inflammation, immune cytokines or microbial products nonspecifically engulf and kill foreign pathogens within the macrophage by hydrolytic and oxidative attack resulting in degradation of the pathogen. Peptides from degraded proteins are displayed on the macrophage cell surface where they can be recognized by T cells, and they can directly interact with antibodies on the B cell surface, resulting in T and B cell activation and further stimulation of the immune response. Macrophages belong to the class of antigen presenting cells. In one embodiment, the macrophages are splenic macrophages.

The term “dendritic cell” (DC) refers to another subtype of phagocytic cells belonging to the class of antigen presenting cells. In one embodiment, dendritic cells are derived from hematopoietic bone marrow progenitor cells. These progenitor cells initially transform into immature dendritic cells. These immature cells are characterized by high phagocytic activity and low T cell activation potential. Immature dendritic cells constantly sample the surrounding environment for pathogens such as viruses and bacteria. Once they have come into contact with a presentable antigen, they become activated into mature dendritic cells and begin to migrate to the spleen or to the lymph node. Immature dendritic cells phagocytose pathogens and degrade their proteins into small pieces and upon maturation present those fragments at their cell surface using MHC molecules. Simultaneously, they upregulate cell-surface receptors that act as co-receptors in T cell activation such as CD80, CD86, and CD40 greatly enhancing their ability to activate T cells. They also upregulate CCR7, a chemotactic receptor that induces the dendritic cell to travel through the blood stream to the spleen or through the lymphatic system to a lymph node. Here they act as antigen-presenting cells and activate helper T cells and killer T cells as well as B cells by presenting them antigens, alongside non-antigen specific co-stimulatory signals. Thus, dendritic cells can actively induce a T cell- or B cell-related immune response. In one embodiment, the dendritic cells are splenic dendritic cells.

The term “antigen presenting cell” (APC) is a cell of a variety of cells capable of displaying, acquiring, and/or presenting at least one antigen or antigenic fragment on (or at) its cell surface. Antigen-presenting cells can be distinguished in professional antigen presenting cells and non-professional antigen presenting cells.

The term “professional antigen presenting cells” relates to antigen presenting cells which constitutively express the Major Histocompatibility Complex class II (MHC class II) molecules required for interaction with naive T cells. If a T cell interacts with the MHC class II molecule complex on the membrane of the antigen presenting cell, the antigen presenting cell produces a co-stimulatory molecule inducing activation of the T cell. Professional antigen presenting cells comprise dendritic cells and macrophages.

The term “non-professional antigen presenting cells” relates to antigen presenting cells which do not constitutively express MHC class II molecules, but upon stimulation by certain cytokines such as interferon-gamma. Exemplary, non-professional antigen presenting cells include fibroblasts, thymic epithelial cells, thyroid epithelial cells, glial cells, pancreatic beta cells or vascular endothelial cells.

“Antigen processing” refers to the degradation of an antigen into procession products, which are fragments of said antigen (e.g., the degradation of a protein into peptides) and the association of one or more of these fragments (e.g., via binding) with MHC molecules for presentation by cells, such as antigen presenting cells to specific T cells.

The term “disease involving an antigen”, “disease involving cells expressing an antigen” or similar terms refer to any disease which implicates an antigen, e.g. a disease which is characterized by the presence of an antigen. The disease involving an antigen can be an infectious disease, or a cancer disease or simply cancer. As mentioned above, the antigen may be a disease-associated antigen, such as a tumor-associated antigen, a viral antigen, or a bacterial antigen. In one embodiment, a disease involving an antigen is a disease involving cells expressing an antigen, preferably on the cell surface.

The term “infectious disease” refers to any disease which can be transmitted from individual to individual or from organism to organism, and is caused by a microbial agent (e.g. common cold). Infectious diseases are known in the art and include, for example, a viral disease, a bacterial disease, or a parasitic disease, which diseases are caused by a virus, a bacterium, and a parasite, respectively. In this regard, the infectious disease can be, for example, hepatitis, sexually transmitted diseases (e.g. chlamydia or gonorrhea), tuberculosis, HIV/acquired immune deficiency syndrome (AIDS), diphtheria, hepatitis B, hepatitis C, cholera, severe acute respiratory syndrome (SARS), the bird flu, and influenza.

The terms “cancer disease” or “cancer” refer to or describe the physiological condition in an individual that is typically characterized by unregulated cell growth. Examples of cancers include, but are not limited to, carcinoma, lymphoma, blastoma, sarcoma, and leukemia. More particularly, examples of such cancers include bone cancer, blood cancer, lung cancer, liver cancer, pancreatic cancer, skin cancer, cancer of the head or neck, cutaneous or intraocular melanoma, uterine cancer, ovarian cancer, rectal cancer, cancer of the anal region, stomach cancer, colon cancer, breast cancer, prostate cancer, uterine cancer, carcinoma of the sexual and reproductive organs, Hodgkin's Disease, cancer of the esophagus, cancer of the small intestine, cancer of the endocrine system, cancer of the thyroid gland, cancer of the parathyroid gland, cancer of the adrenal gland, sarcoma of soft tissue, cancer of the bladder, cancer of the kidney, renal cell carcinoma, carcinoma of the renal pelvis, neoplasms of the central nervous system (CNS), neuroectodermal cancer, spinal axis tumors, glioma, meningioma, and pituitary adenoma. The term “cancer” according to the disclosure also comprises cancer metastases.

The term “solid tumor” or “solid cancer” as used herein refers to the manifestation of a cancerous mass, as is well known in the art for example in Harrison's Principles of Internal Medicine, 14th edition.

Preferably, the term refers to a cancer or carcinoma of body tissues other than blood, preferably other than blood, bone marrow, and lymphoid system. For example, but not by way of limitation, solid tumors include cancers of the prostate, lung cancer, colorectal tissue, bladder, oropharyngeal/laryngeal tissue, kidney, breast, endometrium, ovary, cervix, stomach, pancrease, brain, and central nervous system.

Combination strategies in cancer treatment may be desirable due to a resulting synergistic effect, which may be considerably stronger than the impact of a monotherapeutic approach. In one embodiment, the pharmaceutical composition is administered with an immunotherapeutic agent. As used herein “immunotherapeutic agent” relates to any agent that may be involved in activating a specific immune response and/or immune effector function(s). The present disclosure contemplates the use of an antibody as an immunotherapeutic agent. Without wishing to be bound by theory, antibodies are capable of achieving a therapeutic effect against cancer cells through various mechanisms, including inducing apoptosis, block components of signal transduction pathways or inhibiting proliferation of tumor cells. In certain embodiments, the antibody is a monoclonal antibody. A monoclonal antibody may induce cell death via antibody-dependent cell mediated cytotoxicity (ADCC), or bind complement proteins, leading to direct cell toxicity, known as complement dependent cytotoxicity (CDC). Non-limiting examples of anti-cancer antibodies and potential antibody targets (in brackets) which may be used in combination with the present disclosure include: Abagovomab (CA-125), Abciximab (CD41), Adecatumumab (EpCAM), Afutuzumab (CD20), Alacizumab pegol (VEGFR2), Altumomab pentetate (CEA), Amatuximab (MORAb-009), Anatumomab mafenatox (TAG-72), Apolizumab (HLA-DR), Arcitumomab (CEA), Atezolizumab (PD-L1), Bavituximab (phosphatidylserine), Bectumomab (CD22), Belimumab (BAFF), Bevacizumab (VEGF-A), Bivatuzumab mertansine (CD44 v6), Blinatumomab (CD 19), Brentuximab vedotin (CD30 TNFRSF8), Cantuzumab mertansin (mucin CanAg), Cantuzumab ravtansine (MUC1), Capromab pendetide (prostatic carcinoma cells), Carlumab (CNT0888), Catumaxomab (EpCAM, CD3), Cetuximab (EGFR), Citatuzumab bogatox (EpCAM), Cixutumumab (IGF-1 receptor), Claudiximab (Claudin), Clivatuzumab tetraxetan (MUC1), Conatumumab (TRAIL-R2), Dacetuzumab (CD40), Dalotuzumab (insulin-like growth factor I receptor), Denosumab (RANKL), Detumomab (B-lymphoma cell), Drozitumab (DR5), Ecromeximab (GD3 ganglioside), Edrecolomab (EpCAM), Elotuzumab (SLAMF7), Enavatuzumab (PDL192), Ensituximab (NPC-1C), Epratuzumab (CD22), Ertumaxomab (HER2/neu, CD3), Etaracizumab (integrin αvβ3), Farletuzumab (folate receptor 1), FBTA05 (CD20), Ficlatuzumab (SCH 900105), Figitumumab (IGF-1 receptor), Flanvotumab (glycoprotein 75), Fresolimumab (TGF-β), Galiximab (CD80), Ganitumab (IGF-I), Gemtuzumab ozogamicin (CD33), Gevokizumab (ILIβ), Girentuximab (carbonic anhydrase 9 (CA-IX)), Glembatumumab vedotin (GPNMB), Ibritumomab tiuxetan (CD20), Icrucumab (VEGFR-1), Igovoma (CA-125), Indatuximab ravtansine (SDC1), Intetumumab (CD51), Inotuzumab ozogamicin (CD22), Ipilimumab (CD 152), Iratumumab (CD30), Labetuzumab (CEA), Lexatumumab (TRAIL-R2), Libivirumab (hepatitis B surface antigen), Lintuzumab (CD33), Lorvotuzumab mertansine (CD56), Lucatumumab (CD40), Lumiliximab (CD23), Mapatumumab (TRAIL-R1), Matuzumab (EGFR), Mepolizumab (IL5), Milatuzumab (CD74), Mitumomab (GD3 ganglioside), Mogamulizumab (CCR4), Moxetumomab pasudotox (CD22), Nacolomab tafenatox (C242 antigen), Naptumomab estafenatox (5T4), Namatumab (RON), Necitumumab (EGFR), Nimotuzumab (EGFR), Nivolumab (IgG4), Ofatumumab (CD20), Olaratumab (PDGF-R a), Onartuzumab (human scatter factor receptor kinase), Oportuzumab monatox (EpCAM), Oregovomab (CA-125), Oxelumab (OX-40), Panitumumab (EGFR), Patritumab (HER3), Pemtumoma (MUC1), Pertuzuma (HER2/neu), Pintumomab (adenocarcinoma antigen), Pritumumab (vimentin), Racotumomab (N-glycolylneuraminic acid), Radretumab (fibronectin extra domain-B), Rafivirumab (rabies virus glycoprotein), Ramucirumab (VEGFR2), Rilotumumab (HGF), Rituximab (CD20), Robatumumab (IGF-1 receptor), Samalizumab (CD200), Sibrotuzumab (FAP), Siltuximab (IL6), Tabalumab (BAFF), Tacatuzumab tetraxetan (alpha-fetoprotein), Taplitumomab paptox (CD 19), Tenatumomab (tenascin C), Teprotumumab (CD221), Ticilimumab (CTLA-4), Tigatuzumab (TRAIL-R2), TNX-650 (IL13), Tositumomab (CD20), Trastuzumab (HER2/neu), TRBS07 (GD2), Tremelimumab (CTLA-4), Tucotuzumab celmoleukin (EpCAM), Ublituximab (MS4A1), Urelumab (4-1 BB), Volociximab (integrin α5β1), Votumumab (tumor antigen CTAA 16.88), Zalutumumab (EGFR), and Zanolimumab (CD4).

Citation of documents and studies referenced herein is not intended as an admission that any of the foregoing is pertinent prior art. All statements as to the contents of these documents are based on the information available to the applicants and do not constitute any admission as to the correctness of the contents of these documents.

The following description is presented to enable a person of ordinary skill in the art to make and use the various embodiments. Descriptions of specific devices, techniques, and applications are provided only as examples. Various modifications to the examples described herein will be readily apparent to those of ordinary skill in the art, and the general principles defined herein may be applied to other examples and applications without departing from the spirit and scope of the various embodiments. Thus, the various embodiments are not intended to be limited to the examples described herein and shown, but are to be accorded the scope consistent with the claims.

### EXAMPLES

### Example 1: CD8-DARPin Identification

### Ribosome Display

Ribosome display selection was carried out as described by Hartmann and colleagues (Hartmann et al. 2018). Briefly, for the first three selection rounds, the translated VV-N3C DARPin library was subjected to pre-panning steps with immobilized neutravidin or streptavidin (both 20 μM). For on-target selection, the library was incubated with immobilized hCD8αβ-Fc (50 nm). The resulting DARPin library was amplified and used as template for the next selection round. After three rounds with immobilized target protein, the fourth round of selection was carried out with target in solution. Prior to on-target selection, a pre-selection step using 0.9 pmol unbiotinylated hCD8αα-Fc was performed. Then, the library was exposed to biotinylated hCD8αβFc target protein (0.65 μmol) and unbiotinylated hCD8ααFc in 100-fold molar excess (65 pmol). Selection round five was again performed using immobilized proteins and included a pre-selection step with immobilized CD8αα-Fc (20 nM) prior to incubation with hCD8αβ-Fc. Finally, a sixth round of selection was carried out in solution as follows: Pre-incubation with 0.9 pmol soluble CD8αα-Fc was followed by a combined off-rate and counter-selection step, where the library was co-incubated with biotinylated target protein (0.65 pmol), an excess of unbiotinylated hCD8αα-Fc and unbiotinylated hCD8αβ-Fc (both 65 pmol). After the fifth and sixth selection round, DARPin-encoding DNA fragments were analyzed for CD8 binding by single clone analysis.

### DARPin Expression and Crude Lysate Preparation

To test selected DARPins for specific binding to CD8 after the fifth and sixth selection round, DNA fragments were cloned into the bacterial expression vector pQE-HisHA and transformed into E. coli XL1-blue as described before (Hartmann et al. 2018). Single clones were picked and cultured overnight in 600 μl 2YT medium (2YT, 1% glucose, 100 μg/ml ampicillin) at 37° C. before cultures were diluted to an OD₆₀₀ of 0.1 and expression of the DARPins was induced by addition of 100 mL of 5.5 mM isopropyl-b-D-thiogalactopyranosid (IPTG) in 2YT medium. After culture for 5 h at 37° C., bacteria were harvested by centrifugation and pellets were stored overnight at −80° C. The next day, pellets were thawed on ice and lysed by addition of B_PER solution and subsequent incubation at room temperature for 2 hours. The lysate was pelleted to remove cell debris, the supernatant containing crude DARPin was aliquoted and stored at −80° C. until use in cellular binding assays. Protein content was determined by Bradford assay. Crude lysate preparations were always handled on ice and subjected to a maximum of three freeze-thaw cycles to avoid loss of protein quality. DARPin clones were sequenced using standard sequencing technologies to obtain DNA and protein sequences.

### Cellular Binding Assays

To analyze specific binding of DARPins to human and NHP CD8, cellular binding assays using Molt4.8 and J76S8ab cells as well as primary human and NHP PBMC (isolated and activated as described above) were carried out. In brief, 1×10⁵ cells were washed once with wash buffer (PBS, 2% FCS, 0.1% NaN₃) and incubated with 10 μl of crude DARPin extracts in a total volume of 200 μl for 60 min at 4° C. Following incubation, cells were washed twice with wash buffer, stained with fluorescently labelled antibodies and analyzed by flow cytometry as described below. Screening for CD8 binding by cellular binding assays using cell lines and validation by binding to human and NHP PBMC was carried out at n=1.

### Example 2: CD8-Specific Binding of DARPins

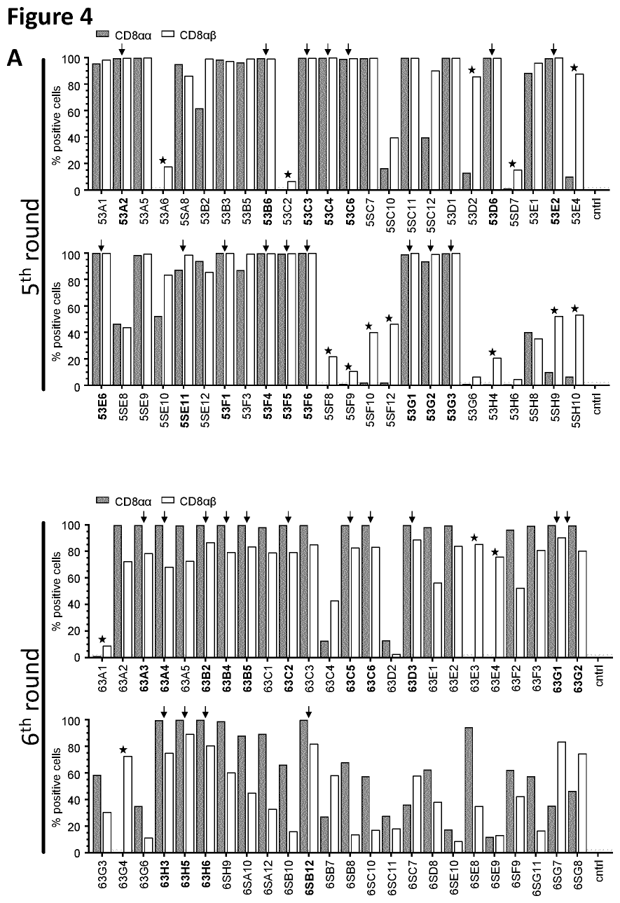

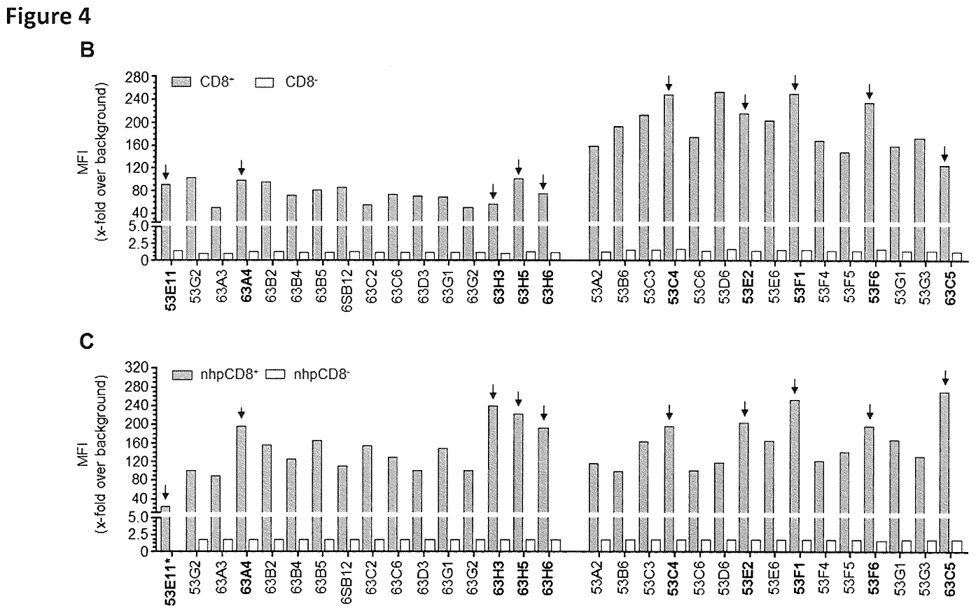

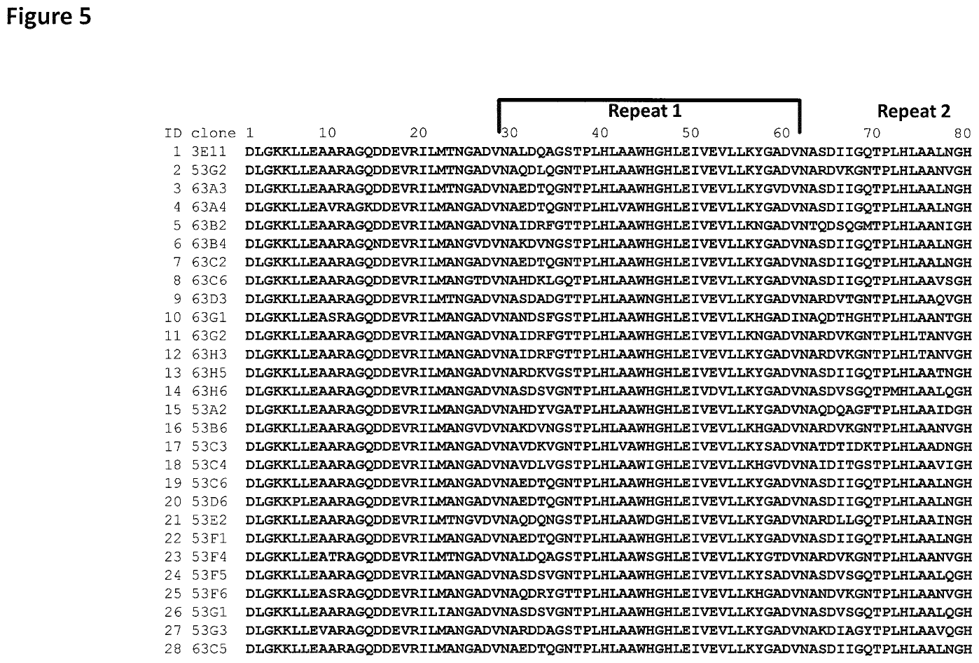

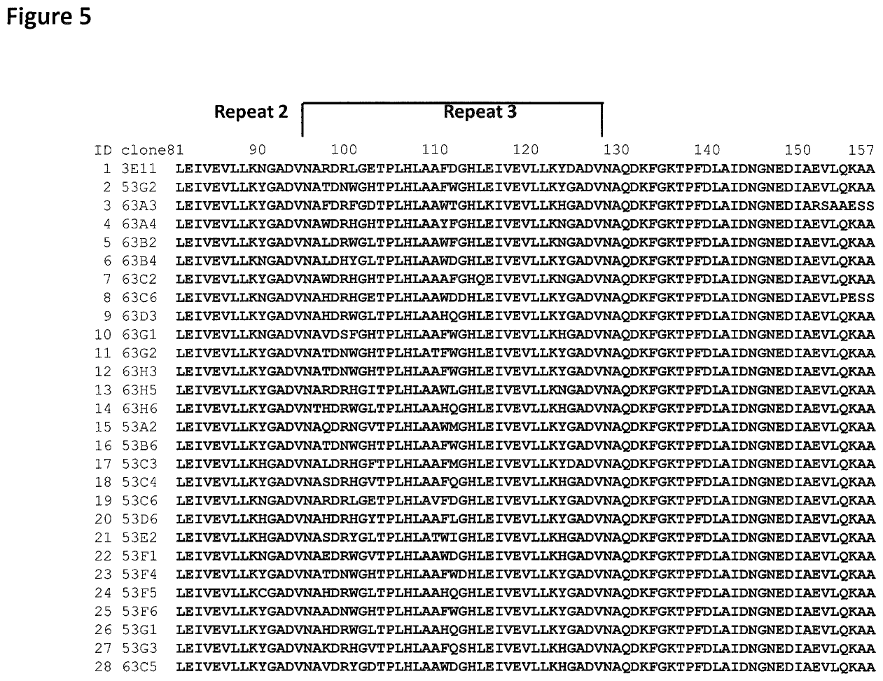

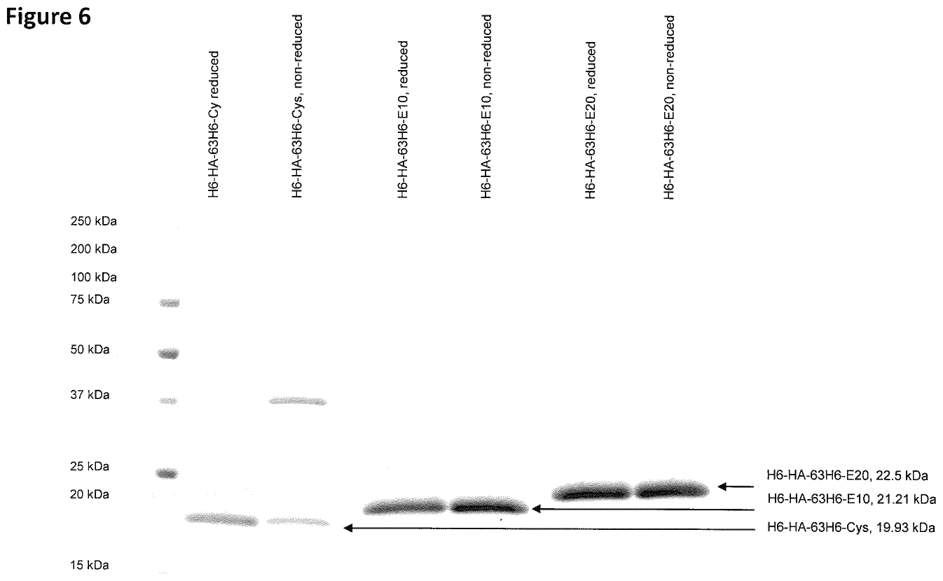

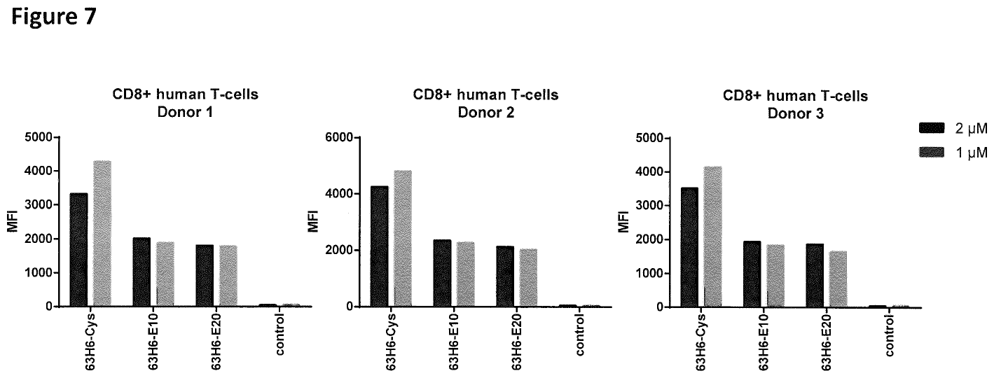

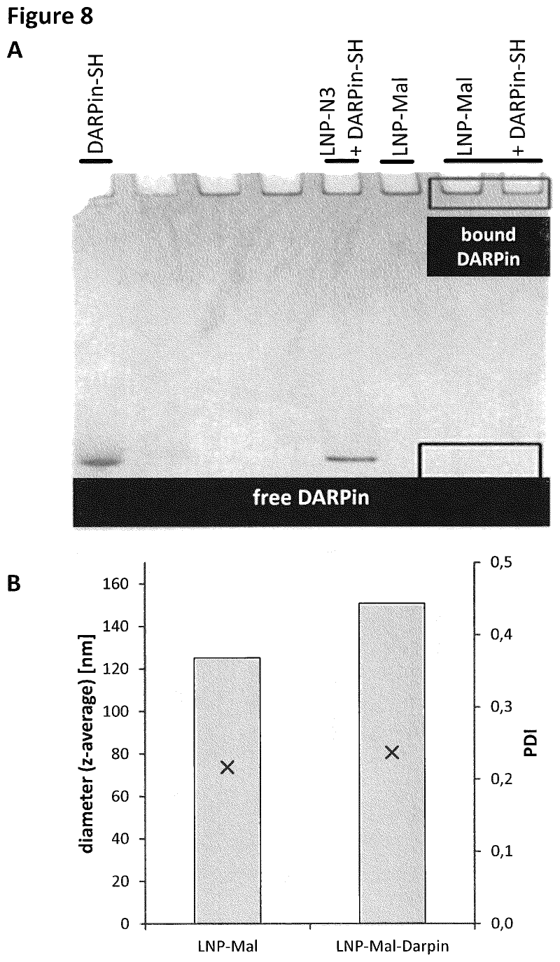

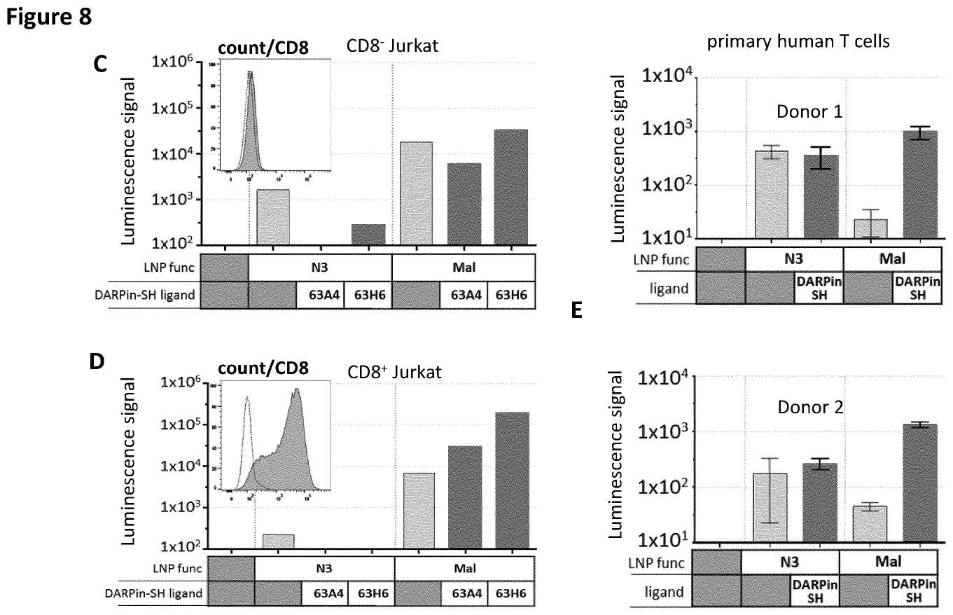

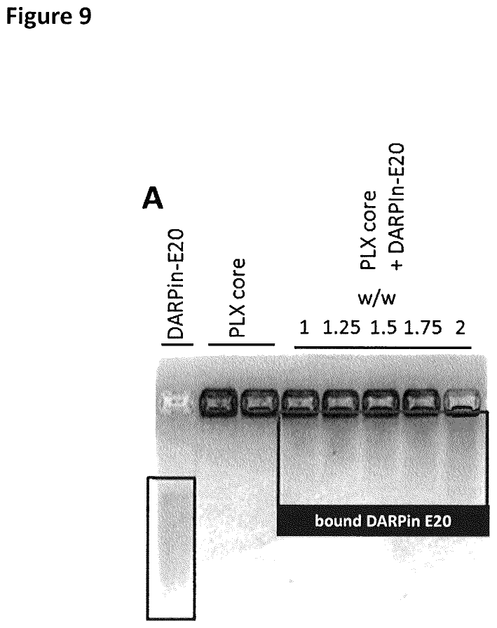

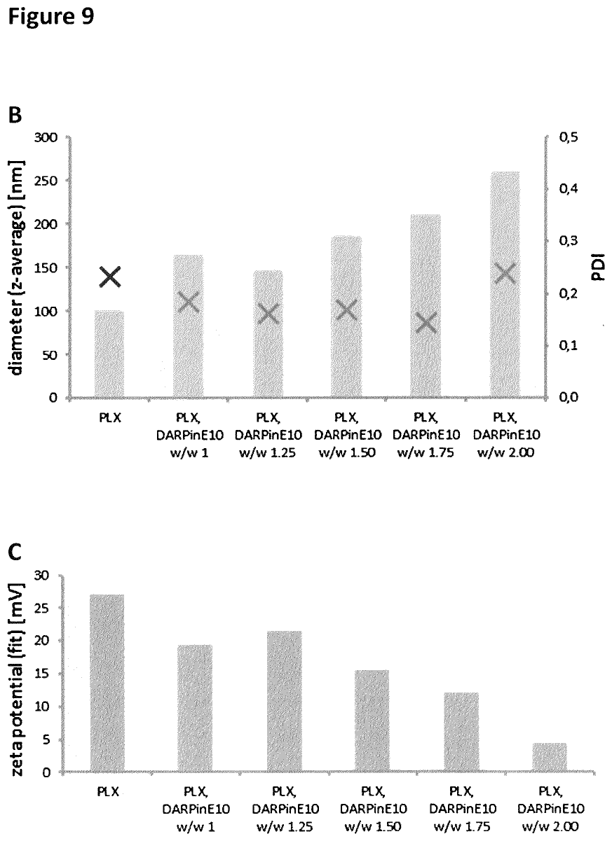

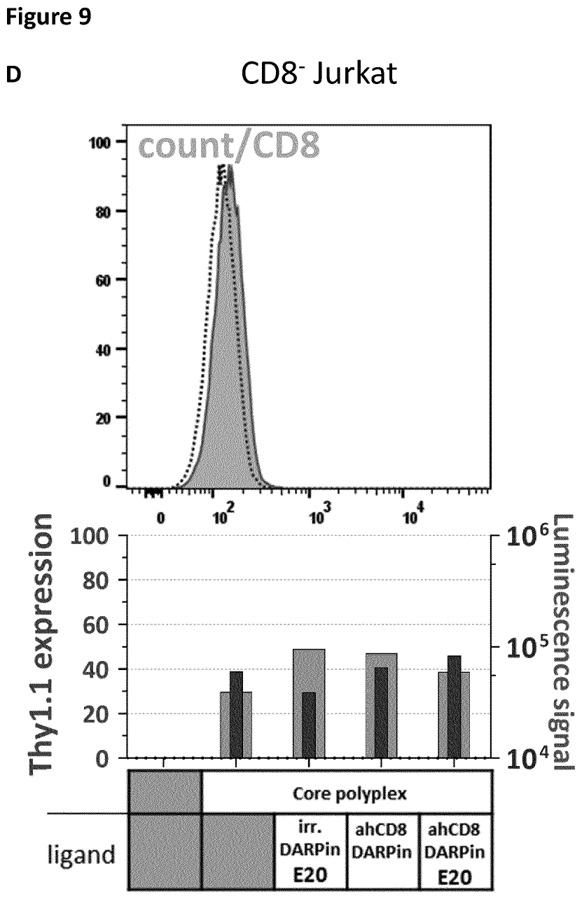

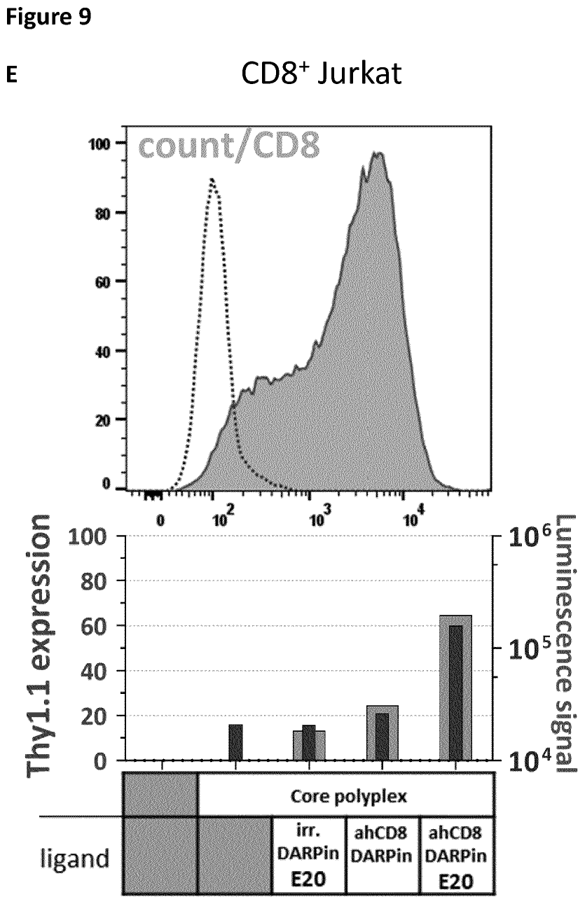

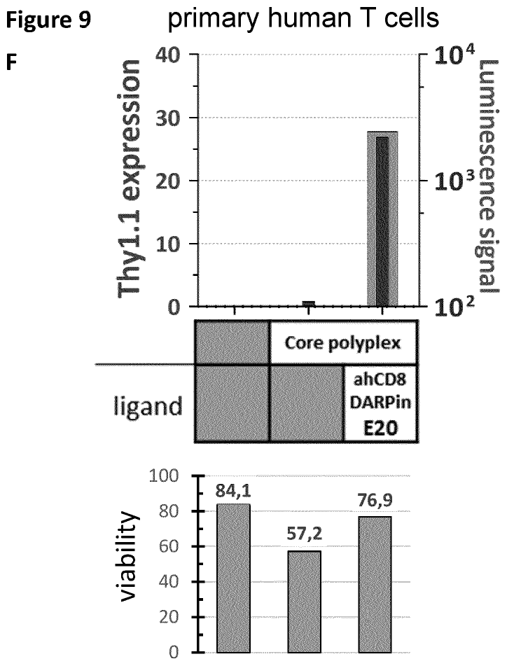

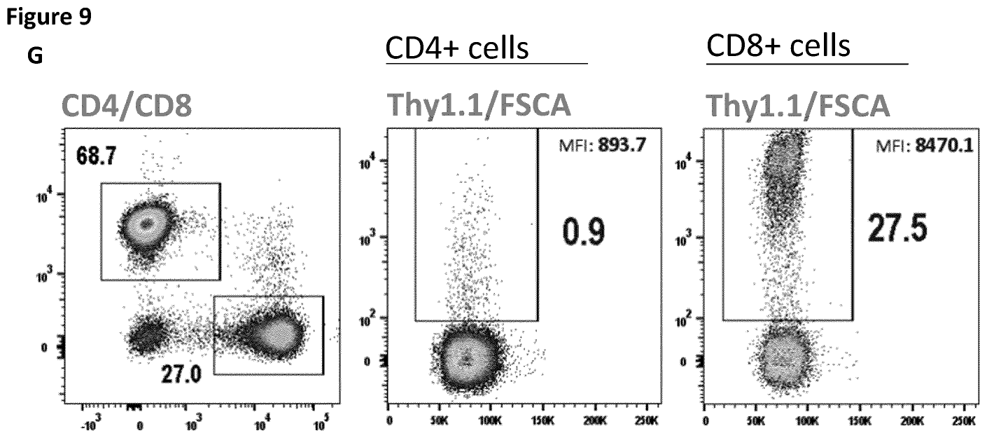

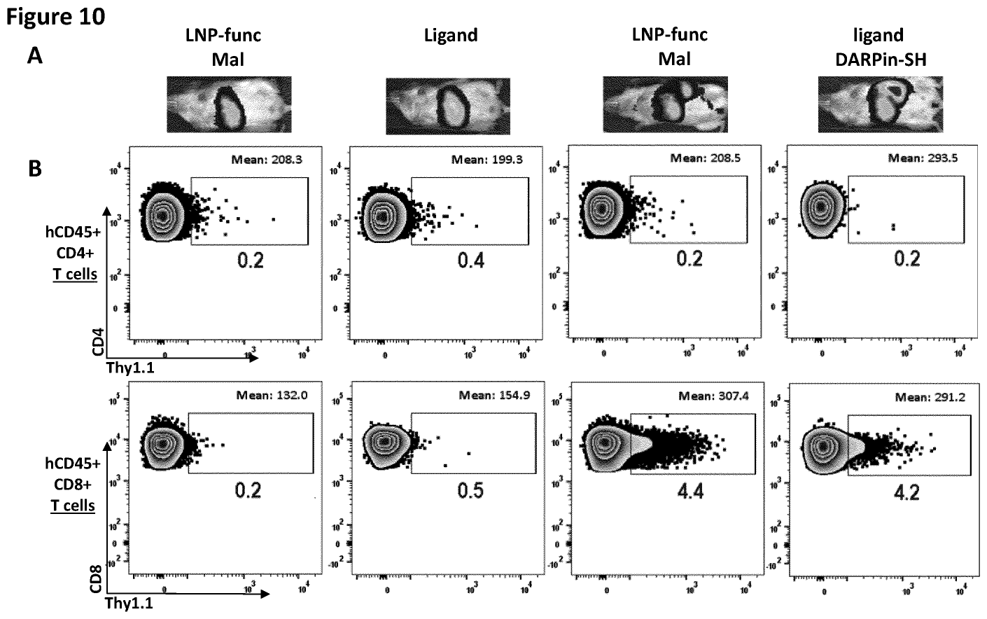

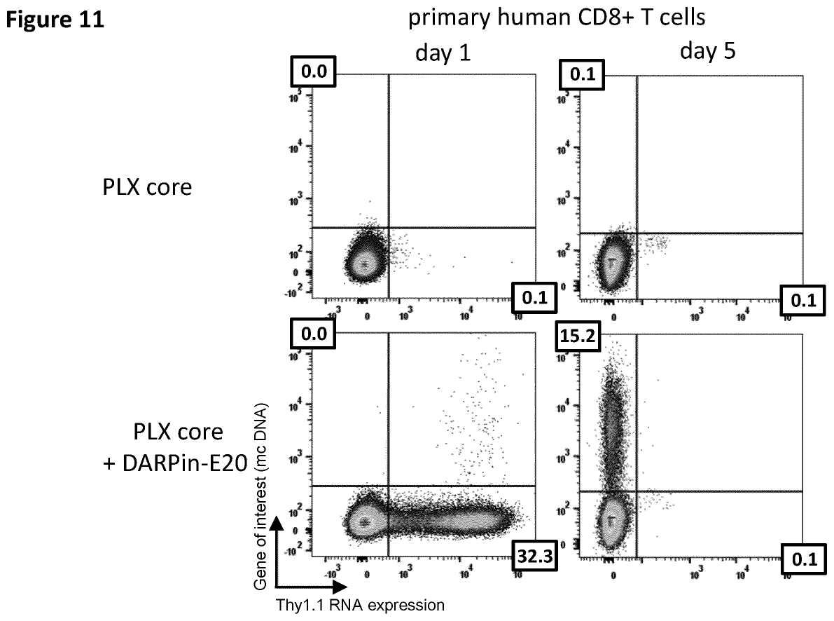

The previously described combinatorial DARPin library VV-N3C (Hartmann et al. 2018) was screened for CD8-specific DARPins by ribosome display using the generated recombinant CD8 proteins as bait. In total, up to six selection rounds were carried out. The first three as well as the fifth round were performed with immobilized CD8αβ-Fc as bait protein including pre-selection steps against neutravidin, streptavidin and immobilized Fc protein to exclude selection of DARPins with unwanted specificity. Fourth and sixth round were carried out in solution and included a counter selection with unbiotinlyated CD8αβ-Fc and CD8αα-Fc to select binders with high affinity for CD8αβ. To identify the best CD8-specific DARPins for targeted gene transfer, the output repertoire was screened in a two-step procedure. First, CD8 binding was evaluated in cell based assays. In the second step, the gene transfer activity of 10 clones was assessed. In total, 94 DARPin clones obtained from the fifth and sixth selection round were expressed in E. coli and tested for binding to Molt4.8 cells (express CD8αα) and to J67S8ab cells (express CD84). Of these, 31 individual DARPin clones were selected that bound both cell lines equally well (FIG. 4A) and were further analyzed for binding to primary human and NHP T cells. All candidates specifically bound to human CD8⁺ cells, while CD8⁻ cells were not decorated above background (FIG. 4B). Notably, variations in the cell staining intensity across the DARPin candidates were observed (FIG. 4B). In the next step, cross-reactivity of these DAPRins to macaque PBMC, were assessed. All the identified human CD8-specific DARPins also bound to CD8 on NHP PBMC (FIG. 4C). Of the identified CD8 binders, five DARPins binding human CD8 with an intermediate MFI and five candidates binding with a high MFI were selected for further characterization. 28 of the candidates have been successfully characterized and sequencing revealed that each of these candidates (Tab. 1) had a unique amino acid sequence.

TABLE 1
Amino acid sequences of designed
ankyrin repeat proteins (DARPins)

| SEQ ID | Clone | Sequence |
|---|---|---|
| 1 | 53E11 | DLGKKLLEAARAGQDDEVRILMTNG |
| | | ADVNALDQAGSTPLHLAAWHGHLEI |
| | | VEVLLKYGADVNASDIIGQTPLHLA |
| | | ALNGHLEIVEVLLKNGADVN |
| | | ARDRLGETPLHLAAFDGHLEIVEVL |
| | | LKYDADVNAQDKFGKTPFDLAIDNG |
| | | NEDIAEVLQKAA |
| 2 | 53G2 | DLGKKLLEAARAGQDDEVRILMTNG |
| | | ADVNAQDLQGNTPLHLAAWHGHLEI |
| | | VEVLLKYGADVNARDVKGNTPLHLA |
| | | ANVGHLEIVEVLLKYGAD |
| | | VNATDNWGHTPLHLAAFWGHLEIVE |
| | | VLLKYGADVNAQDKFGKTPFDLAID |
| | | NGNEDIAEVLQKAA |
| 3 | 63A3 | DLGKKLLEAARAGQDDEVRILMTNG |
| | | ADVNAEDTQGNTPLHLAAWHGHLEI |
| | | VEVLLKYGVDVNASDIIGQTPLHLA |
| | | ALNGHLEIVEVLLKYGADVN |
| | | AFDRFGDTPLHLAAWTGHLKIVEVL |
| | | LKHGADVNAQDKFGKTPFDLAIDNG |
| | | NEDIARSAAESS |
| 4 | 63A4 | DLGKKLLEAVRAGKDDEVRILMANG |
| | | ADVNAEDTQGNTPLHLVAWHGHLEI |
| | | VEVLLKYGADVNASDIIGQTPLHLA |
| | | ALNGHLEIVEVLLKYGADVN |
| | | AWDRHGHTPLHLAAYFGHLEIVEVL |
| | | LKNGADVNAQDKFGKTPFDLAIDNG |
| | | NEDIAEVLQKAA |
| 5 | 63B2 | DLGKKLLEAARAGQDDEVRILMANG |
| | | ADVNAIDRFGTTPLHLAAWHGHLEI |
| | | VEVLLKNGADVNTQDSQGMTPLHLA |
| | | ANIGHLEIVEVLLKYGADV |
| | | NALDRWGLTPLHLAAWFGHLEIVEV |
| | | LLKNGADVNAQDKFGKTPFDLAIDN |
| | | GNEDIAEVLQKAA |
| 6 | 63B4 | DLGKKLLEAARAGQNDEVRILMANG |
| | | VDVNAKDVNGSTPLHLAAWHGHLEI |
| | | VEVLLKYGADVNASDIIGQTPLHLA |
| | | ALNGHLEIVEVLLKNGADVN |
| | | ALDHYGLTPLHLAAWDGHLEIVEVL |
| | | LKYGADVNAQDKFGKTPFDLAIDNG |
| | | NEDIAEVLQKAA |
| 7 | 63C2 | DLGKKLLEAARAGQDDEVRILMANG |
| | | ADVNAEDTQGNTPLHLAAWHGHLEI |
| | | VEVLLKYGADVNASDIIGQTPLHLA |
| | | ALNGHLEIVEVLLKYGADVN |
| | | AWDRHGHTPLHLAAAFGHQEIVEVL |
| | | LKNGADVNAQDKFGKTPFDLAIDNG |
| | | NEDIAEVLQKAA |
| 8 | 63C6 | DLGKKLLEAARAGQDDEVRILMANG |
| | | TDVNAHDKLGQTPLHLAAWHGHLEI |
| | | VEVLLKYGADVNASDIIGQTPLHLA |
| | | AVSGHLEIVEVLLKNGADVN |
| | | AHDRHGETPLHLAAWDDHLEIVEVL |
| | | LKYGADVNAQDKFGKTPFDLAIDNG |
| | | NEDIAEVLPESS |
| 9 | 63D3 | DLGKKLLEAARAGQDDEVRILMTNG |
| | | ADVNASDADGTTPLHLAAWNGHLEI |
| | | VEVLLKYGADVNARDVTGNTPLHLA |
| | | AQVGHLEIVEVLLKYGADV |
| | | NAHDRWGLTPLHLAAHQGHLEIVEV |
| | | LLKYGADVNAQDKFGKTPFDLAIDN |
| | | GNEDIAEVLQKAA |
| 10 | 63G1 | DLGKKLLEASRAGQDDEVRILMANG |
| | | ADVNANDSFGSTPLHLAAWHGHLEI |
| | | VEVLLKHGADINAQDTHGHTPLHLA |
| | | ANTGHLEIVEVLLKNGADV |
| | | NAVDSFGHTPLHLAAFWGHLEIVEV |
| | | LLKHGADVNAQDKFGKTPFDLAIDN |
| | | GNEDIAEVLQKAA |
| 11 | 63G2 | DLGKKLLEAARAGQDDEVRILMANG |
| | | ADVNAIDRFGTTPLHLAAWHGHLEI |
| | | VEVLLKNGADVNARDVKGNTPLHLT |
| | | ANVGHLEIVEVLLKYGADV |
| | | NATDNWGHTPLHLATFWGHLEIVEV |
| | | LLKYGADVNAQDKFGKTPFDLAIDN |
| | | GNEDIAEVLQKAA |
| 12 | 63H3 | DLGKKLLEAARAGQDDEVRILMANG |
| | | ADVNAIDRFGTTPLHLAAWHGHLEI |
| | | VEVLLKYGADVNARDVKGNTPLHLT |
| | | ANVGHLEIVEVLLKYGADV |
| | | NATDNWGHTPLHLAAFWGHLEIVEV |
| | | LLKYGADVNAQDKFGKTPFDLAIDN |
| | | GNEDIAEVLQKAA |
| 13 | 63H5 | DLGKKLLEAARAGQDDEVRILMANG |
| | | ADVNARDKVGSTPLHLAAWHGHLEI |
| | | VEVLLKYGADVNASDIIGQTPLHLA |
| | | ATNGHLEIVEVLLKYGADVN |
| | | ARDRHGITPLHLAAWLGHLEIVEVL |
| | | LKNGADVNAQDKFGKTPFDLAIDNG |
| | | NEDIAEVLQKAA |
| 14 | 63H6 | DLGKKLLEAARAGQDDEVRILMANG |
| | | ADVNASDSVGNTPLHLAAWHGHLEI |
| | | VDVLLKYGADVNASDVSGQTPMHLA |
| | | ALQGHLEIVEVLLKYGAD |
| | | VNTHDRWGLTPLHLAAHQGHLEIVE |
| | | VLLKHGADVNAQDKFGKTPFDLAID |
| | | NGNEDIAEVLQKAA |
| 15 | 53A2 | DLGKKLLEAARAGQDDEVRILMANG |
| | | ADVNAHDYVGATPLHLAAWHGHLEI |
| | | VEVLLKYGADVNAQDQAGFTPLHLA |
| | | AIDGHLEIVEVLLKYGADV |
| | | NAQDRNGVTPLHLAAWMGHLEIVEV |
| | | LLKYGADVNAQDKFGKTPFDLAIDN |
| | | GNEDIAEVLQKAA |
| 16 | 53B6 | DLGKKLLEAARAGQDDEVRILMANG |
| | | VDVNAKDVNGSTPLHLAAWHGHLEI |
| | | VEVLLKHGADVNARDVKGNTPLHLA |
| | | ANVGHLEIVEVLLKYGAD |
| | | VNATDNWGHTPLHLAAFWGHLEIVE |
| | | VLLKYGADVNAQDKFGKTPFDLAID |
| | | NGNEDIAEVLQKAA |
| 17 | 53C3 | DLGKKLLEAARAGQDDEVRILMANG |
| | | ADVNAVDKVGNTPLHLVAWHGHLEI |
| | | VEVLLKYSADVNATDTIDKTPLHLA |
| | | ADNGHLEIVEVLLKHGADV |
| | | NALDRHGFTPLHLAAFMGHLEIVEV |
| | | LLKYDADVNAQDKFGKTPFDLAIDN |
| | | GNEDIAEVLQKAA |
| 18 | 5304 | DLGKKLLEAARAGQDDEVRILMANG |
| | | ADVNAVDLVGSTPLHLAAWIGHLEI |
| | | VEVLLKHGVDVNAIDITGSTPLHLA |
| | | AVIGHLEIVEVLLKYGADVNA |
| | | SDRHGVTPLHLAAFQGHLEIVEVLL |
| | | KHGADVNAQDKFGKTPFDLAIDNGN |
| | | EDIAEVLQKAA |
| 19 | 53C6 | DLGKKLLEAARAGQDDEVRILMANG |
| | | ADVNAEDTQGNTPLHLAAWHGHLEI |
| | | VEVLLKYGADVNASDIIGQTPLHLA |
| | | ALNGHLEIVEVLLKNGADV |
| | | NARDRLGETPLHLAVFDGHLEIVEV |
| | | LLKYGADVNAQDKFGKTPFDLAIDN |
| | | GNEDIAEVLQKAA |
| 20 | 53D6 | DLGKKPLEAARAGQDDEVRILMANG |
| | | ADVNAEDTQGNTPLHLAAWHGHLEI |
| | | VEVLLKYGADVNASDIIGQTPLHLA |
| | | ALNGHLEIVEVLLKHGADV |
| | | NAHDRHGYTPLHLAAFLGHLEIVEV |
| | | LLKYGADVNAQDKFGKTPFDLAIDN |
| | | GNEDIAEVLQKAA |
| 21 | 53E2 | DLGKKLLEAARAGQDDEVRILMTNG |
| | | VDVNAQDQNGSTPLHLAAWDGHLEI |
| | | VEVLLKYGADVNARDLLGQTPLHLA |
| | | AINGHLEIVEVLLKHGADV |
| | | NASDRYGLTPLHLATWIGHLEIVEV |
| | | LLKHGADVNAQDKFGKTPFDLAIDN |
| | | GNEDIAEVLQKAA |
| 22 | 53F1 | DLGKKLLEAARAGQDDEVRILMANG |
| | | ADVNAEDTQGNTPLHLAAWHGHLEI |
| | | VEVLLKYGADVNASDIIGQTPLHLA |
| | | ALNGHLEIVEVLLKNGADV |
| | | NAEDRWGVTPLHLAAWDGHLEIVEV |
| | | LLKHGADVNAQDKFGKTPFDLAIDN |
| | | GNEDIAEVLQKAA |
| 23 | 53F4 | DLGKKLLEATRAGQDDEVRILMTNG |
| | | ADVNALDQAGSTPLHLAAWSGHLEI |
| | | VEVLLKYGTDVNARDVKGNTPLHLA |
| | | ANVGHLEIVEVLLKYGADV |
| | | NATDNWGHTPLHLAAFWDHLEIVEV |
| | | LLKYGADVNAQDKFGKTPFDLAIDN |
| | | GNEDIAEVLQKAA |
| 24 | 53F5 | DLGKKLLEAARAGQDDEVRILMANG |
| | | ADVNASDSVGNTPLHLAAWHGHLEI |
| | | VEVLLKYSADVNASDVSGQTPLHLA |
| | | ALQGHLEIVEVLLKCGADV |
| | | NAHDRWGLTPLHLAAHQGHLEIVEV |
| | | LLKYGADVNAQDKFGKTPFDLAIDN |
| | | GNEDIAEVLQKAA |
| 25 | 53F6 | DLGKKLLEASRAGQDDEVRILMANG |
| | | ADVNAQDRYGTTPLHLAAWHGHLEI |
| | | VEVLLKHGADVNANDVKGNTPLHLA |
| | | ANVGHLEIVEVLLKYGAD |
| | | VNAADNWGHTPLHLAAFWGHLEIVE |
| | | VLLKYGADVNAQDKFGKTPFDLAID |
| | | NGNEDIAEVLQKAA |
| 26 | 53G1 | DLGKKLLEAARAGQDDEVRILIANG |
| | | ADVNASDSVGNTPLHLAAWHGHLEI |
| | | VEVLLKYGADVNASDVSGQTPLHLA |
| | | ALQGHLEIVEVLLKYGADV |
| | | NAHDRWGLTPLHLAAHQGHLEIVEV |
| | | LLKYGADVNAQDKFGKTPFDLAIDN |
| | | GNEDIAEVLQKAA |
| 27 | 53G3 | DLGKKLLEVARAGQDDEVRILMANG |
| | | ADVNARDDAGSTPLHLAAWHGHLEI |
| | | VEVLLKYGADVNAKDIAGYTPLHLA |
| | | AVQGHLEIVEVLLKYGADV |
| | | NAKDRHGVTPLHLAAFQSHLEIVEV |
| | | LLKHGADVNAQDKFGKTPFDLAIDN |
| | | GNEDIAEVLQKAA |
| 28 | 63C5 | DLGKKLLEAARAGQDDEVRILMANG |
| | | ADVNAEDTQGNTPLHLAAWHGHLEI |
| | | VEVLLKYGADVNASDIIGQTPLHLA |
| | | ALNGHLEIVEVLLKYGADVN |
| | | AVDRYGDTPLHLAAWDGHLEIVEVL |
| | | LKHGADVNAQDKFGKTPFDLAIDNG |
| | | NEDIAEVLQKAA |

### Example 3: Generation of Tagged DARPins for Functionalization of Nanoparticles

63H6 DARPin with an N-terminal H6 and HA tag (for purification and detection) and a C-terminal Cysteine or Polyglutamate tag (E10, E20) have been generated for conjugation of LNPs and Polyplexes:

>H6-HA-63H6-Cys
MRGSHHHHHHGSYPYDVPDYAAAQPADLGKKLLEAAR
AGQDDEVRILMANGADVNASDSVGNTPLHLAAWHGHL
EIVDVLLKYGADVNASDVSGQTPMHLAALQGHLEIVE
VLLKYGADVNTHDRWGLTPLHLAAHQGHLEIVEVLLK
HGADVNAQDKFGKTPFDLAIDNGNEDIAEVLQKAAGG
C
>H6-HA-63H6-E10
MRGSHHHHHHGSYPYDVPDYAAAQPADLGKKLLEAAR
AGQDDEVRILMANGADVNASDSVGNTPLHLAAWHGHL
EIVDVLLKYGADVNASDVSGQTPMHLAALQGHLEIVE
VLLKYGADVNTHDRWGLTPLHLAAHQGHLEIVEVLLK
HGADVNAQDKFGKTPFDLAIDNGNEDIAEVLQKAAGG
SEEEEEEEEEE
>H6-HA-63H6-E20
MRGSHHHHHHGSYPYDVPDYAAAQPADLGKKLLEAAR
AGQDDEVRILMANGADVNASDSVGNTPLHLAAWHGHL
EIVDVLLKYGADVNASDVSGQTPMHLAALQGHLEIVE
VLLKYGADVNTHDRWGLTPLHLAAHQGHLEIVEVLLK
HGADVNAQDKFGKTPFDLAIDNGNEDIAEVLQKAAGG
SEEEEEEEEEEEEEEEEEEEE

To this end, corresponding gene fusions were cloned into pET-21a expression vector. Recombinant protein production was carried out using E. coli BL21(DE3) cells carrying the pET-21a vector encoding for the respective tagged DARPin variant in a 1 L scale at 37° C., 120 rpm. After the culture reached an OD₆₀₀ of approximately 0.5-0.7, protein expression was induced by adding 1 mL 1 M IPTG and incubation at 37° C., 120 rpm for additional 3 h. Afterwards, E. coli cells were harvested via centrifugation, re-suspended in 25 mL IMAC equilibration buffer and lysed by 5 consecutive sonification cycles. After centrifugation of cell debris (15.000×g for 30 min, 4° C.), the supernatant was purified by IMAC with a 1 mL HisTrap column using an ÅKTAprime™ plus system and a linear gradient from 10-500 mM imidazole in 20 min. The DARPin protein containing fractions were collected and dialyzed against PBS or 25 mM HEPES, pH 7.5, 10% Trehalose. Purity of all tagged DARPin proteins was >90% as judged by SDS-PAGE analysis (FIG. 6). In the non-reduced H6-HA-63H6-Cys sample an additional band is visible that corresponds to S—S bridged dimeric species. In the functionalization process, these dimers are removed via TCEP incubation.

To assess retained functional properties and binding to CD8 of tagged 63H6 DARPin variants, cellular binding analysis using human PBMC was performed. 3×10⁶ human PBMC were washed once with wash buffer (DPBS, 5% FCS, 5 mM EDTA), harvested by centrifugation at 300×g for 5 min and incubated with 2 μM or 1 μM DARPin in 100 μL for 1 h at 4° C. Afterwards, cells were washed twice with wash buffer, centrifuged (300×g for 5 min) and stained in 100 μL fluorescently labelled antibody mixture (anti-CD4-BV421, anti-CD3-FITC, anti-his-APC) for 1 h at 4° C. Cells were washed twice with wash buffer and once with DPBS. Staining of dead cells was performed in 100 μL fixable dye eFluor 780™ diluted 1:750 in DPBS for 20 min at 4° C. Subsequently, cells were washed twice with wash buffer and re-suspended in 100 μL wash buffer for flow cytometry analysis. Measurement was performed on BD FACSCanto II and data was analyzed using FlowJo X. Specific binding of H6-HA-63H6-Cys, H6-HA-63H6-E10 and H6-HA-63H6-E20 towards human CD8⁺ T-cell population was demonstrated on three independent PBMC donors (FIG. 7).

### Example 4: CD8-Specific Transfection by LNPs Functionalized with DARPins

LNPs consist of different lipids that are capable of self-assembly into NPs of approx. 50-150 nm in diameter. Cargo as RNA or DNA for gene delivery purpose can be encapsulated into these NPs by mixing with the lipid mixture during the self-assembly process. The so called PEG-lipid has a stabilizing function and is exposed at the outside of the LNP. Therefore, it is an optimal candidate for attachment of targeting ligands like DARPins to gain functionalization of LNPs. To achieve attachment, click chemistry displays a promising approach. The Cystein/Maleimide reaction is well-known from antibody-drug conjugation already applied in clinics. Therefore, CD8-DARPin constructs with terminal Cystein (CD8-DARPinSH) were produced in E. coli and also a PEG-Lipid with a terminal Maleimide group was synthesized. Next, LNPs with exposed Maleimide were generated (LNP-Mal) and functionalized in a second step by addition of CD8-specific DARPinSH (clones 63H6 and 63A4). Gel electrophoresis of free DARPin, LNP-Mal alone as well as DARPin plus LNP-Mal or LNP with exposed Azide (LNP-N3) as control were performed (FIG. 8A). While free DARPin could only be detected in case of incompatible reactive groups (LNP-N3), it can be assumed that all CD8-DARPins were attached to Maleimide groups on LNPs. The functionalization with DARPins slightly increases the diameter of the LNPs without any effect to the coherence of the LNP or size distribution measured by the polydispersity index (FIG. 8B). Subsequently, those DARPin-functionalized LNPs were tested on CD8⁺ and CD8⁻ Jurkat cell lines for delivery of Luciferase-mRNA (FIG. 8C, D). Indeed, only in CD8⁺ Jurkat cells a 10-100× higher signal could be detected with CD8-DARPin-functionalized LNPs in comparison to non-functionalized LNPs or LNP-N3 control. Functionalization of LNPs with CD8-DARPinSH showed likewise an improvement in transfection of primary human T cells (FIG. 8E).

### Example 5: CD8-Specific Transfection by PLX Functionalized with DARPins

PLX consist of a cationic core polymer that encapsulates the nucleic acid cargo. This core complex is stable and has transfection ability comparable to LNPs. By adding an anionic polymer to the core complex shielding the positive charge the transfection potential can be reduced. Functionalization of PLX with targeting ligands can restore the transfection potential and improve its specificity. Attachment of targeting ligands like CD8-DARPins to PLX can be achieved by electrostatic attraction between the cationic core polymer and an anionic polymer linked to the targeting ligand. It was previously described that coupling of the targeting ligand to poly-glutamic-acid (PGA) via reactive ester chemistry displays a feasible approach (Smith et al., 2017). To avoid any additional chemical modifications of the ligand, we produced CD8-specific DARPin (clone 63H6) with a tag consisting of 20 glutamic acid repetitions (CD8-DARPinE20). PLX were incubated with different amounts of CD8-DARPinE20 (indicated as w/w ratio) to follow their change in physicochemical characteristics in dependence of CD8-DARPinE20 decoration. Gel electrophoresis revealed that free DARPinE20 is not detectable in presence of the core PLX at any of the tested w/w ratios demonstrating that all E20-tagged DARPin is bound to the core particle (FIG. 9A). DLS measurements revealed that the decoration of the core PLX with CD8-DARPinE20 goes along with a significant size increase of the particles which is more pronounced at higher w/w ratios (FIG. 9B). Furthermore, a concomitant drop of particle surface charge (expressed as zeta potential) could be observed (FIG. 9C). These results are in line with expectations as absorption of CD8-DARPinE20 on the particle surface should lead to size increase and screening of the core particle's positive charge. Subsequently, also DARPin-decorated PLX were tested on human T cells but now for delivery of Luciferase- and Thy1.1-mRNA encapsulated in the same PLX (mixed in a 50/50 ratio). Here not only the Luciferase signal but also surface expression of the murine marker Thy1.1 detected via flow cytometry allowed determination of successful T-cell transfection. CD8-DARPinE20-decorated PLXs showed enhanced delivery of both RNA cargos to CD8⁺ but not to CD8⁻ Jurkat cells (FIG. 9D, E). Importantly, for all control PLXs (non-functionalized, irrelevant DARPin decoration, without CD8-DARPin attachment) no increased transfection was observed. Functionalization of PLXs with CD8-DARPinE20 showed likewise an improvement in transfection of primary human T cells without effecting viability (FIG. 9F). Flow cytometric analysis of transfection also enabled discrimination between CD4⁺ and CD8⁺ T cells and further indicated that CD8-DARPinE20-decorated PLX specifically transfect only CD8+ T cells (FIG. 9G).

### Example 6: CD8-Specific Transfection of DARPin-Decorated LNPs In Vivo

As a next step, we assessed the potential of LNPs functionalized by Cystein/Maleimide reaction to target human T cells in viva Therefore, immunodeficient mice were transplanted with human PBMC and after 21 days treated with 20 μg mRNA (Luciferase and Thy1.1, 50/50) encapsulated either in non-functionalized or CD8-DARPin-functionalized LNPs. One day after LNP administration, Luciferase signal was detected via bioluminescence imaging in situ, showing that LNPs acquired transfection of spleen resident cells next to the targeting of hepatocytes due to functionalization (FIG. 10A). These data indicate that LNPs can be retargeted to transfect a secondary lymphatic tissue where T cells are located. Analysis of Thy1.1 expression by flow cytometric analysis of peripheral blood further revealed that human (CD45⁺) CD8⁺ but not CD4⁺ T cells were transfected (FIG. 10B).

### Example 7: CD8-Specific Transfection of DARPin-Decorated PLX In Vivo

Furthermore, we assessed the potential of CD8-DARPinE20-decorated PLXs to target human T cells in vivo. Therefore, immunodeficient mice were transplanted with human PBMC and after 21 days treated with 20 μg mRNA (Luciferase and Thy1.1, 50/50) encapsulated either in non-functionalized or CD8-DARPinE20-decorated PLXs. One day after PLX administration, Luciferase signal was detected via bioluminescence imaging in situ, showing that PLX acquired transfection of spleen resident cells due to functionalization. This indicates that also PLXs can be retargeted to transfect a secondary lymphatic tissue where T cells are located. Analysis of Thy1.1 expression by flow cytometric analysis of peripheral blood further revealed that human (CD45+) CD8⁺ but not CD4⁺ T cells were transfected.

### Example 8: Functionalized Nanoparticles as Vehicles for Delivery of Mixed RNA/DNA Cargo

As mentioned above, in vivo genome engineering requires delivery of gene editing tools. However, such tools to date rely on a DNA template while enzymes can be encoded as mRNA. We could clearly show that our NPs decorated with CD8-DARPins by our in-house developed functionalization strategies are able to target human CD8⁺ T cells in vitro and in vivo. To enable genome engineering we tested if mixed cargo of DNA and RNA is sufficiently encapsulated and delivered to T cells. Therefore, we isolated as before CD8⁺ T cells from peripheral blood of a healthy donor and treated 1×10⁶ target cells with 50 ng of mixed cargo (minicircle DNA encoding Venus, an improved version of the Yellow Fluorescent Protein derived from Aequorea Victoria, and Thy1.1-mRNA, 50/50) encapsulated either in non-functionalized or CD8-DARPinE20-decorated PLX. One day after PLX administration RNA expression was detected by flow cytometry (FIG. 11). Following CD3/CD28 bead stimulation, proliferating human T cells showed strong expression of the gene of interest encoded on the minicircle DNA. These findings indicate that a single CD8-DARPin-functionalized NP batch is able to delivery DNA and RNA at the same time.

1. A method for preparing immune effector cells genetically modified to express an antigen receptor, comprising contacting the immune effector cells with particles comprising a nucleic acid encoding the antigen receptor and a targeting molecule for targeting the immune effector cells, wherein the targeting molecule is an ankyrin repeat protein.

2. The method of claim 1, wherein contacting the immune effector cells with the particles delivers the nucleic acid to the immune effector cells.

3. The method of claim 1 or 2, wherein the immune effector cells to be genetically modified are present in vivo or in vitro.

4. The method of any one of claims 1 to 3, wherein the immune effector cells to be genetically modified are present in vivo.

5. The method of anyone of claims 1 to 4, wherein the immune effector cells to be genetically modified are present in vivo in a subject and the method comprises administering the particles to the subject.

6. A method for treating a subject comprising:
(i) preparing in vitro immune effector cells genetically modified to express an antigen receptor using a method comprising contacting the immune effector cells with particles comprising a nucleic acid encoding the antigen receptor and a targeting molecule for targeting the immune effector cells, wherein the targeting molecule is an ankyrin repeat protein, and
(ii) administering the immune effector cells genetically modified to express an antigen receptor to the subject.

7. The method of claim 6, wherein contacting the immune effector cells with the particles delivers the nucleic acid to the immune effector cells.

8. A method for treating a subject comprising:
administering to the subject particles comprising a nucleic acid encoding an antigen receptor and a targeting molecule for targeting immune effector cells, wherein the targeting molecule is an ankyrin repeat protein.

9. The method of claim 8, wherein the particles deliver the nucleic acid to immune effector cells in the subject.

10. The method of claim 8, wherein delivering the nucleic acid to immune effector cells generates immune effector cells genetically modified to express an antigen receptor in the subject.

11. The method of any one of claims 5 to 9, which is a method of inducing an immune response in the subject.

12. The method of claim 11, wherein the immune response is a T cell-mediated immune response.

13. The method of claim 11 or 12, wherein the immune response is an immune response to a target cell population or target tissue expressing an antigen.

14. The method of claim 13, wherein the target cell population or target tissue is cancer cells or cancer tissue.

15. The method of claim 14, wherein the cancer cells or cancer tissue is solid cancer.

16. A method for treating a subject having a disease, disorder or condition associated with expression or elevated expression of an antigen comprising:
(i) preparing in vitro immune effector cells genetically modified to express an antigen receptor targeting the antigen associated with the disease, disorder or condition or cells expressing the antigen associated with the disease, disorder or condition using a method comprising contacting the immune effector cells with particles comprising a nucleic acid encoding the antigen receptor and a targeting molecule for targeting the immune effector cells, wherein the targeting molecule is an ankyrin repeat protein, and
(ii) administering the immune effector cells genetically modified to express an antigen receptor to the subject.

17. The method of claim 16, wherein contacting the immune effector cells with the particles delivers the nucleic acid to the immune effector cells.

18. A method for treating a subject having a disease, disorder or condition associated with expression or elevated expression of an antigen comprising:
administering to the subject particles comprising a nucleic acid encoding an antigen receptor targeting the antigen associated with the disease, disorder or condition or cells expressing the antigen associated with the disease, disorder or condition and a targeting molecule for targeting immune effector cells, wherein the targeting molecule is an ankyrin repeat protein.

19. The method of claim 18, wherein the particles deliver the nucleic acid to immune effector cells in the subject.

20. The method of claim 19, wherein delivering the nucleic acid to immune effector cells generates immune effector cells genetically modified to express an antigen receptor in the subject.

21. The method of any one of claims 16 to 20, wherein the disease, disorder or condition is cancer and the antigen associated with the disease, disorder or condition is a tumor antigen.

22. The method of any one of claims 16 to 21, wherein the disease, disorder or condition is solid cancer.

23. The method of any one of claims 6 to 22, which is a method for treating or preventing cancer in a subject.

24. The method of claim 23, wherein the cancer is solid cancer.

25. The method of claim 23 or 24, wherein the cancer is associated with expression or elevated expression of a tumor antigen targeted by the antigen receptor.

26. The method of any one of claims 5 to 25, further comprising administering to the subject an antigen targeted by the antigen receptor, a polynucleotide encoding the antigen, or a host cell genetically modified to express the antigen.

27. The method of claim 26, wherein the polynucleotide is RNA.

28. The method of claim 26, wherein the host cell comprises a polynucleotide encoding the antigen.

29. The method of any one of claims 1 to 28, wherein the antigen receptor is a chimeric antigen receptor (CAR) or T cell receptor (TCR).

30. The method of any one of claims 1 to 29, wherein the nucleic acid is RNA.

31. The method of any one of claims 1 to 29, wherein the nucleic acid is DNA.

32. The method of any one of claims 1 to 7, 10 to 17, and 20 to 31, wherein the genetic modification is transient or stable.

33. The method of any one of claims 1 to 7, 10 to 17, and 20 to 32, wherein the genetic modification takes place by a virus-based method, transposon-based method, or a gene editing-based method.

34. The method of claim 33, wherein the gene editing-based method involves CRISPR-based gene editing.

35. The method of any one of claims 1 to 34, wherein the particles are non-viral particles.

36. The method of any one of claims 1 to 35, wherein the particles are lipid-based and/or polymer-based particles.

37. The method of any one of claims 1 to 36, wherein the particles are nanoparticles.

38. The method of any one of claims 1 to 37, wherein the particles are functionalized with the targeting molecule on their surface.

39. The method of any one of claims 1 to 38, wherein the particles are functionalized with the targeting molecule by linking the targeting molecule to at least one particle-forming component.

40. The method of any one of claims 1 to 39, wherein the immune effector cells are T cells.

41. The method of any one of claims 1 to 40, wherein the immune effector cells are CD8+ T cells.

42. The method of any one of claims 1 to 41, wherein the targeting molecule targets CD8.

43. The method of any one of claims 1 to 42, wherein the targeting molecule comprises a repeat module comprising the repeat consensus sequence:
N X₁ X₂ D X₃ X₄ X₅ X₆ T P X₇ H L X₈ X₉ X₁₀ X₁₁ X₁₂ H X₁₃ X₁₄ I V X₁₅ V L L K X₁₆ X₁₇ X₁₈ D X₁₉,
wherein
X₁ is any amino acid, preferably an amino acid selected from the group consisting of A, C, D, G, N, P, S, T, and V,
X₂ is any amino acid,
X₃ is any amino acid,
X₄ is any amino acid,
X₅ is any amino acid, preferably an amino acid selected from the group consisting of A, C, D, G, N, P, S, T, and V, more preferably G,
X₆ is any amino acid,
X₇ is any amino acid, preferably an amino acid selected from the group consisting of A, C, F, G, H, I, K, L, M, R, T, V, W, Y, more preferably L,
X₈ is any amino acid, preferably an amino acid selected from the group consisting of A, C, D, G, N, P, S, T, and V, more preferably A or V,
X₉ is any amino acid, preferably an amino acid selected from the group consisting of A, C, D, G, N, P, S, T, and V, more preferably A,
X₁₀ is any amino acid,
X₁₁ is any amino acid,
X₁₂ is any amino acid, preferably an amino acid selected from the group consisting of A, C, D, G, N, P, S, T, and V, more preferably G,
X₁₃ is any amino acid, preferably L,
X₁₄ is any amino acid, preferably an amino acid selected from the group consisting of D, E, H, K, and R, more preferably E,
X₁₅ is any amino acid, preferably D or E,
X₁₆ is any amino acid,
X₁₇ is any amino acid, preferably an amino acid selected from the group consisting of A, C, D, G, N, P, S, T, and V, more preferably selected from the group consisting of A, G, and S, more preferably G,
X₁₈ is any amino acid, preferably an amino acid selected from the group consisting of A, C, D, G, N, P, S, T, and V, more preferably A,
X₁₉ is any amino acid, preferably an amino acid selected from the group consisting of I, L, and V, more preferably V.

44. The method of any one of claims 1 to 43, wherein the targeting molecule comprises a repeat module comprising the repeat consensus sequence:
N X₁ X₂ D X₃ X₄ G X₆ T P L H L X₈ X₉ X₁₀ X₁₁ G H X₁₃ X₁₄ I V X₁₅ V L L K X₁₆ G A D V,
wherein
X₁ is any amino acid, preferably an amino acid selected from the group consisting of A, C, D, G, N, P, S, T, and V,
X₂ is any amino acid,
X₃ is any amino acid,
X₄ is any amino acid,
X₆ is any amino acid,
X₈ is A or V,
X₉ is any amino acid, preferably an amino acid selected from the group consisting of A, C, D, G, N, P, S, T, and V, more preferably A,
X₁₀ is any amino acid,
X₁₁ is any amino acid,
X₁₃ is any amino acid, preferably L,
X₁₄ is any amino acid, preferably an amino acid selected from the group consisting of D, E, H, K, and R, more preferably E,
X₁₅ is any amino acid, preferably D or E,
X₁₆ is any amino acid.

45. The method of any one of claims 1 to 43, wherein the targeting molecule comprises a repeat module comprising the repeat consensus sequence:
N X₁ X₂ D X₃ X₄ G X₆ T P L H L X₈ A X₁₀ X₁₁ G H L E I V X₁₅ V L L K X₁₆ G A D V,
wherein
X₁ is any amino acid, preferably an amino acid selected from the group consisting of A, C, D, G, N, P, S, T, and V,
X₂ is any amino acid,
X₃ is any amino acid,
X₄ is any amino acid,
X₆ is any amino acid,
X₈ is A or V,
X₁₀ is any amino acid,
X₁₁ is any amino acid,
X₁₅ is D or E,
X₁₆ is any amino acid.

46. The method of any one of claims 43 to 45, wherein the targeting molecule comprises at least 2 repeat modules each comprising the repeat consensus sequence, which may be identical or different.

47. The method of any one of claims 43 to 46, wherein the targeting molecule comprises between 2 and 20 repeat modules each comprising the repeat consensus sequence, which may be identical or different.

48. The method of any one of claims 43 to 47, wherein the targeting molecule comprises 3 repeat modules each comprising the repeat consensus sequence, which may be identical or different.

49. The method of any one of claims 1 to 48, wherein the targeting molecule comprises 3 repeat modules, wherein
the first repeat module of the targeting molecule comprises the consensus sequence
N A X₂ D X₃ X₄ G X₆ T P L H L X₈ A W H G H L E I V X₁₅ V L L K X₁₆ G A D V,
the second repeat module of the targeting molecule comprise the consensus sequence
N A X₂ D X₃ X₄ G X₆ T P L H L A A X₁₀ X₁₁ G H L E I V E V L L K X₁₆ G A D V, and
the third repeat module of the targeting molecule comprise the consensus sequence
N X₁ X₂ D X₃ X₄ G X₆ T P L H L A A X₁₀ X₁₁ G H L E I V E V L L K X₁₆ G A D V,
wherein
X₁ is any amino acid, preferably an amino acid selected from the group consisting of A, C, D, G, N, P, S, T, and V,
X₂ is any amino acid,
X₃ is any amino acid,
X₄ is any amino acid,
X₆ is any amino acid,
X₈ is A or V,
X₁₀ is any amino acid,
X₁₁ is any amino acid,
X₁₅ is D or E,
X₁₆ is any amino acid, preferably an amino acid selected from the group consisting of Y, H, and N.

50. The method of any one of claims 1 to 49, wherein the targeting molecule comprises at least one repeat module each comprising a sequence selected from the group of repeat modules 1, 2 and 3 of SEQ ID Nos: 1 to 28 as shown in FIG. 5.

51. The method of any one of claims 1 to 50, wherein the targeting molecule comprises 3 repeat modules, wherein repeat module 1 is selected from the group of repeat modules 1 of SEQ ID Nos: 1 to 28 as shown in FIG. 5, repeat module 2 is selected from the group of repeat modules 2 of SEQ ID Nos: 1 to 28 as shown in FIG. 5, and repeat module 3 is selected from the group of repeat modules 3 of SEQ ID Nos: 1 to 28 as shown in FIG. 5.

52. The method of any one of claims 1 to 51, wherein the targeting molecule comprises 3 repeat modules, wherein repeat module 1, repeat module 2, and repeat module 3 are the repeat module 1, repeat module 2, and repeat module 3 of a sequence selected from the group consisting of SEQ ID Nos: 1 to 28 as shown in FIG. 5.

53. The method of any one of claims 43 to 52, wherein the repeat modules are present in a repeat domain.

54. The method of claim 53, wherein the repeat domain further comprises an N- and/or a C-terminal capping module.

55. The method of any one of claims 1 to 54, wherein the targeting molecule comprises a sequence selected from the group consisting of SEQ ID Nos: 1 to 28.

56. A molecule comprising an ankyrin repeat protein targeting immune effector cells.

57. The molecule of claim 56, wherein the immune effector cells are T cells.

58. The molecule of claim 56 or 57, wherein the immune effector cells are CD8+ T cells.

59. The molecule of any one of claims 56 to 58, wherein the molecule targets CD8.

60. The molecule of any one of claims 56 to 59, wherein the ankyrin repeat protein comprises a repeat module comprising the repeat consensus sequence:
N X₁ X₂ D X₃ X₄ X₅ X₆ T P X₇ H L X₈ X₉ X₁₀ X₁₁ X₁₂ H X₁₃ X₁₄ I V X₁₅ V L L K X₁₆ X₁₇ X₁₈ D X₁₉,
wherein
X₁ is any amino acid, preferably an amino acid selected from the group consisting of A, C, D, G, N, P, S, T, and V,
X₂ is any amino acid,
X₃ is any amino acid,
X₄ is any amino acid,
X₅ is any amino acid, preferably an amino acid selected from the group consisting of A, C, D, G, N, P, S, T, and V, more preferably G,
X₆ is any amino acid,
X₇ is any amino acid, preferably an amino acid selected from the group consisting of A, C, F, G, H, I, K, L, M, R, T, V, W, Y, more preferably L,
X₈ is any amino acid, preferably an amino acid selected from the group consisting of A, C, D, G, N, P, S, T, and V, more preferably A or V,
X₉ is any amino acid, preferably an amino acid selected from the group consisting of A, C, D, G, N, P, S, T, and V, more preferably A,
X₁₀ is any amino acid,
X₁₁ is any amino acid,
X₁₂ is any amino acid, preferably an amino acid selected from the group consisting of A, C, D, G, N, P, S, T, and V, more preferably G,
X₁₃ is any amino acid, preferably L,
X₁₄ is any amino acid, preferably an amino acid selected from the group consisting of D, E, H, K, and R, more preferably E,
X₁₅ is any amino acid, preferably D or E,
X₁₆ is any amino acid,
X₁₇ is any amino acid, preferably an amino acid selected from the group consisting of A, C, D, G, N, P, S, T, and V, more preferably selected from the group consisting of A, G, and S, more preferably G,
X₁₈ is any amino acid, preferably an amino acid selected from the group consisting of A, C, D, G, N, P, S, T, and V, more preferably A,
X₁₉ is any amino acid, preferably an amino acid selected from the group consisting of 1, L, and V, more preferably V.

61. The molecule of any one of claims 56 to 60, wherein the ankyrin repeat protein comprises a repeat module comprising the repeat consensus sequence:
N X₁ X₂ D X₃ X₄ G X₆ T P L H L X₈ X₉ X₁₀ X₁₁ G H X₁₃ X₁₄ I V X₁₅ V L L K X₁₆ G A D V,
wherein
X₁ is any amino acid, preferably an amino acid selected from the group consisting of A, C, D, G, N, P, S, T, and V,
X₂ is any amino acid,
X₃ is any amino acid,
X₄ is any amino acid,
X₆ is any amino acid,
X₈ is A or V,
X₉ is any amino acid, preferably an amino acid selected from the group consisting of A, C, D, G, N, P, S, T, and V, more preferably A,
X₁₀ is any amino acid,
X₁₁ is any amino acid,
X₁₃ is any amino acid, preferably L,
X₁₄ is any amino acid, preferably an amino acid selected from the group consisting of D, E, H, K, and R, more preferably E,
X₁₅ is any amino acid, preferably D or E,
X₁₆ is any amino acid.

62. The molecule of any one of claims 56 to 61, wherein the ankyrin repeat protein comprises a repeat module comprising the repeat consensus sequence:
N X₁ X₂ D X₃ X₄ G X₆ T P L H L X₈ A X₁₀ X₁₁ G H L E I V X₁₅ V L L K X₁₆ G A D V,
wherein
X₁ is any amino acid, preferably an amino acid selected from the group consisting of A, C, D, G, N, P, S, T, and V,
X₂ is any amino acid,
X₃ is any amino acid,
X₄ is any amino acid,
X₆ is any amino acid,
X₈ is A or V,
X₁₀ is any amino acid,
X₁₁ is any amino acid,
X₁₅ is D or E,
X₁₆ is any amino acid.

63. The molecule of any one of claims 60 to 62, wherein the ankyrin repeat protein comprises at least 2 repeat modules each comprising the repeat consensus sequence, which may be identical or different.

64. The molecule of any one of claims 60 to 63, wherein the ankyrin repeat protein comprises between 2 and 20 repeat modules each comprising the repeat consensus sequence, which may be identical or different.

65. The molecule of any one of claims 60 to 64, wherein the ankyrin repeat protein comprises 3 repeat modules each comprising the repeat consensus sequence, which may be identical or different.

66. The molecule of any one of claims 56 to 65, wherein the ankyrin repeat protein comprises 3 repeat modules, wherein
the first repeat module of the targeting molecule comprises the consensus sequence
N A X₂ D X₃ X₄ G X₆ T P L H L X₈ A W H G H L E I V X₁₅ V L L K X₁₆ G A D V,
the second repeat module of the targeting molecule comprise the consensus sequence
N A X₂ D X₃ X₄ G X₆ T P L H L A A X₁₀ X₁₁ G H L E I V E V L L K X₁₆ G A D V, and
the third repeat module of the targeting molecule comprise the consensus sequence
N X₁ X₂ D X₃ X₄ G X₆ T P L H L A A X₁₀ X₁₁ G H L E I V E V L L K X₁₆ G A D V,
wherein
X₁ is any amino acid, preferably an amino acid selected from the group consisting of A, C, D, G, N, P, S, T, and V,
X₂ is any amino acid,
X₃ is any amino acid,
X₄ is any amino acid,
X₆ is any amino acid,
X₈ is A or V,
X₁₀ is any amino acid,
X₁₁ is any amino acid,
X₁₅ is D or E,
X₁₆ is any amino acid, preferably an amino acid selected from the group consisting of Y, H, and N.

67. The molecule of any one of claims 56 to 66, wherein the ankyrin repeat protein comprises at least one repeat module each comprising a sequence selected from the group of repeat modules 1, 2 and 3 of SEQ ID Nos: 1 to 28 as shown in FIG. 5.

68. The molecule of any one of claims 56 to 67, wherein the ankyrin repeat protein comprises 3 repeat modules, wherein repeat module 1 is selected from the group of repeat modules 1 of SEQ ID Nos: 1 to 28 as shown in FIG. 5, repeat module 2 is selected from the group of repeat modules 2 of SEQ ID Nos: 1 to 28 as shown in FIG. 5, and repeat module 3 is selected from the group of repeat modules 3 of SEQ ID Nos: 1 to 28 as shown in FIG. 5.

69. The molecule of any one of claims 56 to 68, wherein the ankyrin repeat protein comprises 3 repeat modules, wherein repeat module 1, repeat module 2, and repeat module 3 are the repeat module 1, repeat module 2, and repeat module 3 of a sequence selected from the group consisting of SEQ ID Nos: 1 to 28 as shown in FIG. 5.

70. The molecule of any one of claims 60 to 69, wherein the repeat modules are present in a repeat domain.

71. The molecule of claim 70, wherein the repeat domain further comprises an N- and/or a C-terminal capping module.

72. The molecule of any one of claims 56 to 71, wherein the ankyrin repeat protein comprises a sequence selected from the group consisting of SEQ ID Nos: 1 to 28.

73. The molecule of any one of claims 56 to 72, which further comprises another peptide or protein component, optionally in fusion with the ankyrin repeat protein.

74. The molecule of any one of claims 56 to 73, which is a polypeptide compound.

75. The molecule of any one of claims 56 to 73, which further comprises a lipid or lipid-like component or another non-peptide component.

76. A nucleic acid encoding the molecule of any one of claims 56 to 74.

77. A host cell comprising the nucleic acid of claim 76, which optionally expresses the molecule.

78. A particle comprising the molecule of any one of claims 56 to 75.

79. The particle of claim 78, further comprising a nucleic acid encoding an antigen receptor.

80. The particle of claim 79, wherein the antigen receptor is a chimeric antigen receptor (CAR) or T cell receptor (TCR).

81. The particle of claim 79 or 80, wherein the antigen is associated with a disease, disorder or condition.

82. The particle of any one of claims 79 to 81, wherein the antigen is a tumor-associated antigen.

83. The particle of any one of claims 79 to 82, wherein the nucleic acid is RNA.

84. The particle of any one of claims 79 to 82, wherein the nucleic acid is DNA.

85. The particle of any one of claims 78 to 84, which is a non-viral particle.

86. The particle of any one of claims 78 to 85, which is a lipid-based and/or polymer-based particle.

87. The particle of any one of claims 78 to 86, which is a nanoparticle.

88. The particle of any one of claims 78 to 87, wherein the particle is functionalized with the ankyrin repeat protein on its surface.

89. The particle of any one of claims 78 to 88, wherein the particle is functionalized with the ankyrin repeat protein by linking the ankyrin repeat protein to at least one particle-forming component.

90. A composition comprising the molecule of any one of claims 56 to 75, the particle of any one of claims 78 to 89, or a plurality thereof.

91. A pharmaceutical composition comprising the molecule of any one of claims 56 to 75, the particle of any one of claims 78 to 89, or a plurality thereof.

92. A kit comprising the molecule of any one of claims 56 to 75, the nucleic acid of claim 76, the host cell of claim 77, the particle of any one of claims 78 to 89, the composition of claim 90, or the pharmaceutical composition of claim 91.

93. The kit of claim 92, further comprising instructions for using the kit in the method of any one of claims 1 to 55.

94. The particle of any one of claims 78 to 89, or a plurality thereof for use in the method of any one of claims 1 to 55.
